(19) United States
(12) Patent Application Publication
(10) Pub. No.: US 2009/0202431 A1
(43) Pub. Date: Aug. 13, 2009

(54) STEM-LIKE CELLS IN BONE SARCOMAS

(75) Inventors: Charles Parker Gibbs, JR., Gainesville, FL (US); Dennis Steindler, Gainesville, FL (US)

Correspondence Address: AKERMAN SENTERFITT, P.O. BOX 3188, WEST PALM BEACH, FL, 33402-3188, US

(73) Assignee: University of Florida Research Foundation, Inc., Gainesville, FL (US)

(21) Appl. No.: 11/910,934

(22) PCT Filed: Apr. 7, 2006
(86) PCT No.: PCT/US2006/013092

§ 371 Date: Jun. 20, 2008

Related U.S. Application Data
(60) Provisional application No. 60/669,747, filed on Apr. 8, 2005.

(51) Int. Cl. A61K 51/00; C12N 5/06; A61K 39/395; A61K 38/20; A61K 38/21; A61K 39/00; C12Q 1/02; C12Q 1/68; G01N 33/574

(52) U.S. Cl. 424/1.49; 435/325; 435/378; 424/174.1; 424/178.1; 424/85.2; 424/85.4; 424/277.1; 435/29; 435/6; 435/7.23

(57) ABSTRACT

Isolation and purification of stem cells from within a bulk sarcoma tumor. These cells express the marker genes of pluripotent embryonic stem cells, Stat 3, Oct 3/4, and Nanog. A subset of these cells show the surface marker of mesenchymal stem cells Stro-1, as well as express attributes of mesodermal, ectodermal, and endodermal differentiation. The isolation, purification and characterization of these stem cells now provides the ideal target for the development of highly effective therapies against tumors.

### CROSS REFERENCE TO RELATED APPLICATIONS

The present application claims the priority of U.S. provisional patent application No. 60/669,747, entitled “STEM-LIKE CELLS IN BONE SARCOMAS,” filed Apr. 8, 2005. The foregoing is incorporated herein by reference in its entirety.

### FIELD OF THE INVENTION

The invention relates to the isolation and characterization of stem cells. The stem cells are characterized by markers and are an important therapeutic target in cancer therapy.

### BACKGROUND

Bone sarcomas, including osteosarcoma and chondrosarcoma, are part of a group of mesenchymal malignancies that exhibit clinical, histologic and molecular heterogeneity. Osteosarcoma is the most common primary bone malignancy of childhood and adolescence. Despite advances in surgery and multi-agent chemotherapy, long term survival rates have stagnated at approximately 65%. The treatment of osteosarcoma most commonly consists of cytotoxic neoadjuvant chemotherapy followed by surgical resection, reconstruction and adjuvant chemotherapy. The duration of therapy is almost one year. The multiple drugs used in these regimens are highly toxic and carry significant morbidity and occasionally, mortality. Survival rates using this regimen have not improved over the last twenty years and is 50-60% for all corners. Survival rates of those with respectable oligometastases approaches 25% and those patients who have either bone metastases or unresectable lung metastases is close to zero. The benefit of systemic chemotherapy in the metastatic patient population is not well understood.

Chondrosarcoma, on the other hand is a disease of adults in which chemotherapy has been shown to play no role. Both tumors metastasize to the lung and unless the lung metastases can be completely resected, almost all patients with metastatic tumor succumb to their disease. Unfortunately many chondrosarcomas are large and occur in the pelvis requiring mutilative surgery to obtain adequate margins. Dedifferentiated chondrosarcoma (the highest grade) carries a two year survival rate of less than 10% regardless of treatment.

There is thus, an urgent need for better therapy for both of these mesenchymal malignancies and cancer in general.

### SUMMARY

The invention provides for the identification and isolation of a small subpopulation of self-renewing bone sarcoma cells. In particular, these cells are shown to be capable of forming suspended spherical clonal colonies, or “sarcospheres” in anchorage independent serum starved conditions. These bone sarcoma cells express the marker genes of pluripotent embryonic stem cells, Stat 3, Oct 3/4, and Nanog and this expression is preferentially expressed in sarcospheres. A subset of bone sarcoma cells show the surface marker of mesenchymal stem cells Stro-1, as well as express attributes of mesodermal, ectodermal, and endodermal differentiation.

In a preferred embodiment, the invention provides an isolated or purified population of pluripotent mammalian stem cells comprising at least one embryonic stem cell marker on the surface of the stem cells, wherein the embryonic stem cell marker is Oct 3/4 or Nanog; and, at least one stem cell marker comprising, activated Stat-3 CXCR4, CD 133, SCA-1, Tra-1-60, CD 44, CD 73, CD 90, CD 105 and Stro-1; wherein, the purified population of mammalian pluripotent stem cells, is substantially free of cells that do not express the stem cell markers. Other preferred markers include, but not limited to: tenascins, proteoglycans, glycoproteins, glycolipids and other glycoconjugates that make up morphogenetic molecules and extracellular matrix molecules and their receptors, undulins and the like.

In another preferred embodiment, the isolated stem cells are identified by markers comprising Oct 3/4, Nanog, and activated Stat-3.

In another preferred embodiment, the isolated or purified population of mammalian pluripotent stem cells are isolated from sarcomas. Preferably, the sarcoma is osteosarcoma or chondrosarcoma.

In another preferred embodiment, the isolated or purified population of mammalian pluripotent stem cells are Oct 3/4⁺.

In another preferred embodiment, the isolated or purified population of mammalian pluripotent stem cells are activated Stat-3⁺.

In another preferred embodiment, the isolated or purified population of mammalian pluripotent stem cells are Nanog⁺.

In another preferred embodiment, the isolated or purified population of mammalian pluripotent stem cells are Lin⁻.

In one preferred embodiment, a method of isolating stem cells from sarcomas comprises: isolating a sarcoma; dissociating cells from the sarcoma; culturing the cells in anchorage independent serum free media; and culturing the isolated cells in media with hormones and growth factors. Preferably, the hormone is progesterone and the growth factors comprise epidermal growth factor (EGF) and fibroblast growth factor.

In a preferred embodiment, the stem cells are identified by markers comprising: Oct 3/4, Nanog, activated Stat-3, CXCR4, CD 133, SCA-1, Tra-1-60, CD 44, CD 73, CD 90, CD 105 and Stro-1.

In a preferred embodiment, a method of treating tumors comprises targeting stem cells expressing at least one marker selected from: Oct 3/4, Nanog, activated Stat-3, CXCR4, CD 133, SCA-1, Tra-1-60, CD 44, CD 73, CD 90, CD 105 and Stro-1; inhibiting proliferation and/or lysing the stem cells; and, inhibiting stem cell migration to a tumor and/or stem cell differentiation in a tumor; wherein, growth and/or metastasis of a tumor is inhibited. Preferably, the stem cells are targeted with antibodies directed to any one marker selected from: Oct 3/4, Nanog, activated Stat-3, CXCR4, CD 133, SCA-1, Tra-1-60, CD 44, CD 73, CD 90, CD 105 and Stro-1.

In one preferred embodiment, the antibodies are fused to therapeutic effector molecules. Preferably, the therapeutic effector domain is selected from the group consisting of endostatin, angioarrestin, angiostatin (plasminogen fragment), anti-angiogenic antithrombin III, cartilage-derived inhibitor (CDI), CD59 complement fragment, fibronectin fragment, gro-beta, heparinases, heparin hexasaccharide fragment, human chorionic gonadotropin (hCG), interferon alpha/beta/gamma, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-β), vasculostatin, and vasostatin (calreticulin fragment). Therapeutic molecules also include: endostatin, angiostatin, basement-membrane collagen-derived anti-angiogenic factors tumstatin, canstatin, or arrestin. The therapeutic can also comprise chemokines, radionuclides and/or interferon. Examples of nuclides are: ⁹⁰Y, ¹³¹I, ¹¹¹In, ¹²⁵I. Therapeutic molecules can be cytolytic molecules such as, TNF, enzymes, mediators of apoptosis and/or toxin. Examples of toxins are: ricin, abrin, diphtheria, gelonin, Pseudomonas exotoxin A, Crotalus durissus terrificus toxin, Crotalus adamenteus toxin, Naja naja toxin, and Naja mocambique toxin. Examples of mediators of apoptosis include ICE-family of cysteine proteases, apoptin, Bcl-2 family of proteins, Bax, bclxs and caspases.

In another preferred embodiment, a method of treating a tumor comprises isolating stem cells from a patient, whereby the stem cells are identified by Oct 3/4, Nanog, activated Stat-3, CXCR4, CD 133, SCA-1, Tra-1-60, CD 44, CD 73, CD 90, CD 105 and Stro-1; isolating antigen specific lymphocytes from the patient; co-culturing the stem cells and lymphocytes ex-vivo thereby priming the lymphocytes; isolating activated lymphocytes from the co-culture; re-infusing the activated lymphocytes into the patient; thereby, activating an immune response to said stem cells and preventing self-renewal of the tumor.

Preferably, the antigen-specific lymphocytes are T- or B-lymphocytes. The T lymphocytes are identified as CD4⁺ and/or CD8⁺.

In another preferred embodiment, a method of treating a tumor comprises isolating stem cells from a patient, whereby the stem cells are identified by Oct 3/4, Nanog, activated Stat-3, CXCR4, CD 133, SCA-1, Tra-1-60, CD 44, CD 73, CD 90, CD 105 and Stro-1; isolating antigen presenting cells from the patient; co-culturing the stem cells and antigen presenting cells ex-vivo; isolating the antigen presenting cells from the co-culture; re-infusing the antigen presenting cells into the patient; thereby, activating an immune response to said stem cells and preventing self-renewal of the tumor. Preferably, the antigen presenting cells are dendritic cells, more preferably, the antigen presenting cell is an immature dendritic cell.

In another preferred embodiment, a method of treating cancer comprises administering at least one protein or peptide to a patient comprising Oct 3/4, Nanog, activated Stat-3, CXCR4, CD 133, SCA-1, Tra-1-60, CD 44, CD 73, CD 90, CD 105 and Stro-1; thereby stimulating an immune response against stem cells expressing said proteins.

In another preferred embodiment, a method of selectively treating cancer comprises administering to a patient in need of treatment a therapeutically effective amount of a composition comprising antigen-activated dendritic cells, wherein dendritic cells are produced from proliferating cell cultures by a method comprising providing a tissue source comprising dendritic cell precursors; culturing the tissue source on a substrate and in culture medium to expand the number of dendritic cell precursors by allowing the dendritic cell precursors to proliferate; the culture medium comprises GM-CSF and at least one other factor which inhibits the proliferation or maturation of non-dendritic cell precursors thereby increasing the proportion of dendritic cell precursors in the culture; culturing the dendritic cell precursors for a period of time sufficient to allow them to mature into mature dendritic cells; the dendritic cells are pulsed with stem cell derived antigen; the dendritic cells process the stem-cell derived antigen to produce modified antigen, which is expressed by the dendritic cells and which activate an in vivo immune response which destroys stem cells expressing the stem-cell derived antigen. Preferably, the stem-cell derived antigen comprises Oct 3/4, Nanog, activated Stat-3, CXCR4, CD 133, SCA-1, Tra-1-60, CD 44, CD 73, CD 90, CD 105 and Stro-1 and the immune cells selectively target stem cells expressing Oct 3/4, Nanog, activated Stat-3, CXCR4, CD 133, SCA-1, Tra-1-60, CD 44, CD 73, CD 90, CD 105 and Stro-1. Preferably, the immune cells are CD4+ T lymphocytes and/or CD8+ T lymphocytes.

In a preferred embodiment, candidate therapeutic compounds are directed to stem cells expressing Oct 3/4, Nanog, Stro-1. Candidate compounds can serve to prevent the stem cells from maturing into a tumor cell within the bulk tumor. Alternatively, these cells can be targeted by compounds which are cytotoxic.

In a preferred embodiment, a method of identifying candidate therapeutic compounds comprises culturing stem cells expressing at least one marker selected from: Oct 3/4, Nanog, activated Stat-3, CXCR4, CD 133, SCA-1, Tra-1-60, CD 44, CD 73, CD 90, CD 105 and Stro-1 with a candidate therapeutic agent; identifying candidate therapeutic agents which inhibit proliferation or growth, and/or lyse the stem cells and/or inhibit stem cell migration to a tumor and/or stem cell differentiation in a tumor; wherein, growth and/or metastasis of a tumor is inhibited, and identifying a candidate therapeutic agent. Preferably, a candidate therapeutic agent comprises organic molecules, inorganic molecules, vaccines, antibodies, nucleic acid molecules, proteins, peptides and vectors expressing nucleic acid molecules.

In another preferred embodiment, a method of treating cancer comprises administering antibodies to stem cell biomarkers wherein the antibodies are specific for at least one marker selected from: Oct 3/4, Nanog, activated Stat-3, CXCR4, CD 133, SCA-1, Tra-1-60, CD 44, CD 73, CD 90, CD 105 and Stro-1 and/or ligands thereof; inhibiting proliferation or growth, and/or lysing the stem cells and/or inhibiting stem cell migration to a tumor and/or stem cell differentiation in a tumor; wherein, growth and/or metastasis of a tumor is inhibited, and treating cancer.

In another preferred embodiment, a method of producing self-renewing pluripotent stem cell clones comprises culturing sarcospheres; dissociating sarcospheres into single cells; culturing said single cells to near confluence and harvesting; reseeding harvested cells into suspension cultures; identifying self-renewing cells by formation of secondary spheres; thereby producing self-renewing pluripotent stem cell clones.

Other aspects of the invention are described infra.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIGS. 1A and 1B are phase-contrast images showing the isolation of self-renewing cells from bone sarcomas using sarcospheres cultured in chemically-defined medium and having endogenously activated Stat-3 signaling. Phase-contrast images of monoclonal sarcospheres suspended in MC (FIG. 1A), show compact, undifferentiated morphology. Clones were grown for 10-14 days in N2 medium without serum and LIF, supplemented with 0.8% MC, and 10 ng/ml EGF and FGF2, in 2 cm²-wells treated with anti-adhesive material. Sarcospheres transferred from MC and attached to substratum (FIG. 1B) show flat spread cells expanding from spheres.

FIGS. 2A-2D show that genes specific to ESCs show increased expression in sphere cultures derived from bone sarcomas. FIG. 2A is a Western blot showing monolayer and sarco-sphere (SP) cultures from five osteosarcoma (OS) and three chondro-sarcomas (CS) were analyzed for Oct-4, Nanog and STAT3 mRNA by RT-PCR; β-actin expression was used as a positive control. Sphere cultures demonstrate increased transcription of both Oct-4 and Nanog over adherent cultures; STAT3 expression was uniform between both culture types. FIG. 2B is a densitometer scan showing relative band intensities for Oct-4 and Nanog for each culture from FIG. 2A were quantitated by densitometry, normalized relative to β-actin and plotted on the graph shown (Oct-4, x-axis; Nanog, y-axis). As indicated by the grouping, the sphere cultures of each sarcoma showed significantly greater expression of both Oct-4 and Nanog than adherent monolayer cultures (p<0.05, Pearson's correlation). FIG. 2C is a Western blot analysis of lysates from representative bone sarcoma cell cultures for protein expression of Oct-4, STAT3 and activated (phosphorylated, p) STAT3. β-actin was used as a positive control for loading, membrane transfer and iimmunoblotting. All cultures showed positive staining of protein bands of the appropriate sizes as indicated. FIG. 2D is a scan of a photograph showing small and large sarcospheres embedded in fibrin and then paraffin and stained using immunohistochemistry for Nanog and Oct-4 as indicated. Small spheres show intense staining in cells in the periphery. Large spheres show similar numbers of darkly staining cells with dramatically increased numbers of poorly staining cells in the interior of the sphere.

FIG. 3 is a scan of a photograph showing Oct 3/4 and Nanog nuclear stained sarcoma tissue blocks. Each block shows a representative osteosarcoma (OS-154) and chondrosarcoma (CS-187) and positive control, human fetal testis. CS-187 shows a single nuclei positive (brown) for Oct 3/4 and multiple nuclei positive (brown) for Nanog in a lung metastasis from a chondrosarcoma. OS154 sections demonstrate scattered Oct 3/4 nuclear staining and near complete nuclear Nanog staining in a primary fibular osteosarcoma. 26 week fetal testis with scattered Oct 3/4 and Nanog nuclear staining as positive control.

FIG. 4A shows RT-PCR analysis revealing the expression of endoderm associated genes (Gata-4, Gata-6, and AFP) and the neuro-ectoderm marker, β-IIII tubulin in some bone sarcoma adherent and spherical cultures. FIG. 4B shows a Western blot analysis demonstrating protein expression in sarcoma cell culture of endoderm associated alpha-feto protein and neuro-ectoderm marker β-III tubulin. FIG. 4C-4F is a photograph showing β-III tubulin in four different representative bone sarcomas by immunofluorescent staining of cell culture and immunohistochemical staining of tissue specimens.

FIG. 5A is a histochemical stain of cells showing mesenchymal lineage and differentiation. Demonstration in osteosarcoma (OS 99-1) and chondrosarcoma (CS 828) cells of a subpopulation of Stro-1 positive cells. Activation of osteogenic and adipogenic programs of differentiation in bone sarcoma cultures grown in specific differentiation media. Osteogenic lineage indicated by expression of alkaline phosphatase and adipogenic by accumulation of lipid vacuoles stained with Oil-red-O. FIG. 5B is a blot showing preservation of multiple osteo/chondral mesenchymal lineage markers (Runx2, Runx 3, alkaline phosphatase, osteocalcin, and bone-sialo protein) in both sarcospheres and adherent cultures demonstrated by RT-PCR.

FIGS. 6A and 6B are scans of photographs showing a tumor in NOD/SCID mouse receiving 3×10⁵ cells from sarcosphere grown from osteosarcoma cells. FIG. 6A shows the lateral view, and FIG. 6B shows the dorsal view.

FIGS. 7A-7B show the characterization of osteosarcoma cells for markers of ESCs and MSCs. FIG. 7A is a scan of a photograph and scatter plot showing that the osteosarcoma culture OS 99-1 was transfected with the hOct-4-EGFP plasmid construct. EGFP expression driven by the Oct-4 promoter was visualized by fluorescence microscopy (left) and flow cytometry (right). Parallel cultures of OS 99-1 were untreated (Control) cells or were transfected with phOct-4-EGFP as indicated. FIG. 7B shows the results of cultures of OS 99-1 and MG-63 cells analyzed by flow cytometry for expression of SSEA4, a surface marker of ESCs, or Stro-1 an antigen found on bone marrow stromal cells.

### DETAILED DESCRIPTION

Isolation and purification of malignant bone sarcoma cancer stem cells (BCSS) from within the bulk sarcoma tumor. In particular, the stem cells are characterized as immortalized osteo/chondrogenic stem cells and are the only cells within a bulk tumor that are capable of metastasis and propagation of the tumor. The isolation, purification and characterization of these stem cells now provides the ideal target for the development of highly effective therapies against bone sarcomas.

### DEFINITIONS

Prior to setting forth the invention, definitions of certain terms which are used in this disclosure are set forth below:

As used herein, the singular forms “a”, “an” and “the” include plural referents unless the context clearly dictates otherwise.

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to marker “X” from specific species such as rat, mouse, or human can be selected to obtain only those polyclonal antibodies that are specifically immunoreactive with marker “X” and not with other proteins, except for polymorphic variants and alleles of marker “X”. This selection may be achieved by subtracting out antibodies that cross-react with marker “X” molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

The terms “patient” or “individual” are used interchangeably herein, and is meant a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

“Treatment” is an intervention performed with the intention of preventing the development or altering the pathology or symptoms of a disorder. Accordingly, “treatment” refers to both therapeutic treatment and prophylactic or preventative measures. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy. As used herein, “ameliorated” or “treatment” refers to a symptom which is approaches a normalized value (for example a value obtained in a healthy patient or individual), e.g., is less than 50% different from a normalized value, preferably is less than about 25% different from a normalized value, more preferably, is less than 10% different from a normalized value, and still more preferably, is not significantly different from a normalized value as determined using routine statistical tests.

The “treatment of cancer or tumor cells”, refers to one or more of the following effects: (1) inhibition, to some extent, of tumor growth, including, (i) slowing down (ii) inhibiting angiogenesis and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to delay the growth of or to cause a cancer, either a sarcoma or lymphoma, or to shrink the cancer or prevent metastasis. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

The term “sarcoma” generally refers to a tumor which is made up of a substance like the embryonic connective tissue and is generally composed of closely packed cells embedded in a fibrillar or homogeneous substance. Examples of sarcomas which can be treated with the compositions described herein and optionally a potentiator and/or chemotherapeutic agent include, but not limited to a chondrosarcoma, osteosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

“T cells” or “T lymphocytes” are a subset of lymphocytes originating in the thymus and having heterodimeric receptors associated with proteins of the CD3 complex (e.g., a rearranged T cell receptor, the heterodimeric protein on the T cell surfaces responsible for antigen/MHC specificity of the cells). T cell responses may be detected by assays for their effects on other cells (e.g., target cell killing, activation of other immune cells, such as B-cells) or for the cytokines they produce.

As used herein, “allogeneic” is used to refer to immune cells derived from non-self major histocompatibility complex donors. HLA haplotypes/allotypes vary from individual to individual and it is often helpful to determine the individual's HLA type. The HLA type may be determined via standard typing procedures.

As will be recognized by those in the art, the term “host compatible” or “autologous” cells means cells that are of the same or similar haplotype as that of the subject or “host” to which the cells are administered, such that no significant immune response against these cells occurs when they are transplanted into a host.

“CD4” is a cell surface protein important for recognition by the T cell receptor of antigenic peptides bound to MHC class II molecules on the surface of an APC. Upon activation, naïve CD4⁺ T cells differentiate into one of at least two cell types, Th₁ cells and Th₂ cells, each type being characterized by the cytokines it produces. “Th₁ cells” are primarily involved in activating macrophages with respect to cellular immunity and the inflammatory response, whereas “Th₂ cells” or “helper T cells” are primarily involved in stimulating B cells to produce antibodies (humoral immunity). Effector molecules for Th₁ cells include, but are not limited to: IFN-γ, GM-CSF, TNF-α, CD40 ligand, Fas ligand, IL-3, TNF-β, and IL-2. Effector molecules for Th₂ cells include, but are not limited to, IL-4, IL-5, CD40 ligand, IL-3, GS-CSF, IL-10, TGF-β, and eotaxin. Activation of the Th₁ type cytokine response can suppress the Th₂ type cytokine response, and reciprocally, activation of the Th₂ type cytokine response can suppress the Th₁ type response.

The term “activated T cell,” as used herein, refers to a T cell that expresses antigens indicative of T-cell activation (that is, T cell activation markers). Examples of T cell activation markers include, but are not limited to, CD25, CD26, CD30, CD38, CD69, CD70, CD71, ICOS, OX-40 and 4-1BB. The expression of activation markers can be measured by techniques known to those of skill in the art, including, for example, Western blot analysis, northern blot analysis, RT-PCR, immunofluorescence assays, and fluorescence activated cell sorter (FACS) analysis.

The term “resting T cell,” as used herein, refers to a T cell that does not express T-cell activation markers. Resting T cells include, but are not limited to, T cells which are CD25⁻, CD69⁻, ICOS⁻, SLAM⁻, and 4-1BB⁻. The expression of these markers can be measured by techniques known to those of skill in the art, including, for example, Western blot analysis, northern blot analysis, RT-PCR, immunofluorescence assays, and fluorescence activated cell sorter (FACS) analysis.

The immune system contains a system of “dendritic cells” that are specialized to present antigens and initiate several T-dependent immune responses. Dendritic cells are distributed widely throughout the body in various tissues. Dendritic cells are found in nonlymphoid organs either close to body surfaces, as in the skin and airways, or in interstitial regions of organs like heart and liver. Dendritic cells, possibly under the control of the cytokine granulocyte macrophage colony-stimulating factor, (hereinafter GM-CSF), can undergo a maturation process that does not entail cell proliferation. Initially, the dendritic cells process and present antigens most likely on abundant, newly synthesized MHC class II molecules, and then strong accessory and cell-cell adhesion functions are acquired. Dendritic cells can migrate via the blood and lymph to lymphoid organs. There, presumably as the “interdigitating” cells of the T-area antigens can be presented to T cells in the recirculating pool

A “chemokine” is a small cytokine involved in the migration and activation of cells, including phagocytes and lymphocytes, and plays a role in inflammatory responses.

A “cytokine” is a protein made by a cell that affect the behavior of other cells through a “cytokine receptor” on the surface of the cells the cytokine effects. Cytokines manufactured by lymphocytes are sometimes termed “lymphokines.” Cytokines are also characterized as Type I (e.g. IL-2 and IFN-γ) and Type II (e.g. IL-4 and IL-10).

By the term “modulate,” it is meant that any of the mentioned activities, are, e.g., increased, enhanced, increased, agonized (acts as an agonist), promoted, decreased, reduced, suppressed blocked, or antagonized (acts as an agonist). Modulation can increase activity more than 1-fold, 2-fold, 3-fold, 5-fold, 10-fold, 100-fold, etc., over baseline values. Modulation can also decrease its activity below baseline values.

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

An “allele” or “variant” is an alternative form of a gene. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The terms “amino acid” or “amino acid sequence” refer to an oligopeptide, peptide, polypeptide, or protein sequence, or a fragment of any of these, and to naturally occurring or synthetic molecules. In this context, “fragments,” refer to fragments of protein which are preferably at least or 10 to about 30 or 50, 60, 70, 80 90 or 100 amino acids in length, more preferably at least 15, 20, 25, 30, 40, or 50 amino acids. Where “amino acid sequence” is recited to refer to an amino acid sequence of a naturally occurring protein molecule, “amino acid sequence” and like tenns are not meant to limit the amino acid sequence to the complete native amino acid sequence associated with the recited protein molecule.

### Stem Cell Isolation and Characterization

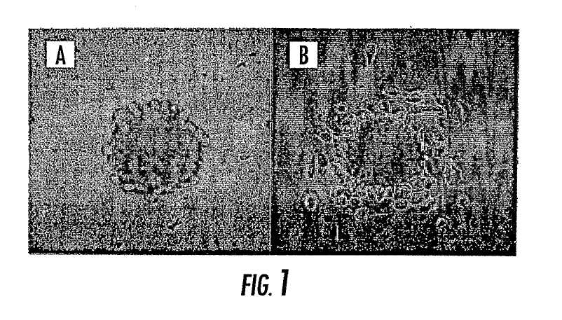

In a preferred embodiment, the stem cells form “sarcospheres” (SS) in culture. (See FIGS. 1A and 1B). These are created using an anchorage independent, serum starved system that was devised. Isolation and purification of the stem cells are described in detail in the examples which follow. The isolation was based on the hypothesis that the heterogeneity and the relative resistance to chemotherapy of tumors might be associated with the presence of tumor stem-like cells. These stem-like cells have the potential to self-renew in culture and generate a developmental hierarchy of differentiating progeny. The culture methodology of these cells was based on the idea that stressful growth conditions either de-differentiate certain cells or select for the most primitive cells in the culture by eliminating most of the differentiated cells that are unable to survive serum and anchorage withdrawal. Suspending dissociated normal and cancerous tissue in semi-solid media without serum selected for primitive clonogenic cells that are then expanded and give rise to different classes of cells, i.e. a stem/progenitor cell population of the invention, within the dissociate. Semiquantitative RT-PCR, Western analysis, and immunophenotyping were used to examine gene expression suggestive of stem cell presence. Osteogenic and adipogenic media were used to determine functional mesenchymal differentiation.

In another preferred embodiment, the stem cells are isolated based on their growing conditions. These methods are provided in U.S. Pat. No. 6,638,763 to Steindler et al., which is incorporated herein by reference, in its entirety. Briefly, cell separation and cell adhesion are manipulated using a variety of contact-limiting and contact-inhibiting factors. For example, chemical-separating agents such as mercaptoethanol, physical separating agents such as methylcellulose, and anti-adhesives such as poly 2-hydroxyethyl methacrylate are used to deter cell-cell and cell-substrate associates during the initial isolation of stem/precursor cells from the newly-dissociated brain. This allows the purification of these cells from mature, differentiated tumors that are also dissociated during the tumor dissociation procedures. The mature, differentiated tumor cells cannot survive these anti-adhesion, anti-cell interaction procedures. Thus, agents such as mercaptoethanol are always used in the first stage of isolation to help deter the survival of the more mature cellular elements (by deterring their clustering). At the same time, agents such as mercaptoethanol may have certain growth-promoting actions on the single stem/precursor cells that eventually proliferate to form these early sphere types, e.g. sarcospheres. Preferably, a method for obtaining a purified population of stem cells, comprising culturing dissociated solid tumor cells on a non-adhesive substrate in suspension culture supplemented with fetal bovine serum and methyl cellulose, where culturing under conditions that inhibit cell-cell and cell-substrate interactions results in a substantially homogeneous population of pluripotent stem cells that are free from mature, differentiated cells.

### Purification

Besides the methods used to identify and characterize the stem cells of the invention ad described in detail in the Examples which follow, the procedures can include using the identified stem cell markers which bind to the binding molecule. The binding molecule distinguishes the bound cells from unbound cells, permitting separation and isolation. For example, Oct 3/4, Nanog, Stro-1. If the bound cells do not internalize the molecule, the molecule may be separated from the cell by methods known in the art. The molecule used for isolating the purified populations of stem cells is advantageously conjugated with labels that expedite identification and separation. Examples of such labels include magnetic beads; biotin, which may be identified or separated by means of its affinity to avidin or streptavidin; fluorochromes, which may be identified or separated by means of a fluorescence-activated cell sorter (FACS, see below), and the like. Any technique may be used for isolation as long as the technique does not unduly harm the stem cells. Many such methods are known in the art.

In one embodiment, the binding molecule is attached to a solid support. Some suitable solid supports include nitrocellulose, agarose beads, polystyrene beads, hollow fiber membranes, magnetic beads, and plastic Petri dishes. For example, the binding molecule can be covalently linked to Pharmacia Sepharose 6 MB macro beads. Examples of the binding molecule include antibodies to: Oct 3/4, Nanog, activated Stat-3, CXCR4, CD 133, SCA-1, Tra-1-60, CD 44, CD 73, CD 90, CD 105 and Stro-1. Other preferred markers include, but not limited to: tenascins, proteoglycans, glycoproteins, glycolipids and other glycoconjugates that make up morphogenetic molecules and extracellular matrix molecules and their receptors, undulins and the like. For example, glycosaminoglycan (GAG)-associated molecular interactions” is intended to include the binding of a GAG to, for example, a cell surface, secreted, or extracellular protein. This term also includes any subsequent results of such protein binding such as, for example, delayed proteolytic degradation or denaturing, changes in protein conformation (which may, for example, lead to alterations of biological activity), or catalysis of a reaction between two different proteins bound to the same or different GAGs on the same or different proteoglycans. Also included is the ability of certain GAGs, e.g., heparin sulfate, to modulate the interaction of a protein to another GAG, for example, FGF-2 (basic fibroblast growth factor) to its GAG cell receptor. Other non-limiting examples include polypeptide growth factors (e.g., FGFs1-9, PDGF, HGF, VEGF, TGF-β, IL-3); extracellular matrix components (e.g., laminins, fibronectins; thrombospondins, tenascins, collagens, VonWillebrand's factor); proteases and anti-proteases (e.g., thrombin, TPA, UPA, clotting factors IX and X, PAI-1); cell-adhesion molecules (e.g., N-CAM, LI, myelin-associated glycoprotein); proteins involved in lipoprotein metabolism (e.g., APO-B, APO-E, lipoprotein lipase); cell-cell adhesion molecules (e.g., N-CAM, myelin-associated glycoprotein, selecting, pecam); angiogenin; lactoferrin; viral proteins (e.g., proteins from HIV, herpes complex) and other compounds which bind to GAG. The definition is intended to include the result of the binding of these factors to the GAG. For example, the binding of polypeptide growth factor to a GAG can result in cell proliferation, angiogenesis, inflammation, cancer, and other biologically important responses.

The exact conditions and duration of incubation for the solid phase-linked binding molecules with the crude cell mixture will depend upon several factors specific to the system employed, as is well known in the art.

Cells that are bound to the binding molecule are removed from the cell suspension by physically separating the solid support from the remaining cell suspension. For example, the unbound cells may be eluted or washed away with physiologic buffer after allowing sufficient time for the solid support to bind the stem cells. The bound cells are separated from the solid phase by any appropriate method, depending mainly upon the nature of the solid phase and the binding molecule. For example, bound cells can be eluted by enzymatically “nicking” or digesting an enzyme-sensitive “spacer” sequence between the solid phase and an antibody (e.g., antibodies directed to: Oct 3/4, Nanog, activated Stat-3, CXCR4, CD 133, SCA-1, Tra-1-60, CD 44, CD 73, CD 90, CD 105 and Stro-1). Suitable spacer sequences bound to agarose beads are commercially available from, for example, Pharmacia.

The eluted, enriched fraction of cells may then be washed with a buffer by centrifugation and preserved in a viable state at low temperatures for later use according to conventional technology. The cells may also be used immediately, for example by being infused intravenously into a recipient.

Methods for removing unwanted cells by negative selection are also known. For example, unwanted cells in a starting cell population are labeled by an antibody, or by a cocktail of antibodies, to a cell surface protein characteristic of Lin⁺ cells. The unwanted antibody-labeled cells are removed by methods known in the art. For example, the labeled cells can be immobilized on a column that binds to the antibodies and captures the cells.

Alternatively, the antibody that binds the cell surface proteins can be linked to magnetic colloids for capture of unwanted cells on a column surrounded by a magnetic field. This system is currently available through StemCell Technologies Inc., Vancouver, British Columbia, Canada. The remaining cells that flow through the column for collection are enriched in cells that do not express the cell surface proteins that the tetrameric antibodies were directed against. The antibody cocktail that can be used to deplete unwanted Lin⁺ cells can be custom made to include antibodies against lineage specific markers, such as, for example, CD2, CD3, CD4, CD5, CD8, CD10, CD11b, CD13, CD14, CD15, CD16, CD19, CD20, CD24, CD25, CD28, CD29, CD33, CD36, CD38, CD41, CD56, CD66b, CD66e, CD69, and glycophorin A. The desired cells that lack these markers are not lineage committed, i.e. Lin⁻.

In a preferred embodiment, a labeled binding molecule is bound to the stem cells, and the labeled cells are separated by a mechanical cell sorter that detects the presence of the label. The preferred mechanical cell sorter is a fluorescence activated cell sorter (FACS). FACS machines are commercially available. Generally, the following FACS protocol is suitable for this procedure: a Coulter Epics Eliter sorter is sterilized by running 70% ethanol through the systems. The lines are flushed with sterile distilled water. Cells are incubated with a primary antibody diluted in Hank's balanced salt solution supplemented with 1% bovine serum albumin (HB) for 60 minutes on ice. The cells are washed with HB and incubated with a secondary antibody labeled with fluorescein isothiocyanate (FITC) for 30 minutes on ice. The secondary label binds to the primary antibody. The sorting parameters, such as baseline fluorescence, are determined with an irrelevant primary antibody. The final cell concentration is usually set at one million cells per ml. While the cells are being labeled, a sort matrix is determined using fluorescent beads as a means of aligning the instrument. Once the appropriate parameters are determined, the cells are sorted and collected in sterile tubes containing medium supplemented with fetal bovine serum and antibiotics, usually penicillin, streptomycin and/or gentamicin. After sorting, the cells are re-analyzed on the FACS to determine the purity of the sort.

In a preferred embodiment, isolated cells from bulk tumors are enriched for stem cell populations. Preferably, cells are sorted with a FACS sorter using antibodies directed to any one of the stem cells markers. Preferred markers include, but not limited to: Oct 3/4, Nanog, activated Stat-3, CXCR4, CD 133, SCA-1, Tra-1-60, CD 44, CD 73, CD 90, CD 105 and Stro-1. As discussed supra, the cells can be enriched using either positive or negative selection procedures.

### Diagnostics, Candidate Therapeutic Compounds and Compositions

The presence of stem cells isolated from tumors indicates a role of these cells in the pathogenesis of tumors. Without wishing to be bound by theory, the stem cells within a tumor have the ability, as do normal stem cells, to self renew. These few cells divide asymmetrically, producing an identical daughter cell and a more differentiated cell that goes on to comprise the vast majority of the tumor bulk. The stem-like cell is responsible for initiating and maintaining the growth of the tumor and if not completely eradicated by surgical extirpation or chemotherapy might also be responsible for local and distant recurrence in bone sarcoma patients. Therefore, any therapy directed to these cells would be of great therapeutic value.

In a preferred embodiment, candidate therapeutic compounds are directed to stem cells expressing Oct 3/4, Nanog, Stro-1. Candidate compounds can serve to prevent the stem cells from maturing into a tumor cell within the bulk tumor. Alternatively, these cells can be targeted by compounds which are cytotoxic.

In a preferred embodiment, a method of identifying candidate therapeutic compounds comprises culturing stem cells expressing at least one marker selected from: Oct 3/4, Nanog, activated Stat-3, CXCR4, CD 133, SCA-1, Tra-1-60, CD 44, CD 73, CD 90, CD 105 and Stro-1 with a candidate therapeutic agent; identifying candidate therapeutic agents which inhibit proliferation or growth, and/or lyse the stem cells and/or inhibit stem cell migration to a tumor and/or stem cell differentiation in a tumor; wherein, growth and/or metastasis of a tumor is inhibited, and identifying a candidate therapeutic agent. Preferably, a candidate therapeutic agent comprises organic molecules, inorganic molecules, vaccines, antibodies, nucleic acid molecules, proteins, peptides and vectors expressing nucleic acid molecules. Other preferred markers include, but not limited to: tenascins, proteoglycans, glycoproteins, glycolipids and other glycoconjugates that make up morphogenetic molecules and extracellular matrix molecules and their receptors, undulins and the like. Other non-limiting examples include polypeptide growth factors (e.g., FGFs1-9, PDGF, HGF, VEGF, TGF-β, IL-3); extracellular matrix components (e.g., laminins, fibronectins; thrombospondins, tenascins, collagens, VonWillebrand's factor); proteases and anti-proteases (e.g., thrombin, TPA, UPA, clotting factors IX and X, PAI-1); cell-adhesion molecules (e.g., N-CAM, LI, myelin-associated glycoprotein); proteins involved in lipoprotein metabolism (e.g., APO-B, APO-E, lipoprotein lipase); cell-cell adhesion molecules (e.g., N-CAM, myelin-associated glycoprotein, selectins, pecam); angiogenin; lactoferrin; viral proteins (e.g., proteins from HIV, herpes complex) and other compounds which bind to GAG.

In another preferred embodiment, a method of identifying candidate therapeutic compounds comprises culturing stem cells expressing at least one marker selected from: Oct 3/4, Nanog, activated Stat-3, CXCR4, CD 133, SCA-1, Tra-1-60, CD 44, CD 73, CD 90, CD 105 and Stro-1 and identify those candidate compounds which bind to at least one of these markers. Other preferred markers include, but not limited to: tenascins, proteoglycans, glycoproteins, glycolipids and other glycoconjugates that make up morphogenetic molecules and extracellular matrix molecules and their receptors, undulins and the like. Other non-limiting examples include polypeptide growth factors (e.g., FGFs1-9, PDGF, HGF, VEGF, TGF-β, IL-3); extracellular matrix components (e.g., laminins, fibronectins; thrombospondins, tenascins, collagens, VonWillebrand's factor); proteases and anti-proteases (e.g., thrombin, TPA, UPA, clotting factors IX and X, PAI-1); cell-adhesion molecules (e.g., N-CAM, LI, myelin-associated glycoprotein); proteins involved in lipoprotein metabolism (e.g., APO-B, APO-E, lipoprotein lipase); cell-cell adhesion molecules (e.g., N-CAM, myelin-associated glycoprotein, selectins, pecam); angiogenin; lactoferrin; viral proteins (e.g., proteins from HIV, herpes complex) and other compounds which bind to GAG.

A number of suitable assay methods to detect binding of test compounds to Oct 3/4, Nanog, activated Stat-3, CXCR4, CD 133, SCA-1, Tra-1-60, CD 44, CD 73, CD 90, CD 105 and Stro-1 are known in the art, and include, but are not limited to, surface plasmon resonance (SPR)/Biacore™, fluorogenic binding assays, fluid phase binding assays, affinity chromatography, size exclusion or gel filtration, ELISA, immunoprecipitation, competitive binding assays, gel shift assays, and mass spectrometry based methods, iizter alia. Other preferred markers include, but not limited to: tenascins, proteoglycans, glycoproteins, glycolipids and other glycoconjugates that make up morphogenetic molecules and extracellular matrix molecules and their receptors, undulins and the like. Other non-limiting examples include polypeptide growth factors (e.g., FGFs1-9, PDGF, HGF, VEGF, TGF-β, IL-3); extracellular matrix components (e.g., laminins, fibronectins; thrombospondins, tenascins, collagens, VonWillebrand's factor); proteases and anti-proteases (e.g., thrombin, TPA, UPA, clotting factors IX and X, PAI-1); cell-adhesion molecules (e.g., N-CAM, LI, myelin-associated glycoprotein); proteins involved in lipoprotein metabolism (e.g., APO-B, APO-E, lipoprotein lipase); cell-cell adhesion molecules (e.g., N-CAM, myelin-associated glycoprotein, selectins, pecam); angiogenin; lactoferrin; viral proteins (e.g., proteins from HIV, herpes complex) and other compounds which bind to GAG.

In some embodiments, methods described herein include a first screen for compounds that bind to Oct 3/4, Nanog, activated Stat-3, CXCR4, CD 133, SCA-1, Tra-1-60, CD 44, CD 73, CD 90, CD 105 and Stro-1. Compounds that are identified as binding to Oct 3/4, Nanog, activated Stat-3, CXCR4, CD 133, SCA-1, Tra-1-60, CD 44, CD 73, CD 90, CD 105 and Stro-1 can then be used in a second screen to identify those compounds that inhibit a function of Oct 3/4, Nanog, activated Stat-3, CXCR4, CD 133, SCA-1, Tra-1-60, CD 44, CD 73, CD 90, CD 105 and Stro-1. Alternatively, the first screen can be omitted and the compounds can simply be screened for their ability to inhibit a function of Oct 3/4, Nanog, activated Stat-3, CXCR4, CD 133, SCA-1, Tra-1-60, CD 44, CD 73, CD 90, CD 105 and Stro-1, e.g., to inhibit stem cell migration.

Once a compound that inhibits an action of Oct 3/4, Nanog, activated Stat-3, CXCR4, CD 133, SCA-1, Tra-1-60, CD 44, CD 73, CD 90, CD 105 and Stro-1 is identified, the compound can be considered a candidate compound that inhibits tumor formation, such as for example, inhibit cell proliferation, growth, stem cell migration and the like. The ability of such compounds to treat tumors can be evaluated in a population of viable cells or in an animal, e.g., an animal model.

Such compounds are useful, e.g., as candidate therapeutic compounds for the treatment of cancer. Thus, included herein are methods for screening for candidate therapeutic compounds for the treatment of, for example, cancer. The methods include administering the compound to a model of the condition, e.g., contacting a cell (in vitro) model with the compound, or administering the compound to an animal model of the condition, e.g., an animal model of a condition associated with decreased stem cell migration, such as cancer. The model is then evaluated for an effect of the candidate compound on the rate of migration in the model, and a candidate compound that decreases the rate of migration in the model can be considered a candidate therapeutic compound for the treatment of the condition. Such effects can include clinically relevant effects such as decreased tumor size or decreased tumor growth rate; decreased metastatic involvement or decreased rate of metastasis; decreased pain; increased life span; and so on. Such effects can be determined on a macroscopic or microscopic scale. Candidate therapeutic compounds identified by these methods can be further verified, e.g., by administration to human subjects in a clinical trial.

Test Compounds: The test compounds utilized in the assays and methods described herein can be, inter alia, nucleic acids, small molecules, organic or inorganic compounds, antibodies or antigen-binding fragments thereof, polynucleotides, peptides, or polypeptides. For example, Oct 3/4, Nanog, activated Stat-3, CXCR4, CD 133, SCA-1, Tra-1-60, CD 44, CD 73, CD 90, CD 105 polypeptides or polynucleotides (e.g., Oct 3/4, Nanog, activated Stat-3, CXCR4, CD 133, SCA-1, Tra-1-60, CD 44, CD 73, CD 90, CD 105 and Stro-1 polypeptide variants including truncation mutants, deletion mutants, and point mutants; nucleic acids including sense, antisense, aptamers, and small inhibitory RNAs (siRNAs) including short hairpin RNAs (shRNAs) and ribozymes) can be used as test compounds in the methods described herein. Alternatively, compounds or compositions that mimic the binding portions of Oct 3/4, Nanog, activated Stat-3, CXCR4, CD 133, SCA-1, Tra-1-60, CD 44, CD 73, CD 90, CD 105 and Stro-1 can be used. A test compound that has been screened by an in vitro method described herein and determined to have a desired activity, e.g., binding of compounds to Oct 3/4, Nanog, activated Stat-3, CXCR4, CD 133, SCA-1, Tra-1-60, CD 44, CD 73, CD 90, CD 105 and Stro-1 or affecting the functions of these molecules, for example, affecting growth, proliferation, migration of the stem cells to a tumor and the like, can be considered a candidate compound. A candidate compound that has been screened, e.g., in an in vitro or in vivo model, and determined to have a desirable effect on one or more inhibitory activities associated with treatment of cancer, (e.g. inhibition of proliferation, anti-angiogenic, apoptosis, decreased cell growth etc) can be considered a candidate therapeutic agent. Candidate therapeutic agents, once screened in a clinical setting, are therapeutic agents, and both types of agents can be optionally optimized (e.g., by derivatization), and formulated with pharmaceutically acceptable excipients or carriers to form pharmaceutical compositions.

Small Molecules: Small molecule test compounds can initially be members of an organic or inorganic chemical library. As used herein, “small molecules” refers to small organic or inorganic molecules of molecular weight below about 3,000 Daltons. The small molecules can be natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio., 1:60 (1997). In addition, a number of small molecule libraries are commercially available.

The test compound can have a structure that is based on an active fragment of Oct 3/4, Nanog, activated Stat-3, CXCR4, CD 133, SCA-1, Tra-1-60, CD 44, CD 73, CD 90, CD 105 and Stro-1. For example, computer modeling methods known in the can be used to rationally design a molecule that has a structure similar to an active fragment of Oct 3/4, Nanog, activated Stat-3, CXCR4, CD 133, SCA-1, Tra-1-60, CD 44, CD 73, CD 90, CD 105 and Stro-1 or portions thereof.

In some embodiments, the compounds are optimized to improve their therapeutic index, i.e., increase therapeutic efficacy and/or decrease unwanted side effects. Thus, in some embodiments, the methods described herein include optimizing the test or candidate compound. In some embodiments, the methods include formulating a therapeutic composition including a test or candidate compound (e.g., an optimized compound) and a pharmaceutically acceptable carrier. In some embodiments, the compounds are optimized by derivatization using methods known in the art.

Polynucleotides: In some embodiments, the test compound comprises a polynucleotide that encodes Oct 3/4, Nanog, activated Stat-3, CXCR4, CD 133, SCA-1, Tra-1-60, CD 44, CD 73, CD 90, CD 105 and Stro-1, or an active fragment thereof. In some embodiments, the compound is a polynucleotide that encodes an active fragment of Oct 3/4, Nanog, activated Stat-3, CXCR4, CD 133, SCA-1, Tra-1-60, CD 44, CD 73, CD 90, CD 105 and Stro-1 that retain ligand binding activity.

Sense Nucleic Acids: In some embodiments, the test compound comprises a polynucleotide that encodes a polypeptide that is at least about 85% identical to the amino acid sequence of Oct 3/4, Nanog, activated Stat-3, CXCR4, CD 133, SCA-1, Tra-1-60, CD 44, CD 73, CD 90, CD 105 and Stro-1. In some embodiments, the polynucleotide encodes a polypeptide that is at least about 90%, 95%, 99%, or 100% identical to the full length sequence of Oct 3/4, Nanog, activated Stat-3, CXCR4, CD 133, SCA-1, Tra-1-60, CD 44, CD 73, CD 90, CD 105 and Stro-1 or active fragments thereof. In some embodiments, the active fragment is at least about 10, 12, 14, 16, 18, 20, 22, 24, 26, 28, 30, 32, 34, 36, 38, 39, 40, 45, 50, 55, 60, 65, 70, 75, 80 or more amino acids long. The nucleic acid can include one or more noncoding regions of the coding strand of a nucleotide sequence encoding Oct 3/4, Nanog, activated Stat-3, CXCR4, CD 133, SCA-1, Tra-1-60, CD 44, CD 73, CD 90, CD 105 and Stro-1 (e.g., the 5′ and 3′ untranslated regions). A number of methods are known in the art for obtaining suitable nucleic acids, see, e.g., Sambrook et al., Molecular Cloning: A Laboratory Manual (Cold Spring Harbor Laboratory, Cold Spring Harbor, N.Y.; 3rd ed. 2001).

RNA Interference (RNAi): RNAi is a remarkably efficient process whereby double-stranded RNA (dsRNA, also referred to herein as siRNAs, for small interfering RNAs, or ds siRNAs, for double-stranded small interfering RNAs) induces the sequence-specific degradation of homologous mRNA in animals and plant cells (Hutvagner and Zamore, Curr. Opin. Genet. Dev., 12:225-232 (2002); Sharp, Genes Dev., 15:485-490 (2001)). In mammalian cells, RNAi can be triggered by duplexes of small interfering RNA (siRNA) (Chiu et al, Mol. Cell., 10:549-561 (2002); Elbashir et al., Nature, 411:494-498 (2001)), or by micro-RNAs (miRNA), functional small-hairpin RNA (shRNA), or other dsRNAs which are expressed in vivo using DNA templates with RNA polymerase III promoters (Zeng et al., Mol. Cell, 9:1327-1333 (2002); Paddison et al., Genes Dev., 16:948-958 (2002); Lee et al., Nature Biotechzol., 20:500-505 (2002); Paul et al., Nature Biotechnol., 20:505-508 (2002); Tuschl, T., Nature Biotechnol., 20:440-448 (2002); Yu et al., Proc. Natl. Acad. Sci. USA, 99(9):6047-6052 (2002); McManus et al., RNA, 8:842-850 (2002); Sui et al., Proc. Natl. Acad. Sci. USA, 99(6):5515-5520 (2002)).

The methods described herein can include the use of dsRNA molecules that are targeted to (i.e., bind to) Oct 3/4, Nanog, activated Stat-3, CXCR4, CD 133, SCA-1, Tra-1-60, CD 44, CD 73, CD 90, CD 105 and Stro-1 mRNA. Other preferred markers include, but not limited to: tenascins, proteoglycans, glycoproteins, glycolipids and other glycoconjugates that make up morphogenetic molecules and extracellular matrix molecules and their receptors, undulins and the like. Other non-limiting examples include polypeptide growth factors (e.g., FGFs1-9, PDGF, HGF, VEGF, TGF-β, IL-3); extracellular matrix components (e.g., laminins, fibronectins; thrombospondins, tenascins, collagens, VonWillebrand's factor); proteases and anti-proteases (e.g., thrombin, TPA, UPA, clotting factors IX and X, PAI-1); cell-adhesion molecules (e.g., N-CAM, LI, myelin-associated glycoprotein); proteins involved in lipoprotein metabolism (e.g., APO-B, APO-E, lipoprotein lipase); cell-cell adhesion molecules (e.g., N-CAM, myelin-associated glycoprotein, selectins, pecam); angiogenin; lactoferrin; viral proteins (e.g., proteins from HIV, herpes complex) and other compounds which bind to GAG.

The dsRNA molecules typically comprise 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is identical or substantially identical to the first strand. Each strand can also have one or more overhanging (i.e., non-complementary) nucleotides, e.g., one, two, three, four or more overhanging nucleotides, e.g., dTdTdT.

The dsRNA molecules can be chemically synthesized, or can be transcribed in vitro from a DNA template, or in vivo from, e.g., shRNA. The dsRNA molecules can be designed using any method known in the art; a number of algorithms are known in the art, see, e.g., Tuschl et al., Genes Dev 13(24):3191-7 (1999), and many are available on the internet, e.g., on the websites of Dharmacon (Lafayette, Colo.) or Ambion (Austin, Tex.).

Negative control siRNAs should have the same nucleotide composition as the selected siRNA, but without significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

micro RNA (miRNAs) of approximately 22 nucleotides can be used to regulate gene expression at the post transcriptional or translational level. miRNAs can be excised in the cell from an approximately 70 nucleotide precursor RNA stem-loop by Dicer, an RNase III-type enzyme, or a homolog thereof. By substituting the stem sequences of the miRNA precursor with miRNA sequence complementary to the target mRNA, a vector construct that expresses the novel miRNA can be used to produce siRNAs to initiate RNAi against specific mRNA targets in mammalian cells (Zeng (2002), supra). When expressed by DNA vectors containing polymerase III promoters, micro-RNA designed hairpins can silence gene expression (McManus (2002), supra).

dsRNA can be delivered directly into cells in vivo or in vitro using methods known in the art, e.g., cationic liposome transfection, nanoparticles, and electroporation, or expressed in vivo or in vitro from recombinant DNA constructs that allow longer-term target gene suppression in cells, including mammalian Pol III promoter systems (e.g., H1 or U6/snRNA promoter systems (Tuschl (2002), supra) capable of expressing functional double-stranded siRNAs; (Bagella et al., J. Cell. Physiol. 177:206-213 (1998); Lee et al. (2002), supra; Miyagishi et al. (2002), supra; Paul et al. (2002), supra; Yu et al. (2002), supra; Sui et al. (2002), supra).

Viral-mediated delivery mechanisms can also be used to induce specific silencing of targeted genes through expression of siRNA, for example, by generating recombinant adenoviruses harboring siRNA under RNA Pol II promoter transcription control (Xia et al. (2002), supra). Transcriptional termination by RNA Pol III occurs at runs of four consecutive T residues in the DNA template, providing a mechanism to end the siRNA transcript at a specific sequence. The dsRNA thus produced is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella et al. (1998), supra; Lee et al. (2002), supra; Miyagishi et al. (2002), supra; Paul et al. (2002), supra; Yu et al. (2002), supra; Sui et al. (2002) supra). Constructs containing siRNA sequence under the control of T7 promoter also make functional siRNAs when cotransfected into cells with a vector expression T7 RNA polymerase (Jacque (2002), supra).

In an animal, whole-embryo electroporation can efficiently deliver synthetic siRNA into post-implantation mouse embryos (Calegari et al., Proc. Natl. Acad. Sci. USA, 99(22):14236-40 (2002)). In adult mice, efficient delivery of siRNA can be accomplished by “high-pressure” delivery technique, a rapid injection (within 5 seconds) of a large volume of siRNA containing solution into animal via the tail vein (Liu (1999), supra; McCaffrey (2002), supra; Lewis, Nature Genetics 32:107-108 (2002)). Local delivery can also be used, e.g., with a carrier such as lipiodol (iodine in oil) to facilitate delivery into cells.

Engineered RNA precursors, introduced into cells or whole organisms as described herein, can be used for the production of a desired siRNA molecule. Such an siRNA molecule can then associate with endogenous protein components of the RNAi pathway to bind to and target a specific mRNA sequence for cleavage and destruction. In this fashion, the mRNA to be targeted by the siRNA generated from the engineered RNA precursor will be depleted from the cell or organism, leading to a decrease in the concentration of the protein encoded by that mRNA in the cell or organism. Additional information regarding the use of RNAi can be found in RNA Interference Editing, and Modification: Methods and Protocols (Methods in Molecular Biology), Gott, Ed. (Humana Press, 2004);

Antisense Polynucleotides: An “antisense” nucleic acid can include a nucleotide sequence that is complementary to a “sense” nucleic acid encoding a protein, e.g., complementary to all or a portion of the coding strand of a double-stranded cDNA molecule or complementary to Oct 3/4, Nanog, activated Stat-3, CXCR4, CD 133, SCA-1, Tra-1-60, CD 44, CD 73, CD 90, and CD 105 Stro-1, or ligands thereof, mRNA sequence. In another embodiment, the antisense nucleic acid molecule is antisense to a “noncoding region” of the coding strand of a nucleotide sequence encoding Oct 3/4, Nanog, activated Stat-3, CXCR4, CD 133, SCA-1, Tra-1-60, CD 44, CD 73, CD 90, and CD 105, Stro-1, or ligands thereof (e.g., the 5′ and 3′ untranslated regions). Other preferred markers include, but not limited to: tenascins, proteoglycans, glycoproteins, glycolipids and other glycoconjugates that make up morphogenetic molecules and extracellular matrix molecules and their receptors, undulins and the like. Other non-limiting examples include polypeptide growth factors (e.g., FGFs1-9, PDGF, HGF, VEGF, TGF-β, IL-3); extracellular matrix components (e.g., laminins, fibronectins; thrombospondins, tenascins, collagens, VonWillebrand's factor); proteases and anti-proteases (e.g., thrombin, TPA, UPA, clotting factors IX and X, PAI-1); cell-adhesion molecules (e.g., N-CAM, LI, myelin-associated glycoprotein); proteins involved in lipoprotein metabolism (e.g., APO-B, APO-E, lipoprotein lipase); cell-cell adhesion molecules (e.g., N-CAM, myelin-associated glycoprotein, selectins, pecam); angiogenin; lactoferrin; viral proteins (e.g., proteins from HIV, herpes complex) and other compounds which bind to GAG. An antisense polynucleotide statistically significantly inhibits the expression of the target gene.

Based upon the sequences disclosed herein, one of skill in the art can easily choose and synthesize any of a number of appropriate antisense molecules for use in accordance with the present invention. For example, a “gene walk” comprising a series of oligonucleotides of 15-30 nucleotides spanning the length of an Oct 3/4, Nanog, activated Stat-3, CXCR4, CD 133, SCA-1, Tra-1-60, CD 44, CD 73, CD 90, and CD 105, or ligands thereof, nucleic acid can be prepared, followed by testing for inhibition of Oct 3/4, Nanog, activated Stat-3, CXCR4, CD 133, SCA-1, Tra-1-60, CD 44, CD 73, CD 90, and CD 105 expression. Optionally, gaps of 5-10 nucleotides can be left between the oligonucleotides to reduce the number of oligonucleotides synthesized and tested. Other methods, including computational analysis, RNAse H mapping, and antisense-oligonucleotide scanning microarrays, can also be used (see, e.g., DNA Microarrays: A Practical Approach, Schena, Ed. (Oxford University Press 1999; Scherr and Rossi, Nuc. Acids Res., 26(22):5079-5085 (1998)).

An antisense nucleic acid can be designed such that it is complementary to the entire coding region of a target Oct 3/4, Nanog, activated Stat-3, CXCR4, CD 133, SCA-1, Tra-1-60, CD 44, CD 73, CD 90, and CD 105 mRNA, but can also be an oligonucleotide that is antisense to only a portion of the coding or noncoding region of the target mRNA. For example, the antisense oligonucleotide can be complementary to a region surrounding the translation start site of the target mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest).

Antisense nucleic acid molecules are typically administered to a subject (e.g., by direct injection at a tissue site), or generated in situ such that they hybridize with or bind to cellular mRNA and/or genomic DNA encoding an Oct 3/4, Nanog, activated Stat-3, CXCR4, CD. 133, SCA-1, Tra-1-60, CD 44, CD 73, CD 90, and CD 105, Stro-1 protein to thereby inhibit expression of the protein, e.g., by inhibiting transcription and/or translation. Other preferred molecules include, but not limited to: tenascins, proteoglycans, glycoproteins, glycolipids and other glycoconjugates that make up morphogenetic molecules and extracellular matrix molecules and their receptors, undulins and the like. Other non-limiting examples include polypeptide growth factors (e.g., FGFs1-9, PDGF, HGF, VEGF, TGF-β, IL-3); extracellular matrix components (e.g., laminins, fibronectins; thrombospondins, tenascins, collagens, VonWillebrand's factor); proteases and anti-proteases (e.g., thrombin, TPA, UPA, clotting factors IX and X, PAI-1); cell-adhesion molecules (e.g., N-CAM, LI, myelin-associated glycoprotein); proteins involved in lipoprotein metabolism (e.g., APO-B, APO-E, lipoprotein lipase); cell-cell adhesion molecules (e.g., N-CAM, myelin-associated glycoprotein, selectins, pecam); angiogenin; lactoferrin; viral proteins (e.g., proteins from HIV, herpes complex) and other compounds which bind to GAG.

Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter can be used.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al., Nucleic Acids. Res. 15:6625-6641 (1987)). The antisense nucleic acid molecule can also comprise a 2′-o-methylribonucleotide (Inoue et al. Nucleic Acids Res. 15:6131-6148 (1987)) or a chimeric RNA-DNA analogue (Inoue et al. FEBS Lett., 215:327-330 (1987)).

Oct 3/4, Nanog, activated Stat-3, CXCR4, CD 133, SCA-1, Tra-1-60, CD 44, CD 73, CD 90, CD 105, Stro-1 gene expression can be inhibited by targeting nucleotide sequences complementary to the regulatory regions (e.g., promoter and/or enhancer) to form triple helical structures that prevent transcription of the Oct 3/4, Nanog, activated Stat-3, CXCR4, CD 133, SCA-1, Tra-1-60, CD 44, CD 73, CD 90, CD 105, Stro-1 genes in target cells. Other preferred markers include, but not limited to: tenascins, proteoglycans, glycoproteins, glycolipids and other glycoconjugates that make up morphogenetic molecules and extracellular matrix molecules and their receptors, undulins and the like. Other preferred molecules include, but not limited to: tenascins, proteoglycans, glycoproteins, glycolipids and other glycoconjugates that make up morphogenetic molecules and extracellular matrix molecules and their receptors, undulins and the like. Other non-limiting examples include polypeptide growth factors (e.g., FGFs1-9, PDGF, HGF, VEGF, TGF-β, IL-3); extracellular matrix components (e.g., laminins, fibronectins; thrombospondins, tenascins, collagens, VonWillebrand's factor); proteases and anti-proteases (e.g., thrombin, TPA, UPA, clotting factors IX and X, PAI-1); cell-adhesion molecules (e.g., N-CAM, LI, myelin-associated glycoprotein); proteins involved in lipoprotein metabolism (e.g., APO-B, APO-E, lipoprotein lipase); cell-cell adhesion molecules (e.g., N-CAM, myelin-associated glycoprotein, selectins, pecam); angiogenin; lactoferrin; viral proteins (e.g., proteins from HIV, herpes complex) and other compounds which bind to GAG. See generally, Helene, Annticancer Drug Des. 6:569-84 (1991); Helene, Ann. N.Y. Acad. Sci. 660:27-36 (1992); and Maher, gBioassays 14:807-15 (1992).

The potential sequences that can be targeted for triple helix formation can be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

Ribozymes: Ribozymes are a type of RNA that can be engineered to enzymatically cleave and inactivate other RNA targets in a specific, sequence-dependent fashion. By cleaving the target RNA, ribozymes inhibit translation, thus preventing the expression of the target gene. Ribozymes can be chemically synthesized in the laboratory and structurally modified to increase their stability and catalytic activity using methods known in the art. Alternatively, ribozyme genes can be introduced into cells through gene-delivery mechanisms known in the art. A ribozyme having specificity for an Oct 3/4, Nanog, activated Stat-3, CXCR4, CD 133, SCA-1, Tra-1-60, CD 44, CD 73, CD 90, CD 105, Stro-1-encoding nucleic acid can include one or more sequences complementary to the nucleotide sequence of an Oct 3/4, Nanog, activated Stat-3, CXCR4, CD 133, SCA-1, Tra-1-60, CD 44, CD 73, CD 90, Stro-1 and CD 105 cDNA, and a sequence having known catalytic sequence responsible for mRNA cleavage (see U.S. Pat. No. 5,093,246 or Haselhoff and Gerlach, Nature, 334:585-591 (1988)). For example, a derivative of a Tetrahymena L-19 IVS RNA can be constructed in which the nucleotide sequence of the active site is complementary to the nucleotide sequence to be cleaved in an Oct 3/4, Nanog, activated Stat-3, CXCR4, CD 133, SCA-1, Tra-1-60, CD 44, CD 73, CD 90, Stro-1 and CD 105-encoding mRNA. See, e.g., Cech et al., U.S. Pat. No. 4,987,071; and Cech et al., U.S. Pat. No. 5,116,742. Alternatively, Oct 3/4, Nanog, activated Stat-3, CXCR4, CD 133, SCA-1, Tra-1-60, CD 44, CD 73, CD 90, Stro-1, and CD 105 mRNA can be used to select a catalytic RNA having a specific ribonuclease activity from a pool of RNA molecules. See, e.g., Bartel, and Szostak, Science, 261:1411-1418 (1993).

Peptides/Polypeptides: In some embodiments, the test compound includes an Oct 3/4, Nanog, activated Stat-3, CXCR4, CD 133, SCA-1, Tra-1-60, CD 44, CD 73, CD 90, Stro-1, and CD 105 polypeptides, or an active fragments thereof. In some embodiments, the compound is an active fragment of Oct 3/4, Nanog, activated Stat-3, CXCR4, CD 133, SCA-1, Tra-1-60, CD 44, CD 73, CD 90, and CD 105 that retains ligand binding activity. Other preferred molecules include, but not limited to: tenascins, proteoglycans, glycoproteins, glycolipids and other glycoconjugates that make up morphogenetic molecules and extracellular matrix molecules and their receptors, undulins and the like. Other non-limiting examples include polypeptide growth factors (e.g., FGFs1-9, PDGF, HGF, VEGF, TGF-β, IL-3); extracellular matrix components (e.g., laminins, fibronectins; thrombospondins, tenascins, collagens, VonWillebrand's factor); proteases and anti-proteases (e.g., thrombin, TPA, UPA, clotting factors IX and X, PAI-1); cell-adhesion molecules (e.g., N-CAM, LI, myelin-associated glycoprotein); proteins involved in lipoprotein metabolism (e.g., APO-B, APO-E, lipoprotein lipase); cell-cell adhesion molecules (e.g., N-CAM, myelin-associated glycoprotein, selectins, pecam); angiogenin; lactoferrin; viral proteins (e.g., proteins from HIV, herpes complex) and other compounds which bind to GAG.

In some embodiments, the test compound comprises a polypeptide that is at least about 85% identical to the amino acid sequence of Oct 3/4, Nanog, activated Stat-3, CXCR4, CD 133, SCA-1, Tra-1-60, CD 44, CD 73, CD 90, Stro-1 and CD 105. In some embodiments, the polypeptide is at least about 90%, 95%, 99%, or 100% identical to the full length sequence of Oct 3/4, Nanog, activated Stat-3, CXCR4, CD 133, SCA-1, Tra-1-60, CD 44, CD 73, CD 90, Stro-1 and CD 105, or an active fragment thereof. In some embodiments, the active fragment is at least about 10, 12, 14, 16, 18, 20, 22, 24, 26, 28, 30, 32, 34, 36, 38, 39, 40, 45, 50, 55, 60, 65, 70, 75, 80 or more amino acids long. A “polypeptide comprising an active fragment of Oct 3/4, Nanog, activated Stat-3, CXCR4, CD 133, SCA-1, Tra-1-60, CD 44, CD 73, CD 90, Stro-1 or CD 105” includes less than the full length of each, but can include other (i.e., non-Oct 3/4, Nanog, activated Stat-3, CXCR4, CD 133, SCA-1, Tra-1-60, CD 44, CD 73, CD 90, Stro-1 and CD 105) proteins or fragments thereof, e.g., fluorescent proteins such as green fluorescent protein (GFP), red fluorescent protein (RFP), blue fluorescent protein (BFP) or yellow fluorescent protein (YFP), or peptides that enhance delivery, e.g., a TAT protein transduction domain (PTD).

### Pharmaceutical Compositions and Methods of Administration

The invention includes compounds that mimic an action of Oct 3/4, Nanog, activated Stat-3, CXCR4, CD 133, SCA-1, Tra-1-60, CD 44, CD 73, CD 90, and CD 105, identified by a method described herein. Other preferred molecules include, but not limited to: tenascins, proteoglycans, glycoproteins, glycolipids and other glycoconjugates that make up morphogenetic molecules and extracellular matrix molecules and their receptors, undulins and the like. Other non-limiting examples include polypeptide growth factors (e.g., FGFs1-9, PDGF, HGF, VEGF, TGF-β, IL-3); extracellular matrix components (e.g., laminins, fibronectins; thrombospondins, tenascins, collagens, VonWillebrand's factor); proteases and anti-proteases (e.g., thrombin, TPA, UPA, clotting factors IX and X, PAI-1); cell-adhesion molecules (e.g., N-CAM, LI, myelin-associated glycoprotein); proteins involved in lipoprotein metabolism (e.g., APO-B, APO-E, lipoprotein lipase); cell-cell adhesion molecules (e.g., N-CAM, myelin-associated glycoprotein, selectins, pecam); angiogenin; lactoferrin; viral proteins (e.g., proteins from HIV, herpes complex) and other compounds which bind to GAG.

In some embodiments, the compound is a protein, nucleic acid, small molecule, peptide, siRNA, ribozyme, antisense oligonucleotide or antibody, e.g., that binds specifically to an Oct 3/4, Nanog, activated Stat-3, CXCR4, CD 133, SCA-1, Tra-1-60, CD 44, CD 73, CD 90, Stro-1 and CD 105 nucleic acid or protein and inhibits Oct 3/4, Nanog, activated Stat-3, CXCR4, CD 133, SCA-1, Tra-1-60, CD 44, CD 73, CD 90, Stro-1, and CD 105 function, e.g., by degrading Oct 3/4, Nanog, activated Stat-3, CXCR4, CD 133, SCA-1, Tra-1-60, CD 44, CD 73, CD 90, Stro-1 and CD 105.

Methods of Formulation: The compounds described herein can be incorporated into pharmaceutical compositions. Such compositions typically include the active ingredient and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from pressured container or dispenser which contains a suitable propellant, e.g., a gas such as carbon dioxide, or a nebulizer. Such methods include those described in U.S. Pat. No. 6,468,798. Compositions for inhalation can also include propellants, surfactants, and other additives, e.g., to improve dispersion, flow, and bioavailability.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Compounds comprising nucleic acids can also be administered by transfection or infection using methods known in the art, including but not limited to the methods described in McCaffrey et al. (2002), Nature, 418(6893), 38-9 (hydrodynamic transfection); Xia et al. (2002), Nature Biotechnol., 20(10), 1006-10 (viral-mediated delivery); or Putnam (1996), Am. J Health Syst. Pharm., 53(2), 151-160, erratum at Am. J. Health Syst. Pharm., 53(3), 325 (1996). Compounds comprising nucleic acids can also be administered by method suitable for administration of DNA vaccines. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88(2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the compounds are prepared with carriers that will protect the active ingredient against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In some embodiments, the compounds (e.g., polypeptides) are modified to enhance delivery into cells, e.g., by the addition of an optimized or native TAT protein transduction domain (PTD), e.g., as described in Ho et al., Cancer Res. 61(2):474-7 (2001). Where the compound is a polypeptide, the polypeptide can be a fusion protein comprising an active portion (e.g., an active fragment of Apoptin) and a TAT PTD fused in frame.

The pharmaceutical compositions can be included in a container, pack, or dispenser together with instructions for administration.

Methods of Treatment: As used herein, the term “treatment” is defined as the application or administration of a therapeutic agent described herein, or identified by a method described herein, to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

Examples of routes of administration include parenteral, e.g., intravenous, intradermal, subcutaneous, oral (e.g., inhalation), transdermal (topical), transmucosal, and rectal administration.

Therapeutic agents include, for example, proteins, nucleic acids, small molecules, peptides, antibodies, siRNAs, ribozymes, and antisense oligonucleotides. Dosage, toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a compound (i.e., an effective dosage) means an amount sufficient to produce a therapeutically (e.g., clinically) desirable result; the exact nature of the result will vary depending on the nature of the disorder being treated. For example, where the disorder to be treated iscancer, the result can be a cessation of abnormal cell growth, proliferation etc. Where the disorder is associated with increased cellular proliferation, the result can be a cessation of or decrease in cellular proliferation. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

The present invention provides for both prophylactic and therapeutic methods of treating a subject at risk of (or susceptible to) a disorder or having a disorder associated with cancer.

Cellular Proliferative Disorders: Compounds that inhibit Oct 3/4, Nanog, activated Stat-3, CXCR4, CD 133, SCA-1, Tra-1-60, CD 44, CD 73, CD 90, Stro-1 and CD 105 are useful in the treatment of disorders associated with cancer, e.g., cellular proliferative disorders or cellular differentiative disorders, e.g., by inducing apoptosis in those cells. Other preferred molecules include, but not limited to: tenascins, proteoglycans, glycoproteins, glycolipids and other glycoconjugates that make up morphogenetic molecules and extracellular matrix molecules and their receptors, undulins and the like. Other non-limiting examples include polypeptide growth factors (e.g., FGFs1-9, PDGF, HGF, VEGF, TGF-β, IL-3); extracellular matrix components (e.g., laminins, fibronectins; thrombospondins, tenascins, collagens, VonWillebrand's factor); proteases and anti-proteases (e.g., thrombin, TPA, UPA, clotting factors IX and X, PAI-1); cell-adhesion molecules (e.g., N-CAM, LI, myelin-associated glycoprotein); proteins involved in lipoprotein metabolism (e.g., APO-B, APO-E, lipoprotein lipase); cell-cell adhesion molecules (e.g., N-CAM, myelin-associated glycoprotein, selectins, pecam); angiogenin; lactoferrin; viral proteins (e.g., proteins from HIV, herpes complex) and other compounds which bind to GAG.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the term “hyperproliferative,” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. In some embodiments, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, Crit. Rev. in Oncol./Hemotol., 11:267-97 (1991)); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

Other examples of proliferative and/or differentiative disorders include skin disorders. The skin disorder may involve the aberrant activity of a cell or a group of cells or layers in the dermal, epidermal, or hypodermal layer, or an abnormality in the dermal-epidermal junction.

Examples of skin disorders include psoriasis, psoriatic arthritis, dermatitis (eczema), e.g., exfoliative dermatitis or atopic dermatitis, pityriasis rubra pilaris, pityriasis rosacea, parapsoriasis, pityriasis lichenoiders, lichen planus, lichen nitidus, ichthyosiform dermatosis, keratodermas, dermatosis, alopecia greata, pyoderma gangrenosum, vitiligo, pemphigoid (e.g., ocular cicatricial pemphigoid or bullous pemphigoid), urticaria, prokeratosis, rheumatoid arthritis that involves hyperproliferation and inflammation of epithelial-related cells lining the joint capsule; dermatitises such as atopic dennatitis, allergic dermatitis, seborrheic dermatitis or solar dermatitis; keratoses such as seborrheic keratosis, senile keratosis, actinic keratosis, photo-induced keratosis, and keratosis follicularis; acne vulgaris; keloids and prophylaxis against keloid formation; nevi; warts including verruca, condyloma or condyloma acuminatum, and human papilloma viral (HPV) infections such as venereal warts; leukoplakia; lichen planus; and keratitis.

Diagnosis and Detection: In diagnosis and detection of the stem cells, biopsies from tumors in subjects are tested for the identity of Oct 3/4, Nanog, Stro-1 (used as illustrative examples) and allelic variants of these or other genes identified according to the invention. The polynucleotides encoding Oct 3/4, Nanog, Stro-1 and allelic variants thereof may be used for diagnostic purposes. A variety of protocols for measuring Oct 3/4, Nanog, Stro-1 levels; including ELISAs, RIAs, and FACS, are known in the art and provide a basis for detecting genes expressing Oct 3/4, Nanog, Stro-1.

Other diagnostic methods include use of polynucleotides in a variety of methods. The polynucleotides which may be used include oligonucleotide sequences, complementary RNA and DNA molecules, and PNAs. The polynucleotides may be used to detect and quantitate gene expression in biopsied tissues in which expression of Oct 3/4, Nanog, Stro-1 may be correlated with the number of stem cells present in the bulk tumor. The diagnostic assay may be used to determine absence, presence, and excess expression of Oct 3/4, Nanog, Stro-1, and to monitor regulation of Oct 3/4, Nanog, Stro-1 levels during therapeutic intervention.

One suitable method for diagnosis and candidate drug discovery includes contacting a test sample with any one of the genes expressing Oct 3/4, Nanog, Stro-1, an allele or fragment thereof; and detecting interaction of the test sample with the Oct 3/4, Nanog, Stro-1 expressing genes, an allele or fragment thereof. The test sample is a mammalian tissue or fluid (e.g. blood) sample. The Oct 3/4, Nanog, Stro-1 expressing gene, an allele or fragment thereof, can be detectably labeled e.g. with a fluorescent or radioactive component.

In one aspect, hybridization with oligonucleotide probes that are capable of detecting polynucleotide sequences, including genomic sequences, encoding Oct 3/4, Nanog, Stro-1 or closely related molecules may be used to identify nucleic acid sequences which encode Oct 3/4, Nanog, Stro-1. The specificity of the probe, whether it is made from a highly specific region, e.g., the 5′ regulatory region, or from a less specific region, e.g., a conserved motif, and the stringency of the hybridization or amplification (maximal, high, intermediate, or low), will determine whether the probe identifies only naturally occurring sequences encoding Oct 3/4, Nanog, Stro-1, allelic variants, or related sequences.

Probes may also be used for the detection of related sequences, and should preferably have at least 50% sequence identity or homology to any of the Oct 3/4, Nanog, Stro-1 encoding sequences, more preferably at least about 60, 70, 75, 80, 85, 90 or 95 percent sequence identity to any of the Oct 3/4, Nanog, Stro-1 encoding sequences (sequence identity determinations discussed above, including use of BLAST program). The hybridization probes of the subject invention may be DNA or RNA and may be derived from the sequences of the invention or from genomic sequences including promoters, enhancers, and introns of the Oct 3/4, Nanog, Stro-1 genes.

“Homologous”, as used herein, refers to the subunit sequence similarity between two polymeric molecules, e.g., between two nucleic acid molecules such as two DNA molecules, or two polypeptide molecules. When a subunit position in both of the two molecules is occupied by the same monomeric subunit (e.g., if a position in each of two DNA molecules is occupied by adenine) then they are homologous at that position. The homology between two sequences is a direct function of the number of matching or homologous positions. For example, if 5 of 10 positions in two compound sequences are matched or homologous then the two sequences are 50% homologous, if 9 of 10 are matched or homologous, the two sequences share 90% homology. By way of example, the DNA sequences 3′ ATTGCC 5′ and 3′ TTTCCG 5′ share 50% homology.

Means for producing specific hybridization probes for DNAs encoding Oct 3/4, Nanog, Stro-1 include the cloning of polynucleotide sequences encoding Oct 3/4, Nanog, Stro-1 or derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³²S, or by enzymatic labels, such as alkaline phosphatase coupled to the probe via avidin-biotin coupling systems, fluorescent labeling, and the like.

The polynucleotide sequences encoding Oct 3/4, Nanog, Stro-1 may be used in Southern or Northern analysis, dot blot, or other membrane-based technologies; in PCR technologies; in dipstick, pin, and multiformat ELISA-like assays; and in microarrays utilizing fluids or tissues from patients to detect altered Oct 3/4, Nanog, Stro-1 expression. Gel-based mobility-shift analyses may be employed. Other suitable qualitative or quantitative methods are well known in the art.

Identity of genes, or variants thereof, can be verified using techniques well known in the art. Examples include but are not limited to, nucleic acid sequencing of amplified genes, hybridization techniques such as single nucleic acid polymorphism analysis (SNP), microarrays wherein the molecule of interest is immobilized on a biochip. Overlapping cDNA clones can be sequenced by the dideoxy chain reaction using fluorescent dye terminators and an ABI sequencer (Applied Biosystems, Foster City, Calif.). Any type of assay wherein one component is immobilized may be carried out using the substrate platforms of the invention. Bioassays utilizing an immobilized component are well known in the art. Examples of assays utilizing an immobilized component include for example, immunoassays, analysis of protein-protein interactions, analysis of protein-nucleic acid interactions, analysis of nucleic acid-nucleic acid interactions, receptor binding assays, enzyme assays, phosphorylation assays, diagnostic assays for determination of disease state, genetic profiling for drug compatibility analysis, SNP detection, etc.

An Oct 3/4, Nanog, Stro-1 expressing gene or Oct 3/4, Nanog, Stro-1 expressing related gene means the gene and all currently known variants thereof, including the different mRNA transcripts to which the gene and its variants can give rise, and any further gene variants which may be elucidated. In general, however, such variants will have significant homology (sequence identity) to a sequence of Oct 3/4, Nanog, Stro-1 e.g. a variant will have at least about 70 percent homology (sequence identity) to a sequence of Oct 3/4, Nanog, Stro-1, more typically at least about 75, 80, 85, 90, 95, 97, 98 or 99 homology (sequence identity) to a sequence of Oct 3/4, Nanog, Stro-1. Homology of a variant can be determined by any of a number of standard techniques such as a BLAST program.

Identification of a nucleic acid sequence capable of binding to a biomolecule of interest can be achieved by immobilizing a library of nucleic acids onto the substrate surface so that each unique nucleic acid was located at a defined position to form an array. The array would then be exposed to the biomolecule under conditions which favored binding of the biomolecule to the nucleic acids. Non-specifically binding biomolecules could be washed away using mild to stringent buffer conditions depending on the level of specificity of binding desired. The nucleic acid array would then be analyzed to determine which nucleic acid sequences bound to the biomolecule. Preferably the biomolecules would carry a fluorescent tag for use in detection of the location of the bound nucleic acids.

An assay using an immobilized array of nucleic acid sequences may be used for determining the sequence of an unknown nucleic acid; single nucleotide polymorphism (SNP) analysis; analysis of gene expression patterns from a particular species, tissue, cell type, etc.; gene identification; etc.

Additional diagnostic uses for oligonucleotides designed from the sequences encoding Oct 3/4, Nanog, Stro-1 may involve the use of PCR, as described in detail in the Examples which follow. These oligomers may be chemically synthesized, generated enzymatically, or produced in vitro. Oligomers will preferably contain a fragment of a polynucleotide encoding Oct 3/4, Nanog, Stro-1, or a fragment of a polynucleotide complementary to the polynucleotide encoding Oct 3/4, Nanog, Stro-1, and will be employed under optimized conditions for identification of a specific gene. Oligomers may also be employed under less stringent conditions for detection or quantitation of closely-related DNA or RNA sequences.

High stringency conditions are known in the art; see for example Maniatis et al., Molecular Cloning: A Laboratory Manual, 2d Edition, 1989, and Short Protocols in Molecular Biology, ed. Ausubel, et al., both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH and nucleic acid concentration) at which 50% of a nucleic acid sequence complementary to the target, hybridizes to the target sequence at equilibrium. Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short nucleic acid sequences (e.g. 10 to 50 nucleotides) and at least about 60° C. for long nucleic acid sequences (e.g. greater than 50 nucleotides). Stringent conditions may also be achieved with the addition of destabilizing agents such as formamide.

The phrase “stringent hybridization” is used herein to refer to conditions under which polynucleic acid hybrids are stable. As known to those of skill in the art, the stability of hybrids is reflected in the melting temperature (Tₘ) of the hybrids. In general, the stability of a hybrid is a function of sodium ion concentration and temperature. Typically, the hybridization reaction is performed under conditions of lower stringency, followed by washes of varying, but higher, stringency. Reference to hybridization stringency relates to such washing conditions. As used herein, the phrase “moderately stringent hybridization” refers to conditions that permit target-DNA to bind a complementary nucleic acid that has about 60% identity, preferably about 75% identity, more preferably about 85% identity to the target DNA; with greater than about 90% identity to target-DNA being especially preferred. Preferably, moderately stringent conditions are conditions equivalent to hybridization in 50% formamide, 5× Denhart's solution, 5×SSPE, 0.2% SDS at 42° C., followed by washing in 0.2×SSPE, 0.2% SDS, at 65° C.

The phrase “high stringency hybridization” refers to conditions that permit hybridization of only those nucleic acid sequences that form stable hybrids in, for example, 0.018 M NaCl at 65° C. (i.e., if a hybrid is not stable in 0.018 M NaCl at 65° C., it will not be stable under high stringency conditions). High stringency conditions can be provided, for example, by hybridization in 50% formamide, 5× Denhart's solution, 5×SSPE, 0.2% SDS at 42° C., followed by washing in 0.1×SSPE, and 0.1% SDS at 65° C.

The phrase “low stringency hybridization” refers to conditions equivalent to hybridization in 10% formamide, 5× Denhart's solution, 6×SSPE, 0.2% SDS at 42° C., followed by washing in 1×SSPE, 0.2% SDS, at 50° C. Denhart's solution and SSPE (see, e.g., Sambrook et al., Molecular Cloning, A Laboratory Manual, Cold Spring Harbor Laboratory Press, 1989) are well known to those of skill in the art as are other suitable hybridization buffers.

In further embodiments, oligonucleotides or longer fragments derived from Oct 3/4, Nanog, Stro-1 polynucleotide sequences, may be used as targets in a microarray. The microarray can be used to monitor the identity and/or expression level of large numbers of genes and gene transcripts simultaneously to identify genes with which Oct 3/4, Nanog, Stro-1 or its product interacts and/or to assess the efficacy of candidate therapeutic agents in regulating genes that modulate Oct 3/4, Nanog, Stro-1 expression in tumors. Microarrays may be used to particular advantage in diagnostic assays, to identify genetic variants, mutations, and polymorphisms of genes that express Oct 3/4, Nanog, Stro-1 in a biological sample from a mammal, such as a human or other research subject or clinical patient. This information may be used to determine gene function, and to develop and monitor the activities of therapeutic agents.

In other embodiments, oligonucleotides or longer fragments derived from any of the polynucleotide sequences herein (including, in non-limiting fashion, human sequences) and genomic sequences adjacent to them may be used as diagnostic reagents, such as to detect single-nucleotide polymorphisms or other variations or mutations in genes expressing Oct 3/4, Nanog, Stro-1 or a homologous gene, amplification of Oct 3/4, Nanog, Stro-1 or homologous nucleic acid sequences, and for use in nucleic acid sequencing methods.

Microarrays may be prepared, used, and analyzed using methods known in the art (see, e.g., Brennan et al., 1995, U.S. Pat. No. 5,474,796; Schena et al., 1996, Proc. Natl. Acad. Sci. U.S.A. 93: 10614-10619; Baldeschweiler et al., 1995, PCT application WO95/251116; Shalon, et al., 1995, PCT application WO95/35505; Heller et al., 1997, Proc. Natl. Acad. Sci. U.S.A. 94: 2150-2155; and Heller et al., 1997, U.S. Pat. No. 5,605,662).

Expression or activity levels for Oct 3/4, Nanog, Stro-1 also may be examined. Normal or standard values for Oct 3/4, Nanog, Stro-1 expression are established by combining body fluids or cell extracts taken from normal mammalian subjects, preferably human, with antibody to Oct 3/4, Nanog, Stro-1 under conditions suitable for complex formation. The amount of standard complex formation may be quantitated by various methods, preferably by photometric means. Quantities of Oct 3/4, Nanog, Stro-1 expressed in subject, control, and disease samples from biopsied tissues are compared with the standard values. Deviation between standard and subject values establishes the parameters for Oct 3/4, Nanog, Stro-1 expression. Parameters studied include, but are not limited to, the below and those described throughout the specification:

Candidate agents include numerous chemical classes, though typically they are organic compounds including small organic compounds, nucleic acids including oligonucleotides, and peptides. Small organic compounds suitably may have e.g. a molecular weight of more than about 40 or 50 yet less than about 2,500. Candidate agents may comprise functional chemical groups that interact with proteins and/or DNA.

Candidate agents may be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of e.g. bacterial, fungal and animal extracts are available or readily produced.

Therapeutic agent assays of the invention suitably include, animal models, cell-based systems and non-cell based systems.

Preferably, genes expressing Oct 3/4, Nanog, Stro-1, variants, fragments, or oligopeptides thereof are used for identifying agents of therapeutic interest, e.g. by screening libraries of compounds or otherwise identifying compounds of interest by any of a variety of drug screening or analysis techniques. Preferably, the Oct 3/4, Nanog, Stro-1 are amino acids and may be free in solution, affixed to a solid support, borne on a cell surface, or located intracellularly. The formation of binding complexes between Oct 3/4, Nanog, Stro-1 and the agent being tested may be measured and then tested.

Another technique for drug screening provides for high throughput screening of compounds having suitable binding affinity to the protein of interest (see, e.g., Geysen et al., 1984, PCT application WO84/03564). In this method, large numbers of different small test compounds are synthesized on a solid substrate. The test compounds are reacted with Oct 3/4, Nanog, Stro-1, or fragments thereof, and washed. Bound Oct 3/4, Nanog, Stro-1 is then detected by methods well known in the art. Purified Oct 3/4, Nanog, Stro-1 can also be coated directly onto plates for use in the aforementioned drug screening techniques. Alternatively, non-neutralizing antibodies can be used to capture the peptide and immobilize it on a solid support.

### Immunotherapeutics

In a preferred embodiment, antibodies directed to Oct 3/4, Nanog, Stro-1 are covalently bound or fused to therapeutic effector domains. Examples of therapeutic effector domains include, but not limited to: endostatin, angiogenin, angiostatin, chemokines, angioarrestin, angiostatin (plasminogen fragment), basement-membrane collagen-derived anti-angiogenic factors (tumstatin, canstatin, or arrestin), anti-angiogenic antithrombin III, cartilage-derived inhibitor (CDI), CD59 complement fragment, fibronectin fragment, gro-beta, heparinases, heparin hexasaccharide fragment, human chorionic gonadotropin (hCG), interferon alpha/beta/gamma, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-b), vasculostatin, vasostatin (calreticulin fragment) and the like.

Cytolytic molecules that can be used to fuse to an antibody or fragment thereof, include, but are not limited to TNF-α, TNF-β, suitable effector genes such as those that encode a peptide toxin—such as ricin, abrin, diphtheria, gelonin, Pseudomonas exotoxin A, Crotalus durissus terrificus toxin, Crotalus adamenteus toxin, Naja naja toxin, and Naja mocambique toxin. (Hughes et al., Hum. Exp. Toxicol. 15:443, 1996; Rosenblum et al., Cancer Immunol. Immunother. 42:115, 1996; Rodriguez et al., Prostate 34:259, 1998; Mauceri et al., Cancer Res. 56:4311; 1996).

Also suitable are genes that induce or mediate apoptosis—such as the ICE-family of cysteine proteases, the Bcl-2 family of proteins, Bax, bclXs and caspases (Favrot et al., Gene Ther. 5:728, 1998; McGill et al., Front. Biosci. 2:D353, 1997; McDonnell et al., Semin. Cancer Biol. 6:53, 1995). Another potential anti-tumor agent is apoptin, a protein that induces apoptosis even where small drug chemotherapeutics fail (Pietersen et al., Adv. Exp. Med. Biol. 465:153, 2000). Koga et al. (Hu. Gene Ther. 11:1397, 2000) propose a telomerase-specific gene therapy using the hTERT gene promoter linked to the apoptosis gene Caspase-8 (FLICE).

Also of interest are enzymes present in the lytic package that cytotoxic T lymphocytes or LAK cells deliver to their targets. Perforin, a pore-forming protein, and Fas ligand are major cytolytic molecules in these cells (Brandau et al., Clin. Cancer Res. 6:3729, 2000; Cruz et al., Br. J. Cancer 81:881, 1999). CTLs also express a family of at least 11 serine proteases termed granzymes, which have four primary substrate specificities (Kam et al., Biochim. Biophys. Acta 1477:307, 2000). Low concentrations of streptolysin 0 and pneumolysin facilitate granzyme B-dependent apoptosis (Browne et al., Mol. Cell. Biol. 19:8604, 1999).

Other suitable effectors encode polypeptides having activity that is not itself toxic to a cell, but renders the cell sensitive to an otherwise nontoxic compound—either by metabolically altering the cell, or by changing a non-toxic prodrug into a lethal drug. Exemplary is thymidine kinase (tk), such as may be derived from a herpes simplex virus, and catalytically equivalent variants. The HSV tk converts the anti-herpetic agent ganciclovir (GCV) to a toxic product that interferes with DNA replication in proliferating cells.

If desired, although not required, factors may also be included, such as, but not limited to, interleukins, e.g. IL-2, IL-3, IL-6, and IL-11, as well as the other interleukins, the colony stimulating factors, such as GM-CSF, interferons, e.g. γ-interferon, erythropoietin.

In another preferred embodiment, the invention provides for antibody fusion molecules comprising a modulatory or cytotoxic molecule fused to the F_{c} region, C_{H}1, C_{H}2 and/or C_{H}3, Fab, Fab′, F(ab′)₂, single chain Fv (S_{c}Fv) and Fv fragments, as well as any portion of an antibody having specificity toward a desired target epitope or epitopes. Also preferred are antibodies or antibody fragments or to single chain, two-chain, and multi-chain proteins and glycoproteins belonging to the classes of polyclonal, monoclonal, chimeric, bispecific and hetero immunoglobulins (monoclonal antibodies being preferred); it also includes synthetic and genetically engineered variants of these immunoglobulins.

In another preferred embodiment, carrier domains within the invention can be used to introduce an effector function to the molecule. For introducing an effector function to the molecule, the carrier domain can be a protein that has been shown to possess cytotoxic or immune response-stimulating properties. For instance, carrier domains for introducing a cytotoxic function to the molecule include a bacterial toxin, ricin, abrin, saporin, pokeweed viral protein, and constant region domains from an immunoglobulin molecule (e.g., for antibody dependent cell-mediated cytotoxicity). Molecules that contain a cytotoxic carrier domain can be used to selectively kill cells.

For introducing immune response-stimulating properties to a molecule, carrier domains within the invention include any molecule known to activate an immune system component. For example, antibodies and antibody fragments (e.g., CH₂—CH₃) can be used as a carrier domain to engage Fc receptors or to activate complement components. A number of other immune system-activating molecules are known that might also be used as a carrier domain, e.g., microbial superantigens, adjuvant components, lipopolysaccharide (LPS), and lectins with mitogenic activity. Other carrier domains that can be used to introduce an effector function to the molecule can be identified using known methods. For instance, a molecule can be screened for suitability as a carrier domain by fusing the molecule to an anti-angiogenic agent and testing the molecule in in vitro or in vivo cell cytotoxicity and humoral response assays.

In another preferred embodiment, isolated, enriched cultures of stem cells are used to vaccinate or stimulate an immune response against cells expressing any one of the markers described herein. Examples of antigens include: Oct 3/4, Nanog, activated Stat-3, CXCR4, CD 133, SCA-1, Tra-1-60, CD 44, CD 73, CD 90, CD 105 and Stro-1.

“Immune cells” as used herein, is meant to include any cells of the immune system that may be assayed, including, but not limited to, B lymphocytes, also called B cells, T lymphocytes, also called T cells, natural killer (NK) cells, lymphokine-activated killer (LAK) cells, monocytes, macrophages, neutrophils, granulocytes, mast cells, platelets, Langerhan's cells, stem cells, dendritic cells, peripheral blood mononuclear cells, tumor-infiltrating (TIL) cells, gene modified immune cells including hybridomas, drug modified immune cells, and derivatives, precursors or progenitors of the above cell types.

“Activity”, “activation” “stimulation” or “augmentation” is the ability of immune cells to respond and exhibit, on a measurable level, an immune function. Measuring the degree of activation refers to a quantitative assessment of the capacity of immune cells to express enhanced activity when further stimulated as a result of prior activation. The enhanced capacity may result from biochemical changes occurring during the activation process that allow the immune cells to be stimulated to activity in response to low doses of stimulants.

Immune cell activity that may be measured include, but is not limited to: (1) cell proliferation by measuring the DNA replication; (2) enhanced cytokine production, including specific measurements for cytokines, such as IFN-γ, GM-CSF, or TNF-α; (3) cell mediated target killing or lysis; (4) cell differentiation; (5) immunoglobulin production; (6) phenotypic changes; (7) production of chemotactic factors or chemotaxis, meaning the ability to respond to a chemotactin with chemotaxis; (8) immunosuppression, by inhibition of the activity of some other immune cell type; and, (9) apoptosis, which refers to fragmentation of activated immune cells under certain circumstances, as an indication of abnormal activation.

In preferred embodiments, proteins, peptides of Oct 3/4, Nanog, activated Stat-3, CXCR4, CD 133, SCA-1, Tra-1-60, CD 44, CD 73, CD 90, CD 105 and Stro-1 can be used to stimulate an immune response against the stem cells expressing these markers.

In another preferred embodiment, stem cells isolated from a patient's tumor and expressing any one of Oct 3/4, Nanog, activated Stat-3, CXCR4, CD 133, SCA-1, Tra-1-60, CD 44, CD 73, CD 90, CD 105 and Stro-1, can be used to stimulate an immune response against these cells.

Vaccination can be conducted by conventional methods. For example, an immunogenic protein, such as any one of Oct 3/4, Nanog, activated Stat-3, CXCR4, CD 133, SCA-1, Tra-1-60, CD 44, CD 73, CD 90, CD 105 and Stro-1, can be used in a suitable diluent such as saline or water, or complete or incomplete adjuvants. Further, the immunogenic protein may or may not be bound to a carrier to make the protein more immunogenic. Examples of such carrier molecules include but are not limited to bovine serum albumin (BSA), keyhole limpet hemocyanin (KLH), tetanus toxoid, and the like. The immunogenic protein can be administered by any route appropriate for eliciting T cell response such as intravenous, intraperitoneal, intramuscular, subcutaneous, and the like. The immunogenic protein may be administered once or at periodic intervals until a T cell response is elicited. Doses of immunogenic protein effective to elicit a T cell response range from about 0.00001 to about 10 mg/kg. Doses of immunogenic protein-encoding expression vector effective to elicit a T cell response range from about 10⁵ to about 10⁷ plaque forming units. T cell response may be detected by a variety of methods known to those skilled in the art, including but not limited to, cytotoxicity assay, proliferation assay and cytokine release assays.

Vaccines made with the whole protein antigen are advantageous because they have the capability of stimulating an immune response against all of the potential antigenic sites expressed by the protein. Vaccines made with peptide antigens (e.g., 7-15 or 8-12 contiguous amino acids of the whole protein), on the other hand, will generally stimulate an immune response against fewer than all of the potential antigenic sites expressed by the protein. Peptide-based vaccines are sometimes advantageous over whole protein-based vaccines where it is desired to more specifically target the stimulated immune response, e.g., to avoid undesired cross reactions. For example, peptides for use in the vaccine can be selected to correspond to (1) specific epitopes of the antigens that are known to be presented by MHC class I or MHC class II molecules, or (2) a modified form of an epitope that either exhibits an increased stability in vivo or a higher binding affinity for an MHC molecule than the native epitope, while still being capable of specific activation of T-cells. See, Ayyoub et al., J. Biol. Chem., 274: 10227-10234, 1999; Parkhurst et al., Immunol., 157: 2539-2548, 1996. Peptide-based vaccines have been shown to circumvent immune tolerance to the intact proteins. Disis et al., J. Imunol., 156: 3151-3158, 1996.

In the methods of the present invention, an effective amount of the stem cell marker, for example Oct 3/4, or portion thereof joined to the molecule against which an immune response is desired is administered to an individual (e.g., mammal such as human). As used herein an “effective amount” is an amount that induces a CD4⁺ T cell independent immune response to the molecule in an individual. In a particular embodiment, an “effective amount” is an amount such that when administered to an individual, it results in an enhanced CD8⁺ CTL response to the molecule relative to the CD8⁺ CTL response to the molecule in an individual to whom an effective amount was not administered. For example, an effective amount or dosage of the Oct 3/4 or portion thereof is in the range of about 50 pmoles to about 5000 pmole. In one embodiment, the dosage range is from about 80 pmole to about 3500 pmoles; in another embodiment, the dosage range is from about 100 pmoles to about 2000 pmoles; and in a further embodiment the dosage range is from about 120 pmoles to about 1000 pmoles. The appropriate dosage of Oct 3/4 or portion thereof against which an immune response is desired for each individual will be determined by taking into consideration, for example, the particular Oct 3/4 and/or molecule being administered, the type of individual to whom the composition is being administered, the age and size of the individual, the condition or disease being treated or prevented and the severity of the condition or disease. Those skilled in the art will be able to determine using no more than routine experimentation the appropriate dosage to administer to an individual.

The stem cell marker, e.g. Oct 3/4 or portion thereof against which the immune response is desired can be administered to the individual in a variety of ways. The routes include intradermal, transdermal, (e.g., slow release polymers), intramuscular, intraperitoneal, intravenous, subcutaneous, oral, epidural and intranasal routes. Any other convenient route of administration can be used, for example, infusion or bolus injection, infusion of multiple injections over time, or absorption through epithelial or mucocutaneous linings. In addition, the Oct 3/4 can be administered with other components or biologically active agents, such as adjuvants, pharmaceutically acceptable surfactants (e.g., glycerides), excipients, (e.g., lactose), liposomes, carriers, diluents and vehicles.

Further, in the embodiment in which the molecule is a protein (peptide), the Oct 3/4 or portion thereof can be administered by in vivo expression of polynucleotides encoding such into an individual. For example, the Oct 3/4 or portion thereof and/or the molecule can be administered to an individual using a vector, wherein the vector which includes the Oct 3/4 or portion thereofjoined to the molecule is administered under conditions in which the Oct 3/4 or portion thereof and the molecule are expressed in vivo.

Several expression system vectors that can be used are available commercially or can be produced according to recombinant DNA and cell culture techniques. For example, vector systems such as yeast or vaccinia virus expression systems, or virus vectors can be used in the methods and compositions of the present invention (Kaufman, R. J., J. Meth. Cell and Mol. Biol, 2:221-236 (1990)). Other techniques using naked plasmids or DNA, and cloned genes encapsulated in targeted liopsomes or in erythrocyte ghosts can be used to introduce the Oct 3/4 or portion joined to the molecule into the host (Friedman, T., Science, 244:1275-1281 (1991); Rabinovich, N. R., et al., Science, 265:1410-1404 (1994)). The construction of expression vectors and the transfer of vectors and nucleic acids into various host cells can be accomplished using genetic engineering techniques, as described in manuals like Molecular Cloning and Current Protocols in Molecular Biology, which are incorporated by reference, or by using commercially available kits (Sambrook, J. et al., Molecular Cloning, Cold Spring Harbor Press, 1989; Ausubel, F. M., et al., Current Protocols in Molecular Biology, Greene Publishing Associates and Wiley-Interscience, 1989).

The proteins/peptides can also be administered per se or as an active ingredient in a pharmaceutical composition which may further include a pharmaceutically acceptable carrier. Preferably, one or more peptides are presented in context of an antigen presenting cell. The most common cells used to load antigens are bone marrow and peripheral blood derived dendritic cells (DC), as these cells express costimulatory molecules that help activation of CTL. Nevertheless, the peptide presenting cell can also be a macrophage, a B cell or a fibroblast. Presenting the peptide can be effected by a variety of methods, such as, but not limited to, (a) transforming the presenting cell with at least one polynucleotide (e.g., DNA) encoding at least one peptide; (b) loading the presenting cell with at least one polynucleotide (e.g., RNA) encoding at least one peptide; (c) loading the presenting cell with at least one peptide (e.g., synthetic peptide); and (d) loading the antigen presenting cell with at least one longer polypeptide (e.g., purified) including at least one peptide. Loading can be external or internal. The polynucleotide, peptide or longer polypeptide can be fused to internalizing sequences, antennapedia sequences or toxoid sequences or to helper sequences, such as, but not limited to, heat shock protein sequences.

For any peptide used in the methods of the invention, the therapeutically effective amount or dose can be estimated initially from activity assays in cell cultures and/or animals. For example, a dose can be formulated in animal models to achieve a circulating concentration range that includes the IC₅₀ as determined by activity assays (e.g., the concentration of the test compound, which achieves a half-maximal inhibition of the proliferation activity). Such information can be used to more accurately determine useful doses in humans.

Toxicity and therapeutic efficacy of the peptides described herein can be determined by standard pharmaceutical procedures in experimental animals, e.g., by determining the IC₅₀ and the LD₅₀ (lethal dose causing death in 50% of the tested animals) for a subject compound. The data obtained from these activity assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1). Dosage amount and interval may be adjusted individually to provide plasma levels of the active moiety which are sufficient to maintain therapeutic effects, termed the minimal effective concentration (MEC). The MEC will vary for each preparation, but can be estimated from in vitro and/or in vivo data, e.g., the concentration necessary to achieve 50-90% inhibition of a proliferation of certain cells may be ascertained using the assays described herein. Dosages necessary to achieve the MEC will depend on individual characteristics and route of administration. HPLC assays or bioassays can be used to determine plasma concentrations. Dosage intervals can also be determined using the MEC value. Preparations should be administered using a regimen, which maintains plasma levels above the MEC for 10-90% of the time, preferable between 30-90% and most preferably 50-90%. Depending on the severity and responsiveness of the condition to be treated, dosing can also be a single administration of a slow release composition described hereinabove, with course of treatment lasting from several days to several weeks or until cure is effected or diminution of the disease state is achieved. The amount of a composition to be administered will, of course, be dependent on the subject being treated, the severity of the affliction, the manner of administration, the judgment of the prescribing physician, etc.

The immune response can also be elicited using immune cells. Immune cells, such as T-cells, dendritic cells, macrophages, monocytes can be isolated from a patient. These cells can be sorted according to the desired immune cell based on cell lineage markers. For example, dendritic cell, T-cell etc. The cells are cultured either with peptides, such as Oct 3/4, Nanog, activated Stat-3, CXCR4, CD 133, SCA-1, Tra-1-60, CD 44, CD 73, CD 90, CD 105 and Stro-1, or the isolated stem cells. If antigen specific T cells are desired, T cells are cultured with the stem cells and/or above proteins or peptides, for about 7 to about 90 days (Yanelli, J. R. J Iminunol. Methods 139: 1-16 (1991)) and then screened to determine the clones of the desired reactivity against each or selected peptide or protein, for example Oct 3/4, using known methods of assaying T cell reactivity; T cells producing the desired reactivity are thus selected.

Alternatively, or in addition to directly stimulating an antigen specific immune cell (e.g. B and T lymphocytes), a patient's antigen presenting cells (e.g. dendritic cells, macrophages etc) can be isolated and cultured with the isolated stem cells or proteins such as such as Oct 3/4, Nanog, activated Stat-3, CXCR4, CD 133, SCA-1, Tra-1-60, CD 44, CD 73, CD 90, CD 105 and Stro-1. Upon re-infusion into a patient of the antigen presenting cells, these cells will present the proteins in various immunogenic forms to the antigen-specific cells, which in turn become activated and target stem cells expressing such as Oct 3/4, Nanog, activated Stat-3, CXCR4, CD 133, SCA-1, Tra-1-60, CD 44, CD 73, CD 90, CD 105 and Stro-1.

In a preferred embodiment, the antigen presenting cells are dendritic cells. The dendritic cells used in this invention can be isolated as described herein or by methods known to those skilled in the art. In a preferred embodiment, human dendritic cells are used from an appropriate tissue source, preferably blood or bone marrow.

Mature dendritic cells can also be obtained by culturing proliferating or non-proliferating dendritic cell precursors in a culture medium containing factors which promote maturation of immature dendritic cells to mature dendritic cells. Steinman et al. U.S. Pat. No. 5,851,756 and WO 97/29182 report methods and compositions for obtaining dendritic cells and are incorporated herein by reference.

The dendritic cell precursors, from which the immature dendritic cells are present in blood as PMBCs. Although most easily obtainable from blood, the precursor cells may also be obtained from any tissue. in which they reside, including bone marrow and spleen tissue. When cultured in the presence of cytokines such as a combination of GM-CSF and IL-4 or IL-13 as described below, the non-proliferating precursor cells give rise to immature dendritic cells for use in this invention.

Dendritic cell development can be divided into 4 stages: 1) a proliferating progenitor that can be either dendritic cell committed or uncommitted and capable of maturing to a nondendritic cell, 2) a non-proliferating precursor like the blood monocyte that does not show dendritic cell properties but is the starting population for many clinical studies, 3) an immature dendritic cell which has properties and commitment to become a dendritic cell, e.g. specialized antigen capture mechanisms including apoptotic cells for presentation, and MHC rich compartments, and 4) finally, the mature T cell stimulatory dendritic cell.

Cultures of immature dendritic cells, i.e. antigen-capturing phagocytic dendritic cells, may be obtained by culturing the non-proliferating precursor cells in the presence of cytokines which promote their differentiation. A combination of GM-CSF and IL-4 at a concentration of each at between about 200 to about 2000 U/ml, more preferably between about 500 and 1000 U/ml, and most preferably about 800 U/ml (GM-CSF) and 1000 U/ml (IL-4) produces significant quantities of the immature, i.e. antigen-capturing phagocytic dendritic cells, dendritic cells. Other cytokines or methods known in the art which efficiently generate immature dendritic cells may be used for purposes of this invention. Examples of other cytokines which promote differentiation of precursor cells into immature dendritic cells include, but are not limited to, IL-13. Maturation of dendritic cells requires the addition to the cell environment, preferably the culture medium, of a dendritic cell maturation factor which may be selected from monocyte conditioned medium and/or factors including TNF-α, IL-6, IFN-α, and IL-1-β. Alternatively, a mixture of stem cells isolated from a tumor, tumor cells and/or necrotic stem cell lysate may be added to induce maturation.

Apoptotic cells may be used to deliver antigen to either immature or mature dendritic cells, either freshly isolated or obtained from in vitro culture. In a preferred embodiment, stem cells isolated from a tumor comprising an antigen, such as Oct 3/4, Nanog, activated Stat-3, CXCR4, CD 133, SCA-1, Tra-1-60, CD 44, CD 73, CD 90, CD 105 and Stro-1, are co-cultured with immature dendritic cells for a time sufficient to allow the antigen to be internalized by the immature dendritic cells. These immature dendritic cells are then caused to mature by the addition of a maturation factor to the culture medium. The matured dendritic cells expressing processed antigen on their surface are then exposed to T cells for potent CTL induction.

In another embodiment, apoptotic stem cells may be used to deliver antigen to mature dendritic cells through surface receptors that enhance internalization of the apoptotic cells.

In another embodiment, apoptotic cells may be used to deliver antigen to immature dendritic cells that are maintained as immature dendritic cells as a means of efficiently modulating T cell tolerance or immunity in situ.

In a preferred embodiment, peripheral blood mononuclear cells (PBMCs) can be isolated from blood by sedimentation techniques. T cell-enriched (ER⁺) and T cell-depleted (ER—) populations can be prepared by resetting with neuraminidase treated sheep red blood cells. Dendritic cells are prepared from the ER⁻ cells and are preferably cultured for 7 days to 10 days in the presence of GM-CSF and IL-4. On about day 7 through 10, stem cells can be co-cultured with the dendritic cells and the dendritic cells caused to mature over the next four days with the addition of monocyte conditioned medium, a signal for maturation.

According to a preferred method of this invention, when bone marrow is used as the tissue source, B cells are removed prior to culturing of bone marrow in GM-CSF. While B cells and pre-B cells do not grow in response to GM-CSF, they represent approximately 50% of the initial marrow suspension and thereby preclude the use of staining with anti-Ia monoclonal antibodies to quickly enumerate dendritic cells. Additionally, granulocytes are GM-CSF responsive and readily proliferate in the presence of GM-CSF. As such, the B cells and granulocytes mask the presence of dendritic cell precursors. B cells can express the M342 and 2A1 granular antigens that are useful markers for distinguishing dendritic cells from macrophages and granulocytes. Moreover, granulocytes have a tendency to overgrow the cultures and compete for available GM-CSF. The most preferred method under this invention is to remove the majority of nonadherent, newly-formed granulocytes from the bone marrow cultures by gentle washes during the first 2-4 days in culture.

Preferably, in one form of pretreatment cells which compete and mask the proliferation of precursor dendritic cells are killed. Such pretreatment comprises killing cells expressing antigens which are not expressed on dendritic precursor cells by contacting bone marrow with antibodies specific for antigens not present on dendritic precursor cells in a medium comprising complement. Another form of pretreatment to remove undesirable cells suitable for use with this invention is adsorbing the undesirable precursor cells or their precursors onto a solid support using antibodies specific for antigens expressed on the undesirable cells. Several methods of adsorbing cells to solid supports of various types are known to those skilled in the art and are suitable for use with this invention. For example, undesirable cells may be removed by “panning” using a plastic surface such as a Petri dish. Alternatively, other methods which are among those suitable include adsorbing cells onto magnetic heads to be separated by a magnetic force; or immunobeads to be separated by gravity. Non adsorbed cells containing an increased proportion of dendritic cell precursors may then be separated from the cells adsorbed to the solid support by known means including panning. These pretreatment step serves a dual purpose: they destroy or revive the precursors of non-dendritic cells in the culture while increasing the proportion of dendritic cell precursors competing for GM-CSF in the culture.

In addition, Ia-positive cells, i.e. B cells and macrophages preferably are killed by culturing the cells in the presence of a mixture of anti Ia-antibodies, preferably monoclonal antibodies, and complement. Mature dendritic cells which are also present in bone marrow are also killed when the cells from the bone marrow are cultured in the presence of anti Ia-antibodies, however, these mature dendritic cells occur in such low quantities in the blood and bone marrow and possess such distinct antigenic markers from dendritic cell precursors that killing of these mature dendritic cells will not significantly effect the proliferation and yield of dendritic cell precursors. T and B cells as well as monocytes which also may be present in the bone marrow may be killed by including antibodies directed against T and B cell antigens and monocytes. Such antigens include but are not limited to CD3, CD4, the B cell antigen B220, thy-1, CD8 and monocyte antigens. The remaining viable cells from the bone marrow are then cultured in medium supplemented with about 500-1000 U/ml GM-CSF and cultured as described below. It should be noted that CD4 and CD8 antigens may be present on young dendritic cell precursors, therefore, antibodies directed to these antigens may deplete the dendritic cell precursor populations.

When blood is used as a tissue source, blood leukocytes may be obtained using conventional methods which maintain their viability. According to the preferred method of the invention, blood is diluted into medium (preferably RPMI) containing heparin (about 100 U/ml) or other suitable anticoagulant. The volume of blood to medium is about 1 to 1. Cells are pelleted and washed by centrifugation of the blood in medium at about 1000 rpm (150 g) at 4° C. Platelets and red blood cells are depleted by suspending the cell pellet in a mixture of medium and ammonium chloride. Preferably the mixture of medium to ammonium chloride (final concentration 0.839 percent) is about 1:1 by volume. Cells are pelleted by centrifugation and washed about 2 more times in the medium-ammonium chloride mixture, or until a population of leukocytes, substantially free of platelets and red blood cells, is obtained. Any isotonic solution commonly used in tissue culture may be used as the medium for separating blood leukocytes from platelets and red blood cells. Examples of such isotonic solutions are phosphate buffered saline, Hanks balanced salt solution, or complete growth mediums including for example RPMI 1640. RPMI 1640 is preferred.

Cells obtained from treatment of the tissue source are cultured to form a primary culture on an appropriate substrate in a culture medium supplemented with GM-CSF or a GM-CSF derivative protein or peptide having an amino acid sequence which sequence maintains biologic activity typical of GM-CSF. The appropriate substrate may be any tissue culture compatible surface to which cells may adhere. Preferably, the substrate is commercial plastic treated for use in tissue culture. Examples include various flasks, roller bottles, Petri dishes and multi-well containing plates made for use in tissue culture. Surfaces treated with a substance, for example collagen or poly-L-lysine, or antibodies specific for a particular cell type to promote cell adhesion may also be used provided they allow for the differential attachment of cells as described below. Cells are preferably plated at an initial cell density of about 7.5×10⁵ cells per cm².

When bone marrow which has been treated to reduce the proportion of non-dendritic cell precursors is cultured, aggregates comprising proliferating dendritic cell precursors are formed. The Ia-negative marrow nonlymphocytes comprising dendritic cell precursors are preferably cultured in high numbers, about 10⁶/well (5×10⁵ cells/cm²). Liquid marrow cultures which are set up for purposes other than culturing dendritic cell precursors are typically seeded at 1/10th this dose, but it is then difficult to identify and isolate the aggregates of developing dendritic cells.

The growth medium for the cells at each step of the method of the invention should allow for the survival and proliferation of the precursor dendritic cells. Any growth medium typically used to culture cells may be used according to the method of the invention provided the medium is supplemented with GM-CSF. Preferred medias include RPMI 1640, DMEM and A-MEM, with added amino acids and vitamins supplemented with an appropriate amount of serum or a defined set of hormones and an amount of GM-CSF sufficient to promote proliferation of dendritic precursor cells. Serum-free medium supplemented with hormones is also suitable for culturing the dendritic cell precursors. RPMI 1640 supplemented with 5% fetal calf serum (FCS) and GM-CSF is preferred. Cells may be selected or adapted to grow in other serums and at other concentrations of serum. Cells from human tissue may also be cultured in medium supplemented with human serum rather than FCS. Medias may contain antibiotics to minimize bacteria infection of the cultures. Penicillin, streptomycin or gentamicin or combinations containing them are preferred. The medium, or a portion of the medium, in which the cells are cultured should be periodically replenished to provide fresh nutrients including GM-CSF.

Besides monocyte conditioned medium, a combination of cytokines may be used to induce maturation of the immature dendritic cells. Examples of cytokines which may be used alone or in combination with each other include, but are not limited to, TNF-α, IL-1β, IL-6, IFN-α, pathogens, autoimmune antigens and necrotic cells.

If desired, apoptotic cell-activated dendritic cells made according to the method described above are used for induction of CTL responses. Delivery of antigen to mature dendritic cells, or alternatively, immature dendritic cells that are not caused to mature in vitro, is also within the scope of this invention.

Apoptotic cells, e.g. stem cells of the invention, useful for practicing the method of this invention should efficiently trigger antigen internalization by dendritic cells, and once internalized, facilitate translocation of the antigen to the appropriate antigen processing compartment.

In a preferred embodiment, the apoptotic cells, or fragments, blebs or bodies thereof, are internalized by the dendritic cells and targeted to an MHC class I processing compartment for activation of class I-restricted CD8⁺ cytotoxic T cells.

In another embodiment, the apoptotic cells can be used to activate class II-restricted CD4⁺ T helper cells by targeting antigen via the exogenous pathway and charging MHC class II molecules. Apoptotic cells, blebs and bodies are acquired by dendritic cells by phagocytosis. When a population of CD4⁺ cells is co-cultured with apoptotic cell-primed dendritic cells, the CD4⁺ T cells are activated by dendritic cells that have charged their MHC class II molecules with antigenic peptides. The apoptotic cell-charged dendritic cells of this invention activate antigen-specific CD4⁺ T cells with high efficiency.

For purposes of this invention, any cell type which expresses antigen and is capable of undergoing apoptosis can potentially serve as a donor cell for antigen delivery to the potent dendritic cell system. Examples of antigen that can be delivered to dendritic cells by donor cells include, but are not limited to, viral, bacterial, protozoan, microbial and tumor antigens as well as self-antigens. Preferred antigens for priming dendritic cells in vitro or in vivo are derived from the stem cells described herein. Additional antigens include tumor antigens derived from the tumors.

The population of donor cells expressing antigen can be induced to undergo apoptosis in vitro or in vivo using a variety of methods known in the art including, but not limited to, viral infection, irradiation with ultraviolet light, gamma radiation steroids, cytokines or by depriving donor cells of nutrient's in the cell culture medium. Time course studies can establish incubation periods sufficient for optimal induction of apoptosis in a population of donor cells. For example, monocytes infected with influenza virus begin to express early markers for apoptosis by 6 hours after infection. Examples of specific markers for apoptosis include Annexin V, TUNEL⁺ cells, DNA laddering and uptake of propidium iodide. Those skilled in the art will recognize that optimal timing for apoptosis will vary depending on the donor cells and the technique employed for inducing apoptosis. Cell death can be assayed by a variety of methods known in the art including, but not limited to, fluorescence staining of early markers for apoptosis, and determination of percent apoptotic cells by standard cell sorting techniques.

Once donor cells expressing either native or foreign antigen have been induced to undergo apoptosis they can be contacted with an appropriate number of dendritic cells in vitro or in vivo. For most antigens a ratio of only about 1-10 donor cells to 100 immature dendritic cells is suitable for priming the dendritic cells. Higher numbers of cells are preferred if mature dendritic cells are to be primed, preferably 100 to 1000 donor cells to mature dendritic cells. Efficiency of cross-priming or cross-tolerizing dendritic cells can be determined by assaying T cell cytolytic activity in vitro or using dendritic cells as targets of CTLs. Other methods known to those skilled in the art may be used to detect the presence of antigen on the dendritic cell surface following their exposure to the stem donor cells. Moreover, those skilled in the art will recognize that the length of time necessary for an antigen presenting cell to phagocytose apoptotic cells, or cell fragments, may vary depending on the cell types and antigens used.

An important feature of the dendritic cells is the capacity to efficiently present antigens on both MHC class I and class II molecules. Antigens are acquired by dendritic cells through the exogenous pathway by phagocytosis and as a result also efficiently charge MHC II molecules. CD4⁺ T cells may be activated by the dendritic cells presenting antigenic peptide which is complexed with MHC II using the method according to this invention, since it is known in the art that dendritic cells are the most potent inducers of CD4⁺ helper T cell immunity. CD4⁺ T cells can provide critical sources of help, both for generating active CD8⁺ and other killer T cells during the acute response to antigen, and for generating the memory that is required for long term resistance and vaccination. Thus, by using apoptotic cells to charge MHC class I and/or II products, efficient T cell modulation in situ can be achieved. Preferably immature dendritic cells are contacted with the donor cells and both class I and II MHC receptors become optimally charged with antigen. In addition, the dendritic cells are matured by the presence of the necrotic cells which also contributes to MHC class II loading. An important advantage of priming dendritic cells with desired antigen is that even poorly defined or undefined antigens can be routed to the appropriate antigen processing compartment of the dendritic cells to generate antigen-specific T cell responses. Moreover, dendritic cells can be charged with multiple antigens on multiple MHCs to yield poly- or oligoclonal stimulation of T cells. Thus, since the starting material can be obtained from virtually any tissue.

Accordingly, antigen can be provided to the dendritic cells by proteins, phospholipids, carbohydrates and/or glycolipids, which enhance internalization and translocation of antigen to the appropriate antigen processing compartment in the dendritic cells. Thus, it is contemplated that liposomes comprising antigen may enhance delivery of antigen to dendritic cells. Liposomes enhanced with antigens comprising Oct 3/4, Nanog, activated Stat-3, CXCR4, CD 133, SCA-1, Tra-1-60, CD 44, CD 73, CD 90, CD 105 and Stro-1 can also be used for delivering antigen in vivo to dendritic cells.

Phagocytoses of particulate matter by dendritic cell precursors may be accomplished by culturing the dendritic cell precursors in the presence of particulate matter for a time sufficient to allow the cells to phagocytose, process and present the antigen. Preferably, culturing of the cells in the presence of the particles should be for a period of between 1 to 48 hours. More preferably, culturing cells in the presence of particulate matter will be for about 20 hours. Those of skill in the art will recognize that the length of time necessary for a cell to phagocytose a particle will be dependent on the cell type and the nature of the particle being phagocytosed. Methods to monitor the extent of such phagocytosis are well known to those skilled in the art.

Cells should be exposed to antigen for sufficient time to allow antigens to be internalized and presented on the cell surface. The time necessary for the cells to internalize and present the processed antigen may be determined using pulse-chase protocols in which exposure to antigen is followed by a wash-out period. Once the minimum time necessary for cells to express processed antigen on their surface is determined, a pulse-chase protocol may be used to prepare cells and antigens for eliciting immunogenic responses.

The phagocytic dendritic precursor cells are obtained by stimulating cell cultures comprising dendritic precursor cells with GM-CSF to induce aggregates of growing dendritic cells. These dendritic precursor cells may be obtained from any of the source tissues containing dendritic cell precursors described above. Preferably, the source tissue is bone marrow or blood cultures. Cells within these aggregates are clearly phagocytic. If the developing cultures are exposed to particles, washed and “chased” for 2 days, the number of MHC-class II rich dendritic cells increases substantially.

The antigen charged dendritic cells or the antigen-specific T lymphocytes generated by these dendritic cells may be used for either a prophylactic or therapeutic purpose. For activating T cells in an individual between about 2×10⁵ and 2×10⁹ more preferably between 1 million and 10 million apoptotic cell-activated mature dendritic cells should be administered to an individual. The dendritic cells should be administered in a physiologically compatible carrier which is nontoxic to the cells and the individual. Such a carrier may be the growth medium described above, or any suitable buffering medium such as phosphate buffered saline (PBS). The mature dendritic cells prepared according to this invention are particularly potent at activating T cells. For activating T cells in vitro the ratio of dendritic cells to T cells is between 1:10 and 1:1000. More preferably, between 1:30 and 1:150. Between approximately 10⁶ and 10⁹ or more activated T cells are administered back to the individual to produce a response against the antigen.

Dendritic cells may be administered to an individual using standard methods including intravenous, intraperitoneal, subcutaneously, intradermally or intramuscularly. The homing ability of the dendritic cells facilitates their ability to find T cells and cause their activation. To use antigen-activated dendritic cells as a therapeutic or immunogen the antigen-activated dendritic cells are injected by any method which elicits an immune response into a syngeneic animal or human. Preferably, dendritic cells are injected back into the same animal or human from whom the source tissue was obtained. The number of antigen-activated dendritic cells reinjected back into the animal or human in need of treatment may vary depending on inter alia, the antigen and size of the individual. A key feature in the function of dendritic cells in situ is the capacity to migrate or home to the T-dependent regions of lymphoid tissues, where the dendritic cells would be in an optimal position to select the requisite antigen-reactive T cells from the pool of recirculating quiescent lymphocytes and thereby initiate the T-dependent response.

According to the preferred method of stimulating an immune response in an individual, a tissue source from that individual would be identified to provide the dendritic cell precursors. If blood is used as the tissue source preferably the individual is first treated with cytokine to stimulate hematopoieses. After isolation and expansion of the dendritic cell precursor population, the cells are contacted with the antigen. Preferably, contact with the antigen is conducted in vitro, as described infra. After sufficient time has elapsed to allow the cells to process and present the antigen on their surfaces, the cell-antigen complexes are put back into the individual in sufficient quantity to evoke an immune response. Preferably between 1×10⁶ and 1×10⁷ antigen presenting cells are injected back into the individual.

If desired, the antigens can be modified. Modified antigens are prepared by combining substances to be modified or other antigens with the dendritic cells prepared according to the method described herein. The dendritic cells process or modify antigens in a manner which promotes the stimulation of T-cells by the processed or modified antigens. Such dendritic cell modified antigens are advantageous because they can be more specific and have fewer undesirable epitopes than non-modified T-dependent antigens. The dendritic cell modified antigens may be purified by standard biochemical methods. For example, it is known to use antibodies to products of the major histocompatibility complex (MHC) to select MHC-antigenic peptide complexes and then to elute the requisite processed peptides with acid [Rudensky et al., Nature 353:622-7 (1991); Hunt et al., Science 255: 1261-3 (1992)] which are incorporated herein by reference.

Antigen-activated dendritic cells and dendritic cell modified antigens may both be used to elicit an immune response against an antigen. The activated dendritic cells or modified antigens may by used as vaccines to prevent future infection or may be used to activate the immune system to treat ongoing disease. The activated dendritic cells or modified antigens may be formulated for use as vaccines or pharmaceutical compositions with suitable carriers such as physiological saline or other injectable liquids. The vaccines or pharmaceutical compositions comprising the modified antigens or the antigen-activated dendritic cells would be administered in therapeutically effective amounts sufficient to elicit an immune response. Preferably, between about 1 to 100 micrograms of modified antigen, or its equivalent when bound to dendritic cells, should be administered per dose.

To determine antigen specific T cells, methods for assessing the cytotoxic activity of T lymphocytes, and in particular the ability of cytotoxic T lymphocytes to be induced by antigen presenting dendritic cells include a sample comprising T lymphocytes to be assayed for cytotoxic activity. Preferably, the cells are obtained from an individual from whom it is desirable to assess their capacity to provoke a cytotoxic T lymphocyte response. The T lymphocytes are then exposed to antigen presenting dendritic cells which have been caused to present antigen. Preferably, the dendritic cells have been primed with cells which express antigen. After an appropriate period of time, which may be determined by assessing the cytotoxic activity of a control population of T lymphocytes which are known to be capable of being induced to become cytotoxic cells, the T lymphocytes to be assessed are tested for cytotoxic activity in a standard cytotoxic assay. Such assays may include, but are not limited to, a chromium release assay.

### Production of Antibodies

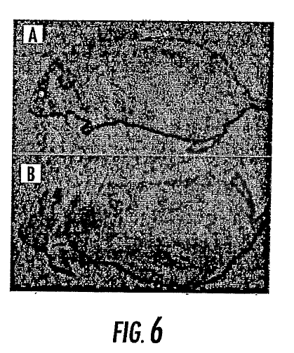

Tumor biomarkers obtained from samples in patients suffering from tumors and the like, can be prepared from the isolated tumors from sarcospheres established from a human sarcoma biopsy and from a canine osteosarcoma cell line, as described in detail in the examples which follow. (see, also FIG. 6A-6B). The isolated tumor stem cells can be used whole to generate antibodies specific for the markers or markers can be isolated, for example, Oct 3/4, Nanog, activated Stat-3, CXCR4, CD 133, SCA-1, Tra-1-60, CD 44, CD 73, CD 90, CD 105 and Stro-1.

Furthermore, tumor biomarkers, e.g. Oct 3/4, Nanog, activated Stat-3, CXCR4, CD 133, SCA-1, Tra-1-60, CD 44, CD 73, CD 90, CD 105 and Stro-1 can be subjected to enzymatic digestion to obtain fragments or peptides of the biomarkers for the production of antibodies to different antigenic epitopes that can be present in a peptide versus the whole protein. Antigenic epitopes are useful, for example, to raise antibodies, including monoclonal antibodies, that specifically bind the epitope. Antigenic epitopes can be used as the target molecules in immunoassays. (See, for instance, Wilson et al., Cell 37:767-778 (1984); Sutcliffe et al., Science 219:660-666 (1983)).

Tumor biomarker epitopes can be used, for example, to induce antibodies according to methods well known in the art. (See, for instance, Sutcliffe et al., supra; Wilson et al., supra; Chow et al., Proc. Natl. Acad. Sci. USA 82:910-914; and Bittle et al., J. Gen. Virol. 66:2347-2354 (1985). Tumor polypeptides comprising one or more immunogenic epitopes may be presented for eliciting an antibody response together with a carrier protein, such as an albumin, to an animal system (such as rabbit or mouse), or, if the polypeptide is of sufficient length (at least about 25 amino acids), the polypeptide may be presented without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting).

Epitope-bearing polypeptides of the present invention may be used to induce antibodies according to methods well known in the art including, but not limited to, in vivo immunization, in vitro immunization, and phage display methods. See, e.g., Sutcliffe et al., supra; Wilson et al., supra, and Bittle et al., J. Gen. Virol., 66:2347-2354 (1985). If in vivo immunization is used, animals may be immunized with free peptide; however, anti-peptide antibody titer may be boosted by coupling the peptide to a macromolecular carrier, such as keyhole limpet hemacyanin (KLH) or tetanus toxoid. For instance, peptides containing cysteine residues may be coupled to a carrier using a linker such as maleimidobenzoyl-N-hydroxysuccinimide ester (MBS), while other peptides may be coupled to carriers using a more general linking agent such as glutaraldehyde. Animals such as rabbits, rats and mice are immunized with either free or carrier-coupled peptides, for instance, by intraperitoneal and/or intradermal injection of emulsions containing about 100 μg of peptide or carrier protein and Freund's adjuvant or any other adjuvant known for stimulating an immune response. Several booster injections may be needed, for instance, at intervals of about two weeks, to provide a useful titer of anti-peptide antibody which can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

Nucleic acid tumor biomarker epitopes can also be recombined with a gene of interest as an epitope tag (e.g., the hemaglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with the recombinant vaccinia virus are loaded onto Ni²⁺ nitriloacetic acid-agarose column and histidine-tagged proteins can be selectively eluted with imidazole-containing buffers.

The antibodies of the present invention may be generated by any suitable method known in the art. The antibodies of the present invention can comprise polyclonal antibodies. Methods of preparing polyclonal antibodies are known to the skilled artisan (Harlow, et al., Antibodies: A Laboratory Manual, (Cold spring Harbor Laboratory Press, 2nd ed. (1988), which is hereby incorporated herein by reference). For example, a polypeptide of the invention can be administered to various host animals including, but not limited to, rabbits, mice, rats, etc. to induce the production of sera containing polyclonal antibodies specific for the antigen. The administration of the polypeptides of the present invention may entail one or more injections of an immunizing agent and, if desired, an adjuvant. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guein) and Corynebacterium parvum. Such adjuvants are also well known in the art. For the purposes of the invention, “immunizing agent” may be defined as a polypeptide of the invention, including fragments, variants, and/or derivatives thereof, in addition to fusions with heterologous polypeptides and other forms of the polypeptides as may be described herein.

Typically, the immunizing agent and/or adjuvant will be injected in the mammal by multiple subcutaneous or intraperitoneal injections, though they may also be given intramuscularly, and/or through I.V. The immunizing agent may include polypeptides of the present invention or a fusion protein or variants thereof. Depending upon the nature of the polypeptides (i.e., percent hydrophobicity, percent hydrophilicity, stability, net charge, isoelectric point etc.), it may be useful to conjugate the immunizing agent to a protein known to be immunogenic in the mammal being immunized. Such conjugation includes either chemical conjugation by derivatizing active chemical functional groups to both the polypeptide of the present invention and the immunogenic protein such that a covalent bond is formed, or through fusion-protein based methodology, or other methods known to the skilled artisan. Examples of such immunogenic proteins include, but are not limited to keyhole limpet hemocyanin, serum albumin, bovine thyroglobulin, and soybean trypsin inhibitor. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Additional examples of adjuvants which may be employed includes the MPL-TDM adjuvant (monophosphoryl lipid A, synthetic trehalose dicorynomycolate). The immunization protocol may be selected by one skilled in the art without undue experimentation.

The antibodies of the present invention can also comprise monoclonal antibodies. Monoclonal antibodies may be prepared using hybridoma methods, such as those described by Kohler and Milstein, Nature, 256:495 (1975) and U.S. Pat. No. 4,376,110, by Harlow, et al., Antibodies: A Laboratory Manual, (Cold spring Harbor Laboratory Press, 2nd ed. (1988), by Hammerling, et al., Monoclonal Antibodies and T-Cell Hybridomas (Elsevier, N.Y., (1981)), or other methods known to the artisan. Other examples of methods which may be employed for producing monoclonal antibodies includes, but are not limited to, the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In a hybridoma method, a mouse, a humanized mouse, a mouse with a human immune system, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include tumor polypeptides, fragments or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986), pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. As inferred throughout the specification, human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the tumor polypeptides of the present invention. Preferably, the binding specificity of monoclonal antibodies produced by the hybridoma cells is determined by immunoprecipitation or by an in vitro binding assay, such as radioimmunoassay (RIA) or enzyme-linked immunoadsorbant assay (ELISA). Such techniques are known in the art and within the skill of the artisan. The binding affinity of the monoclonal antibody can, for example, be determined by the Scatchard analysis of Munson and Pollart, Anal. Biochem., 107:220 (1980).

After the desired hybridoma cells are identified, the clones may be subcloned by limiting dilution procedures and grown by standard methods (Goding, supra). Suitable culture media for this purpose include, for example, Dulbecco's Modified Eagle's Medium and RPMI-1640. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-sepharose, hydroxyapatite chromatography, gel exclusion chromatography, gel electrophoresis, dialysis, or affinity chromatography.

The skilled artisan would acknowledge that a variety of methods exist in the art for the production of monoclonal antibodies and thus, the invention is not limited to their sole production in hybridomas. For example, the monoclonal antibodies may be made by recombinant DNA methods, such as those described in U.S. Pat. No. 4,816,567. In this context, the term “monoclonal antibody” refers to an antibody derived from a single eukaryotic, phage, or prokaryotic clone. The DNA encoding the monoclonal antibodies of the invention can be readily isolated and sequenced using conventional procedures (e.g., by using oligonucleotide probes that are capable of binding specifically to genes encoding the heavy and light chains of murine antibodies, or such chains from human, humanized, or other sources). The hybridoma cells of the invention serve as a preferred source of such DNA. Once isolated, the DNA may be placed into expression vectors, which are then transformed into host cells such as Simian COS cells, Chinese hamster ovary (CHO) cells, or myeloma cells that do not otherwise produce immunoglobulin protein, to obtain the synthesis of monoclonal antibodies in the recombinant host cells.

Methods for producing and screening for specific antibodies using hybridoma technology are routine and well known in the art. In a non-limiting example, mice can be immunized with a biomarker polypeptide or a cell expressing such peptide. Once an immune response is detected, e.g., antibodies specific for the antigen are detected in the mouse serum, the mouse spleen is harvested and splenocytes isolated. The splenocytes are then fused by well-known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a polypeptide of the invention. The antibodies detecting tumor biomarkers, peptides and derivatives thereof, can be used in immunoassays and other methods to identify new tumor biomarkers and for use in the diagnosis of tumor, degree of severity of injury and/or neurological disorders.

Other methods can also be used for the large scale production of tumor biomarker specific antibodies. For example, antibodies can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference.

The antibodies of the present invention have various utilities. For example, such antibodies may be used in diagnostic assays to detect the presence or quantification of the polypeptides of the invention in a sample. Such a diagnostic assay can comprise at least two steps. The first, subjecting a sample with the antibody, wherein the sample is a tissue (e.g., human, animal, etc.), biological fluid (e.g., blood, urine, sputum, semen, amniotic fluid, saliva, etc.), biological extract (e.g., tissue or cellular homogenate, etc.), a protein microchip (e.g., See Arenkov P, et al., Anal Biochem., 278(2):123-131 (2000)), or a chromatography column, etc. And a second step involving the quantification of antibody bound to the substrate. Alternatively, the method may additionally involve a first step of attaching the antibody, either covalently, electrostatically, or reversibly, to a solid support, and a second step of subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), pp 147-158). The antibodies used in the diagnostic assays can be labeled with a detectable moiety. The detectable moiety should be capable of producing, either directly or indirectly, a detectable signal. For example, the detectable moiety may be a radioisotope, such as ²H, ¹⁴C, ³²P, or ¹²⁵I, a florescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et al., Nature, 144:945 (1962); David et al., Biochem., 13:1014 (1974); Pain et al., J. Immunol. Methods, 40:219 (1981); and Nygren, J. Histochem. Cytochem., 30:407 (1982).

### Isolation of Markers

In a preferred embodiment, a sample is obtained from the isolated tumors, sarcospheres, which includes whole cells, tissues etc.

In another preferred embodiment, a biological sample is obtained from a patient with a tumor. Biological samples comprising biomarkers from other patients and control subjects (i.e. normal healthy individuals of similar age, sex, physical condition) are used as comparisons. Preferably, the sample is prepared prior to detection of biomarkers.

Preferably, tumor cells are from any organ, tissue etc in the body. In addition, cellular damage can compromise the cell membrane leading to the efflux of these tumor proteins first into the extracellular fluid or space and to the biofluids (e.g. cerebrospinal fluid, urine, sweat, saliva, etc.) and eventually in the circulating blood. Thus, other suitable biological samples include, but not limited to such cells or fluid secreted from these cells. Obtaining biological fluids such as cerebrospinal fluid, blood, plasma, serum, saliva and urine, from a subject is typically much less invasive and traumatizing than obtaining a solid tissue biopsy sample. Thus, samples, which are biological fluids, are preferred for use in the invention. A biological sample can be obtained from a subject by conventional techniques. For example, CSF can be obtained by lumbar puncture. Blood can be obtained by venipuncture, while plasma and serum can be obtained by fractionating whole blood according to known methods. Surgical techniques for obtaining solid tissue samples are well known in the art. For example, methods for obtaining a nervous system tissue sample are described in standard neuro-surgery texts such as Atlas of Neurosurgery: Basic Approaches to Cranial and Vascular Procedures, by F. Meyer, Churchill Livingstone, 1999; Stereotactic and Image Directed Surgery of Brain Tumors, 1st ed., by David G. T. Thomas, WB Saunders Co., 1993; and Cranial Microsurgery: Approaches and Techniques, by L. N. Sekhar and E. De Oliveira, 1st ed., Thieme Medical Publishing, 1999. Methods for obtaining and analyzing brain tissue are also described in Belay et al., Arch. Neurol. 58: 1673-1678 (2001); and Seijo et al., J Clin. Microbiol. 38: 3892-3895 (2000).

Any animal can be used as a subject from which a biological sample is obtained, Preferably, the subject is a mammal, such as for example, a human, dog, cat, horse, cow, pig, sheep, goat, chicken, primate, rat, or mouse. More preferably, the subject is a human. Particularly preferred are cancer patients.

The biomarkers of the invention, e.g. Oct 3/4, Nanog, activated Stat-3, CXCR4, CD 133, SCA-1, Tra-1-60, CD 44, CD 73, CD 90, CD 105 and Stro-1, can be detected in a sample by any means. Other preferred molecules include, but not limited to: tenascins, proteoglycans, glycoproteins, glycolipids and other glycoconjugates that make up morphogenetic molecules and extracellular matrix molecules and their receptors, undulins and the like. Other non-limiting examples include polypeptide growth factors (e.g., FGFs1-9, PDGF, HGF, VEGF, TGF-β, IL-3); extracellular matrix components (e.g., laminins, fibronectins; thrombospondins, tenascins, collagens, VonWillebrand's factor); proteases and anti-proteases (e.g., thrombin, TPA, UPA, clotting factors Iand X, PAI-1); cell-adhesion molecules (e.g., N-CAM, LI, myelin-associated glycoprotein); proteins involved in lipoprotein metabolism (e.g., APO-B, APO-E, lipoprotein lipase); cell-cell adhesion molecules (e.g., N-CAM, myelin-associated glycoprotein, selecting, pecam); angiogenin; lactoferrin; viral proteins (e.g., proteins from HIV, herpes complex) and other compounds which bind to GAG.

Methods for detecting the biomarkers are known in the art. For example, immunoassays, include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, fluorescent immunoassays and the like. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding an antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody to immunoprecipitate a particular antigen can be assessed by, e.g., Western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen (i.e. tumor biomarker), coating the well of a 96 well microtiter plate with the antigen, adding the antibody of interest, e.g. antibodies to Oct 3/4, Nanog, activated Stat-3, CXCR4, CD 133, SCA-1, Tra-1-60, CD 44, CD 73, CD 90, CD 105 and Stro-1 and ligands thereof, conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

Typically, preparation involves fractionation of the sample and collection of fractions determined to contain the biomarkers. Methods of pre-fractionation include, for example, size exclusion chromatography, ion exchange chromatography, heparin chromatography, affinity chromatography, sequential extraction, gel electrophoresis and liquid chromatography. The analytes also may be modified prior to detection. These methods are useful to simplify the sample for further analysis. For example, it can be useful to remove high abundance proteins, such as albumin, from blood before analysis.

In one embodiment, a sample can be pre-fractionated according to size of proteins in a sample using size exclusion chromatography. For a biological sample wherein the amount of sample available is small, preferably a size selection spin column is used. In general, the first fraction that is eluted from the column (“fraction 1”) has the highest percentage of high molecular weight proteins; fraction 2 has a lower percentage of high molecular weight proteins; fraction 3 has even a lower percentage of high molecular weight proteins; fraction 4 has the lowest amount of large proteins; and so on. Each fraction can then be analyzed by immunoassays, gas phase ion spectrometry, and the like, for the detection of markers.

In another embodiment, a sample can be pre-fractionated by anion exchange chromatography. Anion exchange chromatography allows pre-fractionation of the proteins in a sample roughly according to their charge characteristics. For example, a Q anion-exchange resin can be used (e.g., Q HyperD F, Biosepra), and a sample can be sequentially eluted with eluants having different pH's. Anion exchange chromatography allows separation of biomarkers in a sample that are more negatively charged from other types of biomarkers. Proteins that are eluted with an eluant having a high pH is likely to be weakly negatively charged, and a fraction that is eluted with an eluant having a low pH is likely to be strongly negatively charged. Thus, in addition to reducing complexity of a sample, anion exchange chromatography separates proteins according to their binding characteristics.

In yet another embodiment, a sample can be pre-fractionated by heparin chromatography. Heparin chromatography allows pre-fractionation of the markers in a sample also on the basis of affinity interaction with heparin and charge characteristics. Heparin, a sulfated mucopolysaccharide, will bind markers with positively charged moieties and a sample can be sequentially eluted with eluants having different pH's or salt concentrations. Markers eluted with an eluant having a low pH are more likely to be weakly positively charged. Markers eluted with an eluant having a high pH are more likely to be strongly positively charged. Thus, heparin chromatography also reduces the complexity of a sample and separates markers according to their binding characteristics.

In yet another embodiment, a sample can be pre-fractionated by isolating proteins that have a specific characteristic, e.g. are glycosylated. For example, a CSF sample can be fractionated by passing the sample over a lectin chromatography column (which has a high affinity for sugars). Glycosylated proteins will bind to the lectin column and non-glycosylated proteins will pass through the flow through. Glycosylated proteins are then eluted from the lectin column with an eluant containing a sugar, e.g., N-acetyl-glucosamine and are available for further analysis.

Thus there are many ways to reduce the complexity of a sample based on the binding properties of the proteins in the sample, or the characteristics of the proteins in the sample.

In yet another embodiment, a sample can be fractionated using a sequential extraction protocol. In sequential extraction, a sample is exposed to a series of adsorbents to extract different types of biomarkers from a sample. For example, a sample is applied to a first adsorbent to extract certain proteins, and an eluant containing non-adsorbent proteins (i.e., proteins that did not bind to the first adsorbent) is collected. Then, the fraction is exposed to a second adsorbent. This further extracts various proteins from the fraction. This second fraction is then exposed to a third adsorbent, and so on.

Any suitable materials and methods can be used to perform sequential extraction of a sample. For example, a series of spin columns comprising different adsorbents can be used. In another example, a multi-well comprising different adsorbents at its bottom can be used. In another example, sequential extraction can be performed on a probe adapted for use in a gas phase ion spectrometer, wherein the probe surface comprises adsorbents for binding biomarkers. In this embodiment, the sample is applied to a first adsorbent on the probe, which is subsequently washed with an eluant. Markers that do not bind to the first adsorbent are removed with an eluant. The markers that are in the fraction can be applied to a second adsorbent on the probe, and so forth. The advantage of performing sequential extraction on a gas phase ion spectrometer probe is that markers that bind to various adsorbents at every stage of the sequential extraction protocol can be analyzed directly using a gas phase ion spectrometer.

In yet another embodiment, biomarkers in a sample can be separated by high-resolution electrophoresis, e.g., one or two-dimensional gel electrophoresis. A fraction containing a marker can be isolated and further analyzed by gas phase ion spectrometry. Preferably, two-dimensional gel electrophoresis is used to generate two-dimensional array of spots of biomarkers, including one or more markers. See, e.g., Jungblut and Thiede, Mass Spectr. Rev. 16:145-162 (1997).

The two-dimensional gel electrophoresis can be performed using methods known in the art. See, e.g., Deutscher ed., Methods In Enzymology vol. 182. Typically, biomarkers in a sample are separated by, e.g., isoelectric focusing, during which biomarkers in a sample are separated in a pH gradient until they reach a spot where their net charge is zero (i.e., isoelectric point). This first separation step results in one-dimensional array of biomarkers. The biomarkers in one dimensional array is further separated using a technique generally distinct from that used in the first separation step. For example, in the second dimension, biomarkers separated by isoelectric focusing are further separated using a polyacrylamide gel, such as polyacrylamide gel electrophoresis in the presence of sodium dodecyl sulfate (SDS-PAGE). SDS-PAGE gel allows further separation based on molecular mass of biomarkers. Typically, two-dimensional gel electrophoresis can separate chemically different biomarkers in the molecular mass range from 1000-200,000 Da within complex mixtures.

Biomarkers in the two-dimensional array can be detected using any suitable methods known in the art. For example, biomarkers in a gel can be labeled or stained (e.g., Coomassie Blue or silver staining). If gel electrophoresis generates spots that correspond to the molecular weight of one or more markers of the invention, the spot can be further analyzed by densitometric analysis or gas phase ion spectrometry. For example, spots can be excised from the gel and analyzed by gas phase ion spectrometry. Alternatively, the gel containing biomarkers can be transferred to an inert membrane by applying an electric field. Then a spot on the membrane that approximately corresponds to the molecular weight of a marker can be analyzed by gas phase ion spectrometry. In gas phase ion spectrometry, the spots can be analyzed using any suitable techniques, such as MALDI or SELDI.

Prior to gas phase ion spectrometry analysis, it may be desirable to cleave biomarkers in the spot into smaller fragments using cleaving reagents, such as proteases (e.g., trypsin). The digestion of biomarkers into small fragments provides a mass fingerprint of the biomarkers in the spot, which can be used to determine the identity of markers if desired.

In yet another embodiment, high performance liquid chromatography (HPLC) can be used to separate a mixture of biomarkers in a sample based on their different physical properties, such as polarity, charge and size. HPLC instruments typically consist of a reservoir of mobile phase, a pump, an injector, a separation column, and a detector. Biomarkers in a sample are separated by injecting an aliquot of the sample onto the column. Different biomarkers in the mixture pass through the column at different rates due to differences in their partitioning behavior between the mobile liquid phase and the stationary phase. A fraction that corresponds to the molecular weight and/or physical properties of one or more markers can be collected. The fraction can then be analyzed by gas phase ion spectrometry to detect markers.

Optionally, a marker can be modified before analysis to improve its resolution or to determine its identity. For example, the markers may be subject to proteolytic digestion before analysis. Any protease can be used. Proteases, such as trypsin, that are likely to cleave the markers into a discrete number of fragments are particularly useful. The fragments that result from digestion function as a fingerprint for the markers, thereby enabling their detection indirectly. This is particularly useful where there are markers with similar molecular masses that might be confused for the marker in question. Also, proteolytic fragmentation is useful for high molecular weight markers because smaller markers are more easily resolved by mass spectrometry. In another example, biomarkers can be modified to improve detection resolution. For instance, neuraminidase can be used to remove terminal sialic acid residues from glycoproteins to improve binding to an anionic adsorbent and to improve detection resolution. In another example, the markers can be modified by the attachment of a tag of particular molecular weight that specifically bind to molecular markers, further distinguishing them. Optionally, after detecting such modified markers, the identity of the markers can be further determined by matching the physical and chemical characteristics of the modified markers in a protein database (e.g., SwissProt).

After preparation, biomarkers in a sample are typically captured on a substrate for detection. Traditional substrates include antibody-coated 96-well plates or nitrocellulose membranes that are subsequently probed for the presence of proteins. Preferably, the biomarkers are identified using immunoassays as described above. However, preferred methods also include the use of biochips. Preferably the biochips are protein biochips for capture and detection of proteins. Many protein biochips are described in the art. These include, for example, protein biochips produced by Packard BioScience Company (Meriden Conn.), Zyomyx (Hayward, Calif.) and Phylos (Lexington, Mass.). In general, protein biochips comprise a substrate having a surface. A capture reagent or adsorbent is attached to the surface of the substrate. Frequently, the surface comprises a plurality of addressable locations, each of which location has the capture reagent bound there. The capture reagent can be a biological molecule, such as a polypeptide or a nucleic acid, which captures other biomarkers in a specific manner. Alternatively, the capture reagent can be a chromatographic material, such as an anion exchange material or a hydrophilic material. Examples of such protein biochips are described in the following patents or patent applications: U.S. Pat. No. 6,225,047 (Hutchens and Yip, “Use of retentate chromatography to generate difference maps,” May 1, 2001), International publication WO 99/51773 (Kuimelis and Wagner, “Addressable protein arrays,” Oct. 14, 1999), International publication WO 00/04389 (Wagner et al., “Arrays of protein-capture agents and methods of use thereof,” Jul. 27, 2000), International publication WO 00/56934 (Englert et al., “Continuous porous matrix arrays,” Sep. 28, 2000).

In general, a sample containing the biomarkers is placed on the active surface of a biochip for a sufficient time to allow binding. Then, unbound molecules are washed from the surface using a suitable eluant. In general, the more stringent the eluant, the more tightly the proteins must be bound to be retained after the wash. The retained protein biomarkers now can be detected by appropriate means.

Analytes captured on the surface of a protein biochip can be detected by any method known in the art. This includes, for example, mass spectrometry, fluorescence, surface plasmon resonance, ellipsometry and atomic force microscopy. Mass spectrometry, and particularly SELDI mass spectrometry, is a particularly useful method for detection of the biomarkers of this invention.

Preferably, a laser desorption time-of-flight mass spectrometer is used in embodiments of the invention. In laser desorption mass spectrometry, a substrate or a probe comprising markers is introduced into an inlet system. The markers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

Matrix-assisted laser desorption/ionization mass spectrometry, or MALDI-MS, is a method of mass spectrometry that involves the use of an energy absorbing molecule, frequently called a matrix, for desorbing proteins intact from a probe surface. MALDI is described, for example, in U.S. Pat. No. 5,118,937 (Hillenkamp et al.) and U.S. Pat. No. 5,045,694 (Beavis and Chait). In MALDI-MS the sample is typically mixed with a matrix material and placed on the surface of an inert probe. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in this art. The matrix dries, forming crystals that encapsulate the analyte molecules. Then the analyte molecules are detected by laser desorption/ionization mass spectrometry. MALDI-MS is useful for detecting the biomarkers of this invention if the complexity of a sample has been substantially reduced using the preparation methods described above.

Surface-enhanced laser desorption/ionization mass spectrometry, or SELDI-MS represents an improvement over MALDI for the fractionation and detection of biomolecules, such as proteins, in complex mixtures. SELDI is a method of mass spectrometry in which biomolecules, such as proteins, are captured on the surface of a protein biochip using capture reagents that are bound there. Typically, non-bound molecules are washed from the probe surface before interrogation. SELDI is described, for example, in: U.S. Pat. No. 5,719,060 (“Method and Apparatus for Desorption and Ionization of Analytes,” Hutchens and Yip, Feb. 17, 1998,) U.S. Pat. No. 6,225,047 (“Use of Retentate Chromatography to Generate Difference Maps,” Hutchens and Yip, May 1, 2001) and Weinberger et al., “Time-of-flight mass spectrometry,” in Encyclopedia of Analytical Chemistry, R. A. Meyers, ed., pp 11915-11918 John Wiley & Sons Chichesher, 2000.

Markers on the substrate surface can be desorbed and ionized using gas phase ion spectrometry. Any suitable gas phase ion spectrometers can be used as long as it allows markers on the substrate to be resolved. Preferably, gas phase ion spectrometers allow quantitation of markers.

In one embodiment, a gas phase ion spectrometer is a mass spectrometer. In a typical mass spectrometer, a substrate or a probe comprising markers on its surface is introduced into an inlet system of the mass spectrometer. The markers are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of markers or other substances will typically involve detection of signal intensity. This, in turn, can reflect the quantity and character of markers bound to the substrate. Any of the components of a mass spectrometer (e.g., a desorption source, a mass analyzer, a detector, etc.) can be combined with other suitable components described herein or others known in the art in embodiments of the invention.

In another embodiment, an immunoassay can be used to detect and analyze markers in a sample. This method comprises: (a) providing an antibody that specifically binds to a marker; (b) contacting a sample with the antibody; and (c) detecting the presence of a complex of the antibody bound to the marker in the sample.

To prepare an antibody that specifically binds to a marker, purified markers or their nucleic acid sequences can be used. Nucleic acid and amino acid sequences for markers can be obtained by further characterization of these markers. For example, each marker can be peptide mapped with a number of enzymes (e.g., trypsin, V8 protease, etc.). The molecular weights of digestion fragments from each marker can be used to search the databases, such as SwissProt database, for sequences that will match the molecular weights of digestion fragments generated by various enzymes. Using this method, the nucleic acid and amino acid sequences of other markers can be identified if these markers are known proteins in the databases.

Alternatively, the proteins can be sequenced using protein ladder sequencing. Protein ladders can be generated by, for example, fragmenting the molecules and subjecting fragments to enzymatic digestion or other methods that sequentially remove a single amino acid from the end of the fragment. Methods of preparing protein ladders are described, for example, in International Publication WO 93/24834 (Chait et al.) and U.S. Pat. No. 5,792,664 (Chait et al.). The ladder is then analyzed by mass spectrometry. The difference in the masses of the ladder fragments identify the amino acid removed from the end of the molecule.

If the markers are not known proteins in the databases, nucleic acid and amino acid sequences can be determined with knowledge of even a portion of the amino acid sequence of the marker. For example, degenerate probes can be made based on the N-terminal amino acid sequence of the marker. These probes can then be used to screen a genomic or cDNA library created from a sample from which a marker was initially detected. The positive clones can be identified, amplified, and their recombinant DNA sequences can be subcloned using techniques which are well known. See, e.g., Current Protocols for Molecular Biology (Ausubel et al., Green Publishing Assoc. and Wiley-Interscience 1989) and Molecular Cloning: A Laboratory Manual, 3rd Ed. (Sambrook et al., Cold Spring Harbor Laboratory, NY 2001).

Using the purified markers or their nucleic acid sequences, antibodies that specifically bind to a marker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). Such techniques include, but are not limited to, antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

After the antibody is provided, a marker can be detected and/or quantified using any of suitable immunological binding assays known in the art (see, e.g., U.S. Pat. Nos. 4,366,241; 4,376,110; 4,517,288; and 4,837,168). Useful assays include, for example, an enzyme immune assay (EIA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asai, ed. 1993); Basic and Clinical Immunology (Stites & Terr, eds., 7th ed. 1991); and Harlow & Lane, supra.

Generally, a sample obtained from a subject can be contacted with the antibody that specifically binds the marker. Optionally, the antibody can be fixed to a solid support to facilitate washing and subsequent isolation of the complex, prior to contacting the antibody with a sample. Examples of solid supports include glass or plastic in the form of, e.g., a microtiter plate, a stick, a bead, or a microbead. Antibodies can also be attached to a probe substrate or microchip array. The sample is preferably a biological fluid sample taken from a subject. Examples of biological fluid samples include cerebrospinal fluid, blood, serum, plasma, neuronal cells, tissues, urine, tears, saliva etc. In a preferred embodiment, the biological fluid comprises cerebrospinal fluid. The sample can be diluted with a suitable eluant before contacting the sample to the antibody.

After incubating the sample with antibodies, the mixture is washed and the antibody-marker complex formed can be detected. This can be accomplished by incubating the washed mixture with a detection reagent. This detection reagent may be, e.g., a second antibody which is labeled with a detectable label. Exemplary detectable labels include magnetic beads (e.g., DYNABEADS™), fluorescent dyes, radiolabels, enzymes (e.g., horse radish peroxide, alkaline phosphatase and others commonly used in an ELISA), and calorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker is incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

Immunoassays can be used to determine presence or absence of a marker in a sample as well as the quantity of a marker in a sample. First, a test amount of a marker in a sample can be detected using the immunoassay methods described above. If a marker is present in the sample, it will form an antibody-marker complex with an antibody that specifically binds the marker under suitable incubation conditions described above. The amount of an antibody-marker complex can be determined by comparing to a standard. A standard can be, e.g., a known compound or another protein known to be present in a sample. As noted above, the test amount of marker need not be measured in absolute units, as long as the unit of measurement can be compared to a control.

The methods for detecting these markers in a sample have many applications. For example, one or more markers can be measured to aid in the diagnosis of spinal injury, brain injury, the degree of injury, tumor due to neuronal disorders, alcohol and drug abuse, fetal injury due to alcohol and/or drug abuse by pregnant mothers, etc. In another example, the methods for detection of the markers can be used to monitor responses in a subject to treatment. In another example, the methods for detecting markers can be used to assay for and to identify compounds that modulate expression of these markers in vivo or in vitro.

Data generated by desorption and detection of markers can be analyzed using any suitable means. In one embodiment, data is analyzed with the use of a programmable digital computer. The computer program generally contains a readable medium that stores codes. Certain code can be devoted to memory that includes the location of each feature on a probe, the identity of the adsorbent at that feature and the elution conditions used to wash the adsorbent. The computer also contains code that receives as input, data on the strength of the signal at various molecular masses received from a particular addressable location on the probe. This data can indicate the number of markers detected, including the strength of the signal generated by each marker.

Data analysis can include the steps of determining signal strength (e.g., height of peaks) of a marker detected and removing “outliers” (data deviating from a predetermined statistical distribution). The observed peaks can be normalized, a process whereby the height of each peak relative to some reference is calculated. For example, a reference can be background noise generated by instrument and chemicals (e.g., energy absorbing molecule) which is set as zero in the scale. Then the signal strength detected for each marker or other biomolecules can be displayed in the form of relative intensities in the scale desired (e.g., 100). Alternatively, a standard (e.g., a CSF protein) may be admitted with the sample so that a peak from the standard can be used as a reference to calculate relative intensities of the signals observed for each marker or other markers detected.

The computer can transform the resulting data into various formats for displaying. In one format, referred to as “spectrum view or retentate map,” a standard spectral view can be displayed, wherein the view depicts the quantity of marker reaching the detector at each particular molecular weight. In another format, referred to as “peak map,” only the peak height and mass information are retained from the spectrum view, yielding a cleaner image and enabling markers with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique markers and markers which are up- or down-regulated between samples. Marker profiles (spectra) from any two samples may be compared visually. In yet another format, Spotfire Scatter Plot can be used, wherein markers that are detected are plotted as a dot in a plot, wherein one axis of the plot represents the apparent molecular mass of the markers detected and another axis represents the signal intensity of markers detected. For each sample, markers that are detected and the amount of markers present in the sample can be saved in a computer readable medium. This data can then be compared to a control (e.g., a profile or quantity of markers detected in control, e.g., normal, healthy subjects in whom tumor is undetectable).

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in pertinent part for all purposes to the same extent as if each individual publication or patent document were so individually denoted. By their citation of various references in this document, Applicants do not admit any particular reference is “prior art” to their invention.

### EXAMPLES

### Materials and Methods

### Cell Culture.

Clinical specimens of tumor biopsies were minced into small particles in DMEM/F12 medium digested with 5 mg/ml Collagenase Type II (Gibco BRL Invitrogen Corporation, Grand Island, N.Y., USA) for 3-12 hours and were passed through a 70 μm Cell Strainer (Becton Dickinson Lab Ware, Franklin Lakes, N.J. USA) to prepare single-cell suspension as was previously described (Meyers P A, Schwartz C L, Krailo M, et al. J Clin Oncol. 2005; 23(9):2004-2011). Samples were obtained with consent, using protocols approved by the institutional review boards of the University of Florida College of Medicine. (Approval #s 59-2003, 48-2004) 99-1, and 99-2 were supplied by the University of Colorado Health Sciences Center (COMIRB 98-241). The human osteosarcoma cell line MG-63 was obtained from American Type Culture Collection (ATCC, Rockville, Md.). Cells were cultured in DMEM/F12 medium supplemented with 10% (volume/volume) of characterized fetal bovine serum (FBS) (HyClone, Logan, Utah USA) at 37° C., 7.0% CO₂.

### Neurosphere/Sarcosphere System Assays.

Cells were plated at a density of 60,000 cells/well in 6-well ultra low attachment plates (Corning Inc., Corning, N.Y. USA) in 2×DMEM/F12 with methylcellulose (Sigma Chemical Co., St. Louis, Mo. USA) 1:1, final concentration 0.8%. In addition, the medium was supplemented with progesterone (20 nM), putrescine (100 μM), sodium selenite (30 nM), transferrin (25 μg/ml) insulin (20 pg/ml) (Sigma Chemical Co., St. Louis, Mo. USA) and growth factors: human-EGF (10 ng/ml), human-FGF-basic (10 ng/ml) (Pepro Tech, Rocky Hill, N.J. USA), fresh aliquots of which were added every other day. After incubation of the samples for 7-12 d, colonies containing more than 50 cells were quantitated by inverted phase contrast microscopy (Nikon Eclipse TS100) using the image program SPOT3.2.6 for MacOS.

### Semiquantitative RT-PCR.

Total RNA was isolated using the RNeasy Mini Kit (and treated with RNAase-Free DNase Set (Qiagen Sciences, Maryland, USA), according to the manufacturer's instruction and was quantitated by ultraviolet absorption using Smart Spec 3000 spectrophotometer (Bio Rad Laboratories, Hercules, Calif. USA). A SuperScript II RNase H Reverse Transcriptase first-strand synthesis system (Invitrogen Life Technologies, Carlsbad, Calif. USA) was used to synthesize the cDNA, as per manufacturers recommendations, 1.5 μg of total RNA by Oligo(dT)₁₂₋₁₈ (Invitrogen Life Technologies, Carlsbad, Calif. USA) priming. The target cDNA was amplified by using Platinum TaqDNA Polymerase (Invitrogen Life Technologies, Carlsbad, Calif. USA) and 35-37 cycles of PCR. The primers are provided in Table 1.

The PCR conditions included an initial denaturation of 1:30 min at 95° C. and subsequent denaturation for 15 sec at 95° C., annealing for 30 sec at 55 or 60° C. and extension for 55 sec at 72° C. for 35-37 cycles. Aliquots of 8 μl of the amplification products were separated by 1.2% agarose gel electrophoresis and visualized by ethidium bromide staining. Only RNA samples that gave completely negative results in PCR without reverse transcriptase were used for the analyses.

TABLE 1

| | | | Product |
|---|---|---|---|
| Gene | Forward Primer | Reverse Primer | (bp) |
| GATA4 | GCCCAAGAACCTGAATAAATCTAAG | AGACATCGCACTGACTGAGAACGTC | 208 |
| | (SEQ ID NO: 1) | SEQ ID NO: 2) | |
| GATA6 | TTCCCCCACAACACAACCTACAG | GTAGAGCCCATCTTGACCCGAATAC | 118 |
| | (SEQ ID NO: 3) | (SEQ ID NO: 4) | |
| AFP | GGTGTAGCGCTGCAAACGATG | AATTTAAACTCCCAAAGCAGCACGA | 210 |
| | (SEQ ID NO: 5) | (SEQ ID NO: 6) | |
| STAT3 | GGGTGGAGAAGGACATCAGCGGTAA | GCCGACAATACTTTCCGAATGC | 198 |
| | (SEQ ID NO: 7) | (SEQ ID NO: 8) | |
| AC133 | AATTTGTTTTGGTTTGGCATAGGAA | TGTTGTGATGGGCTTGTCATAACAG | 221 229 |
| | (SEQ ID NO: 9) | (SEQ ID NO: 10) | |
| RUNX1 | CTCAGGTTTGTCGGTCGAAGTGGAA | CCGCAGCTGCTCCAGTTCAC | 216 |
| | (SEQ ID NO: 11) | (SEQ ID NO: 12) | |
| RUNX2 | CTCCCTGAACTCTGCACCAAGTCCT | GGGGTGGTAGAGTGGATGGACG | 156 |
| | (SEQ ID NO: 13) | (SEQ ID NO: 14) | |
| RUNX3 | CCGAGCCATCAAGGTGACCGTGGAC | GGGCTGGCTGCTGAAGTGGCTTGT | 187 |
| | (SEQ ID NO: 15) | (SEQ ID NO: 16) | |
| osteo- | CCCTCACACTCCTCGCCCTATT | AAGCCGATGTGGTCAGCCAACTCGT | 259 |
| calcin | (SEQ ID NO: 17) | (SEQ ID NO: 18) | |
| ALPL | CACTGCGGACCATTCCCACGTCTT | GCGCCTGGTAGTTGTTGTGAGCATA | 206 |
| | (SEQ ID NO: 19) | (SEQ ID NO: 20) | |
| IBSP | GGGCAGTAGTGACTCATCCGAAGAA | CTCTCCATAGCCCAGTGTTGTAGCA | 166 |
| | (SEQ ID NO: 21) | (SEQ ID NO: 22) | |
| Nanog | GCTGAGATGCCTCACACGGAG | TCTGTTTCTTGACTGGGACCTTGTC | 163 |
| | (SEQ ID NO: 23) | (SEQ ID NO: 24) | |
| OCT 3A/4 | TGGAGAAGGAGAAGCTGGAGCAAAA | GGCAGATGGTCGTTTGGCTGAATA | 186 |
| | (SEQ ID NO: 25) | (SEQ ID NO: 26) | |
| NESTIN | CAGCTGGCGCACCTCAAGATG | AGGGAAGTTGGGCTCAGGACTGG | 209 |
| | (SEQ ID NO: 27) | (SEQ ID NO: 28) | |
| β-III | CTGCTCGCAGCTGGAGTGAG | CATAAATACTGCAGGAGGGC | 141 |
| TUBULIN | (SEQ ID NO: 29) | (SEQ ID NO: 30) | |
| BETA- | GCGGGAAATCGTGCGTGACATT | GATGGAGTTGAAGGTAGTTTCGTG | 229 |
| ACTIN | (SEQ ID NO: 31) | (SEQ ID NO: 32) | |

### Western Blot Analysis.

Cells were dissolved in lysis buffer containing 50 mM Tris-HCl, pH7.4, 150 mM NaCl, 1 mM EDTA, 1% NP40, 0.1% SDS, 1% Na-deoxycholate, 1 mM Na-vanadate, and protease inhibitors: 5 μg/ml pepstatin, 1 mM phenylmethylsulphonylfluoride, 10 μg/ml leupeptin, 1 mM NaF (Sigma Chemical Co., St. Louis, Mo. USA) for at least 1 hr on ice. After centrifugation (12,000 g for 10 min at 4° C.), the protein concentration of the supernatant was measured by BCA Protein Assay kit (Pierce, Rockford, Ill. USA) using Benchmark Microplate Reader (Bio Rad Laboratories, Hercules, Calif. USA). Lysates were mixed (1:1) with Laemmli Buffer (Sigma Chemical Co., St. Louis, Mo. USA). 15 μg of protein per lane were loaded into 8-16% or 10-20% Tris-HCl Ready Gels (Bio Rad Laboratories, Hercules, Calif. USA) and separated by electrophoresis and then transferred onto nitrocellulose membrane (Sigma Chemical Co., St. Louis, Mo. USA). Membranes were blocked 1 hr at room temperature (RT) and incubated over night at 4° C. with the corresponding antibodies in 5% bovine albumin, (Sigma Chemical Co., St. Louis, Mo. USA), Tris-Buffered Saline (TBS) and 0.1% Tween-20 (Bio-Rad Laboratories, Hercules, Calif. USA). After being washed 6 times in TBS with 0.1% Tween20, blots were incubated with peroxidase-conjugated goat antibodies to mouse or rabbit IgG (Cell Signaling Technology) or rabbit antibodies to goat IgG (Jackson Immuno Research Laboratories, West Grove, Pa. USA). Immunoreactive bands were detected by ECL Plus Western Blotting Detection Reagents (Amersham Biosciences UK) for 60 sec. Primary antibodies:—Anti-STAT3 (R&D Systems), Phospho-Stat3(Tyr705) (Cell Signaling Technology), β̅III Tubulin (BAbCO, Berkeley, Calif. USA), Anti-α-Actin (Sigma Chemical Co., St. Louis, Mo. USA), AFP (C-19) and Oct-3/4 (N-19) (Santa Cruz Biotechnology).

### Immunofluorescent Staining:

Cells were grown 3-5 days on glass cover slips coated with polyornithine (10 μg/ml)/laminin (5 μhg/ml) in DMEM/F12 medium with 10% FBS in 12-well plates. Cells were fixed in freshly prepared cold 4% paraformaldehyde (Sigma Chemical Co., St. Louis, Mo. USA) for 15 min at RT and permeabilized with ice cold 0.5% Triton-X100, 2% sucrose in DPBS for 5 min. After blocking 20 min with 25% goat serum (Sigma Chemical Co., St. Louis, Mo. USA) in DPBS cells were incubated for 30 min at RT in 25% goat serum in DPBS with primary antibodies: Nanog (Mitsui et al., Cell, 113(5):631-42, 2003), Tubulin (BAbCO, Berkeley, Calif. USA), Stro-1 (Developmental Studies Hybridoma Bank, Department of Biological Sciences, the University of Iowa, Iowa City, Iowa USA) and visualized by an indirect immunofluorescence microscopy with secondary anti-mouse. All proteins were visualized by indirect immunofluorescence using Alexa red-conjugated antibody (Molecular Probes, Eugene, Oreg.). For F-actin visualization, cells were incubated with 0.1 μg/ml of TRITC-labeled phalloidin (Jackson Lab)

### Histological Analyses and Immunohistochemistry.

Formalin fixed paraffin embedded tissue sections (5 μm) were sequentially deparaffinized, rehydrated and blocked for endogenous peroxidase activity. Following a 95° C., 25 minute antigen retrieval in Trilogy unmasking solution (Cell Marque, Hot Springs Ark.). Slides were biotin blocked, serum blocked and immunostained using a goat ABC Elite Kit (Vector Labs, Burlingame, Calif.). Antibodies to Oct 3/4 and Nanog (R&D Systems, Minneapolis, Minn.) were applied at 1:50 for one hour at room temperature. Positive staining was detected with DAB (3,3′-Diaminobenzidene) and light green SF yellowish (Sigma, St. Louis, Mo.) was used as the counterstain.

### Differentiation Staining

Cells derived from bone sarcoma spheres were grown on adhesive substrate in adipogenic and osteogenic media for 14 days. Lipid induced cells were fixed with 10% formalin, washed and then stained with Oil red 0 for 7 minutes. Hematoxylin was used as the counterstain. Cells in osteogenic medium were fixed in ethanol followed by staining with Silver nitrate solution and exposed to sunlight for 20 minutes. Cells were washed three times in distilled water and then counterstained with Nuclear Fast Red.

### Statistical Analyses.

Average of densitometry measurements were compared using student's t-test. Significance of Oct 3/4 and Nanog co-expression correlation determined by calculation of correlation coefficient (Statistica, StatSoft, Tulsa, Okla.).

### Example 1

### Stem Cells in Bone Sarcomas

To determine whether bone sarcomas might contain stem-like cells, cultures from biopsies of untreated chondrosarcoma and osteosarcoma were first established, as well as the established cell line MG-63, and evaluated each for their ability to generate spherical clones (sarcospheres) and self renew in the neurosphere culture system. All nine initial bone sarcoma cultures and the MG-63 cell line formed spherical colonies that were termed “sarcospheres” at an epigenetic frequency of 10⁻²-10⁻³ when plated at a clonigenic density of 60,000 cells per well in six well plates (See FIG. 1). This frequency is similar to that reported by others for brain and breast malignancies (Ignatova T N, et. al. Glia 2002; 39(3):193-206; Hemmati H D, et al. Proc Natl. Acad. Sci. USA 2003; 100(25):15178-83.). Sarcospheres were also generated at a similar frequency from fresh tumor dissociates generated at the time of biopsy. This frequency suggests an epigenetic rather than a mutational mechanism underlying the ability of these cells to form clonal colonies under our culture conditions.

### Example 2

### Self-Renewal of Cells in Sarcospheres

We then asked whether these clone-forming cells could self-renew in this sarcosphere system. Cultured sarcospheres were dissociated to single cells from four representative cultures (OS 521, OS 01-187, OS-99-1 and CS 828) and allowed to grow as a monolayer adherent culture. At near confluence the cells were harvested and reseeded into suspension culture. All four cultures examined were able to demonstrate self-renewal by the formation of secondary spheres at a similar or increased frequency of approximately 10⁻². One representative culture, OS-521, was serially re-cloned for four passages and continued to generate spheres at a frequency of 10⁻². These data show that bone sarcomas share, with ectodermal tumors, the ability to generate suspended clones under growth constraining conditions, suggesting the presence of a small subpopulation of self-renewing, pluripotent cells.

In the embryonic setting, self-renewal is associated with pluripotency, or the ability to maintain the expression of genes specific for multiple cellular lineages, and then differentiate along one of those lineages while restricting the others. Self-renewal and pluripotency of undifferentiated ES cells may be maintained by a crosstalk involving three transcription factors: the POU family member Oct 3/4, the recently identified homeoprotein Nanog, and activated Stat 3. To further confirm the stem cell-like nature of these cells, experiments were conducted to determine whether these pathways might be active in bone sarcoma cultures grown without exogenous LIF.

Evaluation of mRNA expression levels revealed expression of all three transcription factors. Both spheres and adherent cultures demonstrated similar expression levels of Stat 3. The relative expression of Oct 3/4, Nanog, in cells grown as adherent culture supplemented with serum versus sarcospheres grown in the serum starved anchorage independent system, was determined using semi-quantitative RT-PCR with β-actin as a control. Strikingly, substrate attached cultures showed significantly (p<0.05) lower expression of Oct 3/4, and Nanog. This is similar to the Oct 3/4 and Nanog down regulation seen under differentiation permissive conditions in embryonic tissue settings. Oct 3/4 protein expression in adherent cultures is further demonstrated by Western blot analysis. Expression of Stat-3 protein and its activated form (phosphorylated at Tyr 705) was detected both in sarcospheres and adherent serum supplemented LIF-free cultures using both RT-PCR and Western blot analyses. These data demonstrate, for the first time, that bone sarcoma cultures have constitutively activated Stat 3 similar to that of other malignancies. Unambiguous nuclear localization of the transcription factor Nanog was observed in almost all cells examined by immunocytochemistry.

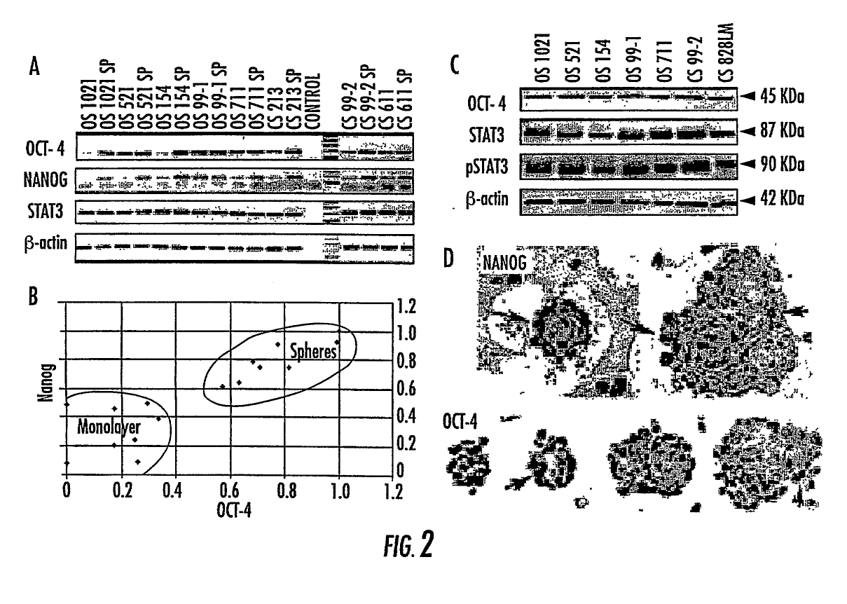

FIGS. 2A-2D show that genes specific to ESCs show increased expression in sphere cultures derived from bone sarcomas. FIG. 2A is a Western blot showing monolayer and sarco-sphere (SP) cultures from five osteosarcoma (OS) and three chondro-sarcomas (CS) were analyzed for Oct-4, Nanog and STAT3 mRNA by RT-PCR; β-actin expression was used as a positive control. Sphere cultures demonstrate increased transcription of both Oct-4 and Nanog over adherent cultures; STAT3 expression was uniform between both culture types. FIG. 2B is a densitometer scan showing relative band intensities for Oct-4 and Nanog for each culture from FIG. 2A were quantitated by densitometry, normalized relative to β-actin and plotted on the graph shown (Oct-4, x-axis; Nanog, y-axis). As indicated by the grouping, the sphere cultures of each sarcoma showed significantly greater expression of both Oct-4 and Nanog than adherent monolayer cultures (p<0.05, Pearson's correlation). FIG. 2C is a Western blot analysis of lysates from representative bone sarcoma cell cultures for protein expression of Oct-4, STAT3 and activated (phosphorylated, p) STAT3. β-actin was used as a positive control for loading, membrane transfer and immunoblotting. All cultures showed positive staining of protein bands of the appropriate sizes as indicated. FIG. 2D is a scan of a photograph showing small and large sarcospheres embedded in fibrin and then paraffin and stained using immunohistochemistry for Nanog and Oct-4 as indicated. Small spheres show intense staining in cells in the periphery. Large spheres show similar numbers of darkly staining cells with dramatically increased numbers of poorly staining cells in the interior of the sphere.

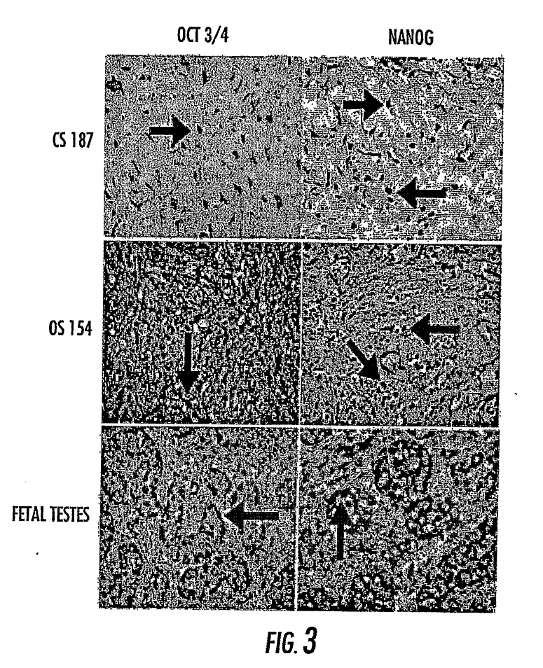

Next, it was addressed whether Oct and Nanog are expressed in actual tumor tissue. Paraffin tissue block sections were evaluated from eight patients using immunohistochemical analyses. Nanog and Oct 3/4 nuclear staining was demonstrated in seven of the eight tumors studied (FIG. 3). The stained nuclei were of malignant cells, and not from infiltrating normal cells by histologic criteria. The numbers of Oct and Nanog positive cells varied between 1-25% and 1-50% respectively. The nuclear staining for Oct and Nanog in these tissue specimens implies a role for these transcription factors in-vivo rather than an artifact of in-vitro culture conditions.

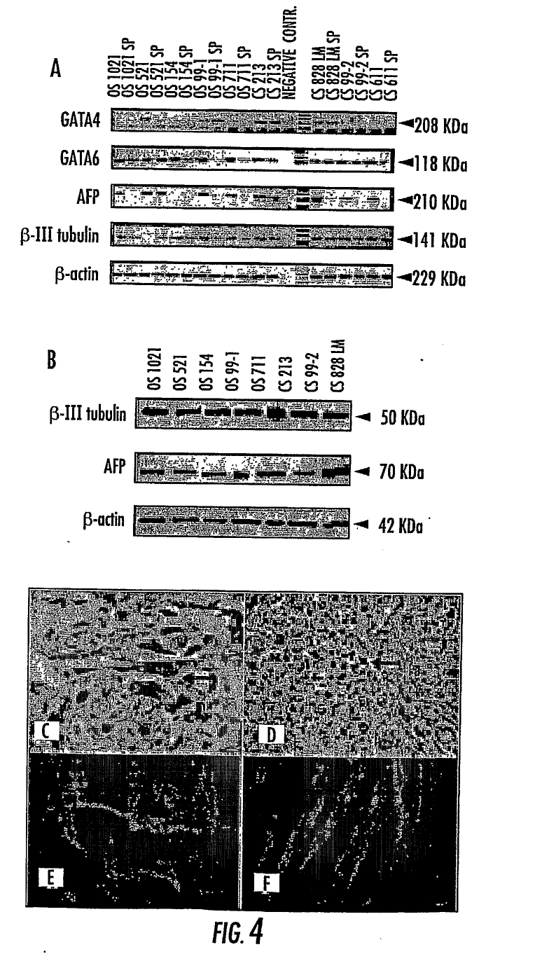

Based on these findings, it was reasoned that if bone sarcomas utilize some of the molecular machinery of ES cells, including activated Stat-3, Oct3/4 and Nanog, it stands to reason that they might express genes normally associated with the other cellular lineages, i.e. endoderm and ectoderm as opposed to mesoderm. This is depicted in FIG. 4A where RT-PCR reveals expression of alpha fetoprotein (AFP), gata-4 and gata-6, genes indicative of endodermal differentiation, as well as β-III tubulin which is thought to be a marker of neural-ectoderm, but has also been demonstrated in some poorly differentiated malignancies. Alpha feto-protein and p-III tubulin production was also demonstrated by Western analysis (FIG. 4B). P-III tubulin was found both in tumor paraffin sections (FIGS. 4 C and D) and culture (FIGS. 4 E and F).

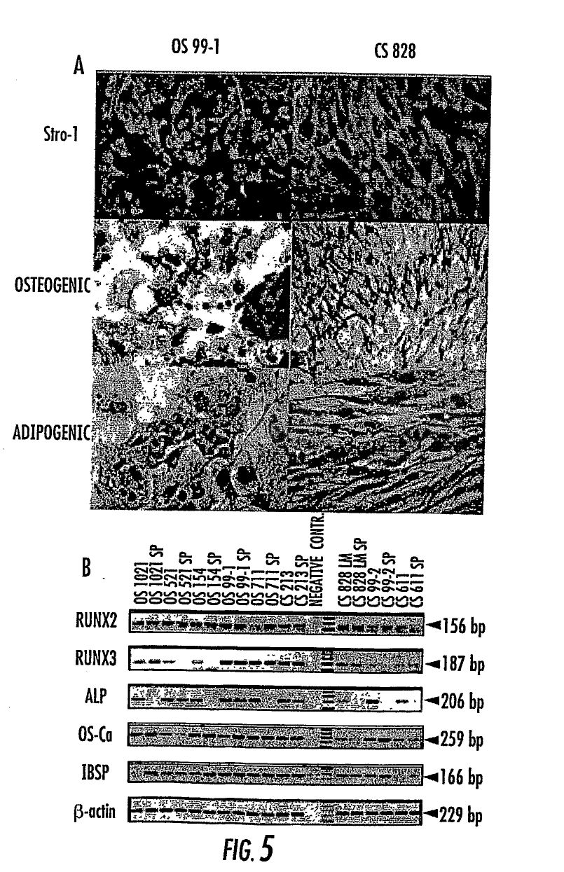

Although the bone sarcomas studied here express transcription factors associated with embryonic stem cells, their histologic phenotype is one of arrested mesenchymal differentiation. It was next determined whether Stro-1 could be detected. Stro-1 is a cell surface protein associated with bone marrow stromal cells, also known as mesenchymal stem cells. Mesenchymal stem cells have been shown to differentiate along various mesenchymal lineages such as bone, cartilage, fat and muscle. Stro-1 has been shown to be expressed in the permanent osteosarcoma cell line MG-63. Immunocytochemistry revealed that 2-10% of adherent cells in both chondrosarcoma and osteosarcoma cultures were positive for the Stro-1 surface protein (FIG. 5 A). There were not any obvious morphologic differences between the cells immunopositive or negative for Stro-1. Analysis of substrate-attached cultures grown in media supplemented with serum and differentiation factors revealed features of differentiation along two mesenchymal lineages—adipogenic and osteogenic (FIG. 5A). These findings correlate well with the RT-PCR results demonstrating expression of multiple mesenchymal lineage markers, e.g. bone sialo-protein, osteocalcin, alkaline phosphotase, and runx 2 and 3. (FIG. 5B).

The results presented here indicate that there exists in bone sarcomas a subpopulation of stem-like cells. These cells have the capacity to self-renew and express the key determinants of ES cell pluripotency: Oct 3/4, Nanog, and activated Stat-3 suggesting the successful appropriation of molecular machinery characteristic of ES cells. In addition these cells express genes associated with mesoderm, ectoderm and endoderm. They demonstrate the mesenchymal stem cell surface marker Stro-1 and can be induced to differentiate along at least two mesenchymal lineages.

Without wishing to be bound by theory, these results support the hypothesis that a small number of cells within the bulk of a solid tumor are responsible for the initiation and growth of the malignancy, and suggests a possible mechanism for this stem cell-like behavior. Consequently, cytotoxic chemotherapeutic agents, developed in part by assessing partial response of the gross tumor mass, may not address this small proportion of tumorigenic cells. This could explain the relative resistance of osteosarcoma to chemotherapy in which survival with chemotherapy alone is only 20%, and the near complete resistance of chondrosarcoma to standard drug therapy.

The novel findings presented here suggest the transcription factors Oct 3/4, Nanog, Stat-3 may be part of the mechanism by which these cells maintain “stemness”. Exploring the role of Stro-1 and other surface markers associated with mesenchymal precursors may allow us to prospectively identify the tumorigenic cells in bone sarcomas. Perhaps more importantly, these three transcription factors and Stro-1 could all serve as potential targets for selective non-cytotoxic therapy in bone sarcoma patients since these tumors can be resistant to current therapeutic protocols.

In summary, using growth conditions favorable only to clonogenic stem/progenitor cell populations, yielded in the present study the presence of a clonogenic stem-like cell from human bone sarcomas that has many attributes in common with both normal adult human stem cells and glioblastoma stem cells.

### Example 3

### Tumors from Sarcospheres

Tumors from sarcospheres were established from a human sarcoma biopsy and from a canine osteosarcoma cell line. See, FIGS. 6A-6B, which demonstrate SCID mice bearing tumors after subcutaneous injection of sarcospheres. Thus, the tumorigenic cell is preserved within the sphere culture system.

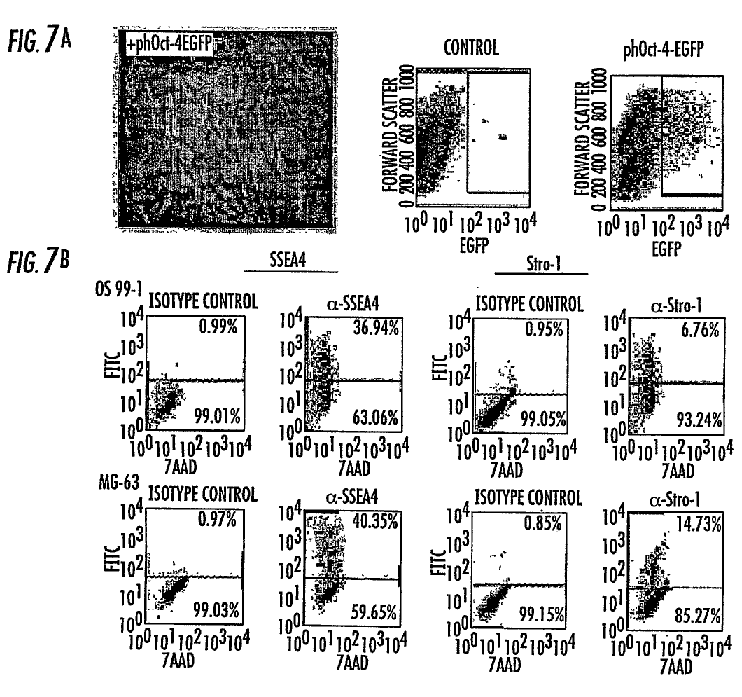

We have demonstrated the existence of a small sub-population of cells within sarcoma adherent culture that express the embryonic stem cell transcription factor Oct 3/4, also known as Oct 4. We have confirmed via flow cytometry that there exists within adherent sarcoma cultures a subpopulation of cells that express SSEA-4, Oct 4, and Stro-1, markers of embryonic and adult stem cells. This is represented in the figure below for Oct 4, SSEA 4 and Stor-1 as FIG. 7B.

The plasmid construct, phOct4-EGFP, obtained from Dr. Wei Cui of the Rosalin Institute, UK, containing the human Oct-4 promoter sequence (spanning −3917 to +55, relative to the transcription start site) linked to the coding sequence for enhanced green fluorescent protein (EGFP). Human ESCs are stably transfected with this construct, EGFP expression driven by the Oct-4 promoter faithfully represents expression of Oct-4 in undifferentiated ESCs and during their differentiation. EGFP expression co-localized with endogenous Oct-4 protein as well as surface antigens SSEA-4 and Tra-1-60. Neural differentiation of the cells as well as targeted knockdown of endogenous Oct-4 expression by RNAi, down regulated EGFP and correlated closely with the reduction in endogenous Oct-4 protein. This was confirmed using RT-PCR and immunohistochemistry that a subpopulation of cells in cultures from bone sarcomas expresses Oct-4. In accordance with these results, we found that following transient transfection of the sarcoma cultures with phOct4-EGFP, a range of about 2-5% of the cells among the different cultures were EGFP⁺ by fluorescence microscopy and flow cytometry (FIGS. 7A and B). Parallel transfections using a plasmid construct containing EGFP driven by the cytomegalovirus (CMV) promoter/enhancer indicated a transfection efficiency of about 15-25% Resulting expression of CMV-driven GFP is stable in both monolayer and sphere cultures

Experimental protocol for determining tumorigenicity of cells from spheres vs cells from monolayer in SCID mice: For each tumor sample, cells are isolated from ˜1 cubic cm of tumor from an open biopsy taken from patients that have not previously received any form of chemotherapy. Frozen section analysis of an adjacent area of the biopsy specimen performed for diagnostic purposes will confirm the histologic diagnosis prior to cell culture preparation. From the biopsy, the tissue matrix is enzymatically digested, and the cells isolated. The cells from the tumor (or existing cell line/cultures) are seeded into several 25 cm flasks. At ˜65% confluence one of the flasks is infected with concentrated, VSV-G pseudotyped LV.GFP-neo; this typically results in transduction of >70% of the cells. (The cells in the remaining flasks will be frozen and stored in liquid nitrogen for further experiments) At confluence the lentivirally infected cultures are trypsinized, and split into several flasks in medium supplemented with G418 to enable positive selection of the transduced cells. This provides a pure population of GFP+ cells. At confluence, the GFP+ cells are divided and seeded into separate monolayer and sarcosphere cultures. After 14-21 days, the resulting spheres are collected, disaggregated, and the cells counted. In parallel, the cells from the monolayers are trypsinized washed and counted. Afterward, the cells are resuspended in a minimal volume of saline solution, and 3×10⁵ cells are injected subcutaneously into the backs of two groups of 10 NOD/SCID mice.

Following injection, the animals are monitored daily for 12 weeks. The skin of the back is palpated for tumor growth, and the diameter of the resulting tumors are measured using specialized calipers and recorded. For each tumor type or cell line, the numbers of animals with tumors, days to tumor onset, and tumor size are scored and compared between the groups receiving monolayer or sarcosphere cells. These values are used to establish the relative tumor forming capacity of the sarcospheres. (During the experiment, the animals are maintained according to University of Florida IACUC guidelines for tumor studies in experimental animals.)

From our preliminary experiments, 3×10⁵ cells is our current working dose; however, our initial experiments involve the MG-63 and OS 99-1 cells to enable further optimization of conditions for the xenograft model. For different tumors the cell dose may need to be adjusted upward or downward as results dictate to enable discrimination of the relative tumor forming capacity of the sarcosphere cells vs. monolayer cells.

OCT 4, Stro-1 and other stem marker enrichment: We have found that a subpopulation of cells in osteosarcomas express the ESC-restricted homeodomain protein Oct-4. Oct-4 expression is used to enable fractionation of ESC-like cells from osteosarcoma cell cultures and then test the relative tumor forming capacity of the respective cell fractions: Oct-4 enriched vs. Oct-4 depleted. We have recently obtained a plasmid construct whereby the human Oct-4 promoter sequence drives expression of EGFP. Delivery of this construct to osteosarcoma cells enables the identification of living cells that express Oct-4 by their green fluorescence, which is easily distinguished by microscopy and FACS.

For these experiments, a portion of the cells are thawed from each of the cultures of our osteosarcoma cells and transduce each culture with a lentiviral vector containing the hOct-4-EGFP construct (LV.hOct-4-EGFP-neo). In addition to the Oct-4/EGFP reporter, the vector also contains a second downstream cistron containing the neomycin phosphotransferase coding region (neo) driven by the SV40 promoter. Expression of the neo gene by a cell confers resistance to neomycin and its analog G418. Typically, our pseudotyped, concentrated, lentiviral vector preparations provide a transduction efficiency of osteosarcoma cells of >70%. While the vector will transduce the majority of the osteosarcoma cells, only those cells that express Oct-4 will also express EGFP. Therefore, to eliminate any contaminating Oct-4+ cells that were not transduced by the lentivirus and thus do not fluoresce, the infected cultures are placed under positive selection in medium with G418. The selected cultures are then be expanded, and a portion of each is analyzed periodically by fluorescence microscopy and flow cytometry to determine the percentage of GFP+ cells. This is done for each source of tumor cells to ensure that sufficient numbers of Oct-4+ cells are present prior to initiating animal experimentation.

Immediately prior to injection, the cells in the cultures are trypsinized, washed and fractionated by FACS, enabling separation of the EGFP+(Oct-4+) cells from those that are GFP-(Oct-4−). The two groups of cells will then be xenografted into separate groups of 10 NOD/SCID mice, (3×10⁵ cells per mouse) and the relative tumor forming capacity of the Oct-4+ and Oct-4 depleted cells in each tumor cell culture are determined. For each tumor type or cell line, the numbers of animals with tumors, days to tumor onset, and tumor size are scored and compared between the groups receiving Oct 4+ tumor cells and Oct-4 depleted cells. At the time of sacrifice, either because of tumor burden or at the conclusion of the 12-week experiment, the resulting tumors or tissues near the site of injection are harvested, along with the lungs. The tissues are fixed in 4% paraformaldehyde, paraffin embedded and sectioned. Within the sections we examine any tumors for their morphology and the appearance of osteoid. We also analyze the tumors with respect to the GFP/Oct-4 positive cells for their relative number and locations within the tumors. Sections from all tissue samples are stained for Oct-4 expression using immunohistochemistry.

The ESC-like cells in osteosarcoma transduced with the lentiviral delivery of the Oct-4-EGFP construct has the potential to be an extremely powerful tool. It has the potential to give unique insight into the formation of tumors, the differentiation of cancer stem cells and the relative locations and resident number of osteosarcoma stem cells within a forming tumor. It will also enable the ready identification of osteosarcoma stem cells for subsequent passage and experimentation.

We have observed multipotent cells in our existing bone sarcoma cultures and have previously screened them for surface antigens found on MSCs. We found that >75% of bone sarcoma cells are positive for CD 90, CD 29, CD 73 and CD 44, while as many as half are CD 105⁺. Individually, these antigens are found on a variety of cell types and lineages, and their high frequency here is unlikely to enable significant enrichment of a cancer stem cell by antigen-specific cell fractionation. The anti-Stro-1 antibody however was raised specifically against human bone marrow stromal cells and is frequently used to identify and isolate multipotent mesenchyrnal precursors. We found the Stro-1 antigen on between 5-15% of the cells in our respective bone sarcoma cells We will enrich for cells bearing the Stro-1 antigen from osteosarcomas and evaluate their relative tumor forming potential. Our approach will be similar to that described above for ESC-like cells. In these experiments, cells bearing the Stro-1 surface marker will be fractionated from each of the cultures of osteosarcoma cells. For each osteosarcoma culture the relative tumor forming potential of the two cell fractions, Stro-1 enriched vs. Stro-1 depleted, will be compared in the NOD/SCID mouse model.

Cells from osteosarcoma are plated in monolayer. For each culture an initial screen from a portion of the cells is performed to determine the approximate percentage that are Stro-1 positive. This is recorded, and used to determine the total number of cells needed to attain the necessary dosage to deliver to the NOD/SCID mice. At the time of injection, the cultures are trypsinized, and incubated with the Stro-1 antibody and then an appropriately labeled secondary antibody. The cells are sorted into Stro-1+ and Stro-1− fractions, and 3×10⁵ cells of each is delivered to separate groups of 10 NOD/SCID mice. Tumor formation over a 12 week period is evaluated as described. At the time of sacrifice, any resulting tumors, as well as tissues from the injection site of all animals without tumors are collected as are the lungs. The tissues are fixed, embedded in paraffin and sectioned. Tissue sections are stained for Stro-1 as well as hematoxylin and eosin. These experiments will be repeated for SSEA-4 and other candidate markers such as CXCR4.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims

What is claimed is:

1. An isolated or purified population of pluripotent mammalian stem cells comprising:
at least one embryonic stem cell marker on the surface of the stem cells, wherein the embryonic stem cell marker is Oct 3/4 or Nanog; and,
at least one stem cell marker comprising, activated Stat-3 CXCR4, CD 133, SCA-1, Tra-1-60, CD 44, CD 73, CD 90, CD 105 and Stro-1; wherein,
the purified population of mammalian pluripotent stem cells, is substantially free of cells that do not express the stem cell markers.

2. The isolated or purified population of mammalian pluripotent stem cells of claim 1, wherein the stem cell markers comprise Oct 3/4, Nanog, and activated Stat-3.

3. The isolated or purified population of mammalian pluripotent stem cells of claim 1, wherein the stem cells are isolated from sarcomas.

4. The isolated or purified population of mammalian pluripotent stem cells of claim 3, wherein the sarcoma is osteosarcoma or chondrosarcoma.

5. The isolated or purified population of mammalian pluripotent stem cells of claim 1, wherein the stem cells are Oct 3/4⁺.

6. The isolated or purified population of mammalian pluripotent stem cells of claim 1, wherein the stem cells are activated Stat-3⁺.

7. The isolated or purified population of mammalian pluripotent stem cells of claim 1, wherein the stem cells are Nanog⁺.

8. The isolated or purified population of mammalian pluripotent stem cells of claim 1, wherein the stem cells are Lin⁻.

9. A method of isolating stem cells from sarcomas comprising:
isolating a sarcoma;
dissociating cells from the sarcoma;
culturing the cells on a non-adhesive substrate in anchorage independent serum free media;
adding hormones and growth factors;
adding methyl cellulose; thereby,
isolating stem cells.

10. The method of claim 9, wherein the hormone is progesterone.

11. The method of claim 9, wherein the growth factors comprise epidermal growth factor (EGF) and fibroblast growth factor.

12. The method of claim 9, wherein the sarcoma is isolated from a mammal.

13. The method of claim 9, wherein the sarcoma is chondrosarcoma or osteosarcoma.

14. The method of claim 9, wherein the stem cells are identified by markers comprising: Oct 3/4, Nanog, activated Stat-3, CXCR4, CD 133, SCA-1, Tra-1-60, CD 44, CD 73, CD 90, CD 105 and Stro-1.

15. A method of treating tumors comprising:
targeting stem cells expressing at least one marker selected from: Oct 3/4, Nanog, activated Stat-3, CXCR4, CD 133, SCA-1, Tra-1-60, CD 44, CD 73, CD 90, CD 105 and Stro-1;
inhibiting proliferation and/or lysing the stem cells; and,
inhibiting stem cell migration to a tumor and/or stem cell differentiation in a tumor; wherein,
growth and/or metastasis of a tumor is inhibited.

16. The method of claim 15, wherein the stem cells are targeted with antibodies directed to any one marker selected from: Oct 3/4, Nanog, activated Stat-3, CXCR4, CD 133, SCA-1, Tra-1-60, CD 44, CD 73, CD 90, CD 105 and Stro-1.

17. The method of claim 15, wherein the antibodies are fused to therapeutic effector molecules.

18. The method of claim 17, wherein the therapeutic effector domain is selected from the group consisting of endostatin, angioarrestin, angiostatin (plasminogen fragment), anti-angiogenic antithrombin III, cartilage-derived inhibitor (CDI), CD59 complement fragment, fibronectin fragment, gro-beta, heparinases, heparin hexasaccharide fragment, human chorionic gonadotropin (hCG), interferon alpha/beta/gamma, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-β), vasculostatin, and vasostatin (calreticulin fragment).

19. The method of claim 17, wherein the therapeutic effector domain is endostatin, angiostatin, basement-membrane collagen-derived anti-angiogenic factors tumstatin, canstatin, or arrestin.

20. The method of claim 17, wherein the therapeutic effector domain comprises chemokines, radionuclides and/or interferon.

21. The method of claim 17, wherein the nuclides are ⁹⁰Y, ¹³¹I, ¹¹¹In, ¹²⁵I.

22. The method of claim 17, wherein the therapeutic effector domain is a cytolytic molecule.

23. The method of claim 22, wherein the cytolytic molecule is TNF, enzymes, mediators of apoptosis and/or toxin.

24. The method of claim 23, wherein the toxin is selected from the group consisting of as ricin, abrin, diphtheria, gelonin, Pseudomonas exotoxin A, Crotalus durissus terrificus toxin, Crotalus adamnenteus toxin, Naja naja toxin, and Naja mocambique toxin.

25. The method of claim 23, wherein the mediators of apoptosis include ICE-family of cysteine proteases, apoptin, Bcl-2 family of proteins, Bax, bclXs and caspases.

26. A method of treating a tumor comprising:
isolating stem cells from a patient, whereby the stem cells are identified by Oct 3/4, Nanog, activated Stat-3, CXCR4, CD 133, SCA-1, Tra-1-60, CD 44, CD 73, CD 90, CD 105 and Stro-1; and,
isolating antigen specific lymphocytes from the patient; and,
co-culturing the stem cells and lymphocytes ex-vivo thereby priming the lymphocytes; and,
isolating activated lymphocytes from the co-culture; and,
re-infusing the activated lymphocytes into the patient; thereby,
activating an immune response to said stem cells and preventing self-renewal of the tumor.

27. The method of claim 26, wherein the antigen-specific lymphocytes are T- or B-lymphocytes.

28. The method of claim 26, wherein the antigen-specific lymphocytes are CD4⁺ and/or CD8⁺.

29. A method of treating a tumor comprising:
isolating stem cells from a patient, whereby the stem cells are identified by Oct 3/4, Nanog, activated Stat-3, CXCR4, CD 133, SCA-1, Tra-1-60, CD 44, CD 73, CD 90, CD 105 and Stro-1; and,
isolating antigen presenting cells from the patient; and,
co-culturing the stem cells and antigen presenting cells ex-vivo; and,
isolating the antigen presenting cells from the co-culture; and,
re-infusing the antigen presenting cells into the patient; thereby,
activating an immune response to said stem cells and preventing self-renewal of the tumor.

30. The method of claim 29, wherein the antigen presenting cells are dendritic cells.

31. The method of claim 29, wherein the antigen presenting cell is an immature dendritic cell.

32. A method of treating cancer comprising:
administering at least one protein or peptide to a patient comprising Oct 3/4, Nanog, activated Stat-3, CXCR4, CD 133, SCA-1, Tra-1-60, CD 44, CD 73, CD 90, CD 105 and Stro-1; thereby stimulating an immune response against stem cells expressing said proteins.

33. A method of selectively treating cancer comprising:
administering to a patient in need of treatment a therapeutically effective amount of a composition comprising antigen-activated dendritic cells, wherein dendritic cells are produced from proliferating cell cultures by a method comprising providing a tissue source comprising dendritic cell precursors;
culturing the tissue source on a substrate and in culture medium to expand the number of dendritic cell precursors by allowing the dendritic cell precursors to proliferate; wherein,
said culture medium comprises GM-CSF and at least one other factor which inhibits the proliferation or maturation of non-dendritic cell precursors thereby increasing the proportion of dendritic cell precursors in the culture; and,
culturing the dendritic cell precursors for a period of time sufficient to allow them to mature into mature dendritic cells;
wherein the dendritic cells are pulsed with stem cell derived antigen; wherein,
the dendritic cells process the stem-cell derived antigen to produce modified self-protein antigen, which is expressed by the dendritic cells and which activate an in vivo immune response which destroys stem cells expressing the stem-cell derived antigen.

34. The method of claim 33, wherein the stem-cell derived antigen comprises Oct 3/4, Nanog, activated Stat-3, CXCR4, CD 133, SCA-1, Tra-1-60, CD 44, CD 73, CD 90, CD 105 and Stro-1.

35. The method of claim 33, wherein immune cells selectively target stem cells expressing Oct 3/4, Nanog, activated Stat-3, CXCR4, CD 133, SCA-1, Tra-1-60, CD 44, CD 73, CD 90, CD 105 and Stro-1.

36. The method of claim 35, wherein the immune cells are CD4⁺ T lymphocytes.

37. The method of claim 35, wherein the immune cells are CD8⁺ T lymphocytes.

38. A method of identifying candidate therapeutic compounds comprising:
culturing stem cells expressing at least one marker selected from: Oct 3/4, Nanog, activated Stat-3, CXCR4, CD 133, SCA-1, Tra-1-60, CD 44, CD 73, CD 90, CD 105 and Stro-1 with a candidate therapeutic agent;
identifying candidate therapeutic agents which inhibit proliferation or growth, and/or lyse the stem cells and/or inhibit stem cell migration to a tumor and/or stem cell differentiation in a tumor; wherein,
growth and/or metastasis of a tumor is inhibited, and identifying a candidate therapeutic agent.

39. The method of claim 39, wherein a candidate therapeutic agent comprises organic molecules, inorganic molecules, vaccines, antibodies, nucleic acid molecules, proteins, peptides and vectors expressing nucleic acid molecules.

40. A method of treating cancer comprising:
administering antibodies to stem cell biomarkers wherein the antibodies are specific for at least one marker selected from: Oct 3/4, Nanog, activated Stat-3, CXCR4, CD 133, SCA-1, Tra-1-60, CD 44, CD 73, CD 90, CD 105 and Stro-1 and/or ligands thereof;
inhibiting proliferation or growth, and/or lysing the stem cells and/or inhibiting stem cell migration to a tumor and/or stem cell differentiation in a tumor; wherein, growth and/or metastasis of a tumor is inhibited, and treating cancer.

41. A method of producing self-renewing pluripotent stem cell clones comprising:
culturing sarcospheres;
dissociating sarcospheres into single cells;
culturing said single cells to near confluence and harvesting;
reseeding harvested cells into suspension cultures;
identifying self-renewing cells by formation of secondary spheres;
thereby producing self-renewing pluripotent stem cell clones.

42. The method of claim 41, wherein the self-renewing pluripotent stem cell clones express at least one of: Oct 3/4, Nanog, activated Stat-3, CXCR4, CD 133, SCA-1, Tra-1-60, CD 44, CD 73, CD 90, CD 105 and Stro-1.
